(19) United States
(12) Patent Application Publication
(10) Pub. No.: US 2018/0207119 A1
(43) Pub. Date: Jul. 26, 2018

(54) AMINO ACID COMPOSITIONS AND METHODS FOR THE TREATMENT OF MUSCLE DISEASES AND DISORDERS

(71) Applicant: AXCELLA HEALTH INC., Cambridge, MA (US)

(72) Inventors: Michael Hamill, Wellesley, MA (US); Raffi Afeyan, Boston, MA (US); William Comb, Malden, MA (US)

(21) Appl. No.: 15/847,343

(22) Filed: Dec. 19, 2017

Related U.S. Application Data
(60) Provisional application No. 62/576,321, filed on Oct. 24, 2017.
(60) Provisional application No. 62/545,358, filed on Aug. 14, 2017.
(60) Provisional application No. 62/491,776, filed on Apr. 28, 2017.
(60) Provisional application No. 62/443,205, filed on Jan. 6, 2017.
(60) Provisional application No. 62/436,073, filed on Dec. 19, 2016.

(51) Int. Cl. A61K 31/198; A61P 21/00; A61K 38/05; A61K 38/06; A61K 31/4172

(52) U.S. Cl. CPC A61K 31/198; A61P 21/00; A61K 31/4172; A61K 38/06; A61K 38/05

(57) ABSTRACT

This disclosure provides compositions comprising amino acid entities. The disclosure also provides methods for enhancing muscle function comprising administering an effective amount of the compositions to a subject in need thereof.

### RELATED APPLICATIONS

This application claims priority to U.S. Ser. No. 62/436,073 filed Dec. 19, 2016, U.S. Ser. No. 62/443,205 filed Jan. 6, 2017, U.S. Ser. No. 62/491,776 filed Apr. 28, 2017, U.S. Ser. No. 62/545,358 filed Aug. 14, 2017, and U.S. Ser. No. 62/576,321 filed Oct. 24, 2017, the contents of which are each incorporated herein by reference in their entireties.

### BACKGROUND

There are a number of diseases and disorders associated with the decrease or degenerative loss of muscle mass. Muscle atrophy is associated with a number of serious diseases, such as cancer, AIDS, renal failure, liver disease, and congestive heart failure. Furthermore, disuse of muscles through immobilization or aging also results in muscle atrophy.

Sarcopenia is a disease characterized by degenerative loss of skeletal muscle mass (typically 0.5-1% loss per year after the age of 25), quality, and strength associated with aging. Sarcopenia is a component of the frailty syndrome. Frailty is a common geriatric syndrome that embodies an elevated risk of catastrophic declines in health and function among older adults. Contributors to frailty can include sarcopenia, osteoporosis, and muscle weakness.

Thus, there is a need to develop therapeutics to enhance muscle function, such as for treating muscle-related disease and disorders.

### SUMMARY

Disclosed herein, at least in part, is a composition comprising at least four different amino acid entities. In some embodiments, the composition is capable of of one, two, three, or all of:

a) activating mTORC1;

b) activating protein synthesis and/or inhibiting protein catabolism;

c) improving, e.g., increasing, insulin sensitivity or glucose tolerance;

d) reducing inflammation; or

e) increasing or improving myogenesis or myotube growth.

In some embodiments, the protein synthesis is muscle protein synthesis. In some embodiments, the protein catabolism is muscle protein catabolism.

In some embodiments, the composition is capable of improving one or more metabolic symptoms selected from one, two, three, four, five, six, seven, or more (e.g., all) of mTORC1 activation; improved insulin sensitivity; activation of muscle protein synthesis; scavenging of reactive oxygen species (ROS); decreased inflammation; inhibition of catabolism; ammonia detoxification; and decreased fibrosis progression.

The composition can be used to improve or enhance muscle function in a subject. Thus, a method, including a dosage regimen, for treating (e.g., inhibiting, reducing, ameliorating, or preventing) muscle function and various muscle disorders, diseases, or symptoms thereof using the composition is disclosed herein.

In some embodiments, the subject has or is identified as having decreased muscle function due to aging, injury, atrophy, infection, or disease. In some embodiments, the subject has a rare muscle disease. In some embodiments, the subject has sarcopenia, muscle deterioration, decay, atrophy, cachexia, drug-induced myopathy, muscular dystrophy, or myopenia.

In some embodiments, the composition comprises a leucine (L)-amino acid entity, an arginine (R)-amino acid entity, a glutamine (Q)-amino acid entity; and an antioxidant or reactive oxygen species (ROS) scavenger (e.g., a N-acetylcysteine (NAC) entity, e.g., NAC). In some embodiments, at least one amino acid entity in the compositions is not provided as a peptide of more than 20 amino acid residues in length.

In some embodiments, the composition further comprises one or more essential amino acid (EAA)-entities. In some embodiments, the EAA-entities are chosen from one, two, three, or four of a histidine (H)-amino acid-entity, a lysine (K)-amino acid-entity, a phenylalanine (F)-amino acid-entity, and a threonine (T)-amino acid-entity.

In another aspect, a composition comprises a) a leucine (L)-amino acid entity, a arginine (R)-amino acid entity, and a glutamine (Q)-amino acid entity; and b) an antioxidant or reactive oxygen species (ROS) scavenger, e.g., a N-acetylcysteine (NAC) entity, e.g., NAC; and optionally c) an essential amino acid (EAA)-entity chosen from a histidine (H)-amino acid-entity, a lysine (K)-amino acid-entity, a phenylalanine (F)-amino acid-entity, and a threonine (T)-amino acid-entity or a combination of two, three, or four of the EAAs; provided that: d) at least one amino acid entity is not provided as a peptide of more than 20 amino acid residues in length, and optionally wherein: (i) the amino acid entity of (a) is selected from Table 2; and (ii) one or both of the R-amino acid entity and the Q-amino acid entity are present at a higher amount (wt. %) than the L-amino acid entity.

The compositions can also be used as a dietary composition, e.g., a medical food, a functional food, or a supplement.

In another aspect, the invention features a composition including free amino acids, wherein the amino acids include arginine, glutamine, N-acetylcysteine; a branched-chain amino acid chosen from one, two, or all of leucine, isoleucine, and valine; and an essential amino acid chosen from one, two, three, or all of histidine, lysine, phenylalanine, and threonine.

In some embodiments, the branched-chain amino acid is leucine, isoleucine, and valine. In some embodiments, the essential amino acid is histidine, lysine, phenylalanine, and threonine.

In some embodiments, the composition includes a ratio of branched-chain amino acids to total amino acids of about 4:7 to about 1:2.

In some embodiments, the weight (wt.) ratio of leucine, isoleucine, valine, arginine, glutamine, N-acetylcysteine, histidine, lysine, phenylalanine, and threonine is about 2.0:1.0:1.0:3.0:2.66:0.3:0.16:0.7:0.16:0.34.

In some embodiments, the total wt. of amino acids present is between about 4 g and about 80 g. In certain embodiments, the total wt. of amino acids present is about 6 g, about 18 g, about 24 g, or about 72 g.

In certain embodiments, the composition includes at least 1 g of leucine, at least 0.5 g of isoleucine, at least 0.5 g of valine, at least 1.5 g of arginine, at least 1.33 g of glutamine, at least 0.15 g of N-acetylcysteine, at least 0.08 g of histidine, at least 0.35 g of lysine, at least 0.08 g of phenylalanine, and at least 0.17 g of threonine.

In certain embodiments, the composition includes at least 3 g of leucine, at least 1.5 g of isoleucine, at least 1.5 g of valine, at least 4.5 g of arginine, at least 3.99 g of glutamine, at least 0.45 g of N-acetylcysteine, at least 0.24 g of histidine, at least 1.05 g of lysine, at least 0.24 g of phenylalanine, and at least 0.51 g of threonine.

In some embodiments, the amino acids include about 10 wt % to about 20 wt % leucine, about 5 wt % to about 15 wt % isoleucine, about 5 wt % to about 15 wt % valine, about 20 wt % to about 40 wt % arginine, about 15 wt % to about 35 wt % glutamine, about 1 wt % to about 10 wt % N-acetylcysteine, about 0.5 wt % to about 5 wt % histidine, about 3 wt % to about 8 wt % lysine, about 0.5 wt % to about 5 wt % phenylalanine, and about 1 wt % to about 8 wt % threonine.

In any of the aspects and embodiments disclosed herein, the wt. ratio of the L-amino acid entity, the R-amino acid entity, the L-glutamine or a salt thereof, and the NAC or a salt thereof is about 1-3:2-4:2-4:0.1-1.5; e.g., the wt. ratio of the L-amino acid entity, the I-amino acid entity, the V-amino acid entity, the R-amino acid entity, the L-glutamine or a salt thereof, the NAC or a salt thereof, the L-histidine or a salt thereof, the L-lysine or a salt thereof, the L-phenylalanine or a salt thereof, and the L-threonine or a salt thereof entity is about 1-3:0.5-1.5:0.5-1.5:2-4:2-4:0.1-1.5:0.1-0.5:0.2-1.0:0.1-0.5:0.2-0.7. In some embodiments, the wt. ratio of the L-amino acid entity, the R-amino acid entity, the L-glutamine or a salt thereof, and the NAC or salt thereof is about 0.5 to 3:0.5 to 4:1 to 4:0.1 to 2.5, e.g., the wt. ratio of the L-amino acid entity, the R-amino acid entity, the L-glutamine or a salt thereof, and the NAC or salt thereof is about 1:1.5:2:0.15 or about 1:1.5:2:0.3. In any of the aforesaid embodiments in this paragraph, the wt. ratio of the L-amino acid entity, the R-amino acid entity, the L-glutamine or a salt thereof, and the NAC or salt thereof is about 1:0.75:2:0.15 or about 1:0.75:2:0.3.

In any of the aspects and embodiments disclosed herein, the wt. ratio of the L-amino acid entity, the I-amino acid entity, the V-amino acid entity, the R-amino acid entity, the L-glutamine or salt thereof, and the NAC or salt thereof is about 1:0.5:0.5:1.5:2:0.15 or about 1:0.5:0.5:1.5:2:0.3.

In some embodiments, the wt. ratio of the L-amino acid entity, the R-amino acid entity, the L-glutamine or a salt thereof, and the NAC or salt thereof is about 1+/−15%:1.5+/−15%:2+/−15%:0.15+/−15% or about 1+/−15%:1.5+/−15%:2+/−15%:0.3+/−15%. In any of the aforesaid embodiments in this paragraph, the wt. ratio of the L-amino acid entity, the R-amino acid entity, the L-glutamine or a salt thereof, and the NAC or salt thereof is about 1+/−15%:0.75+/−15%:2+/−15%:0.15+/−15% or about 1+/−15%:0.75+/−15%:2+/−15%:0.3+/−15%.

In any of the aspects and embodiments disclosed herein, the wt. ratio of the L-amino acid entity, the I-amino acid entity, the V-amino acid entity, the R-amino acid entity, the L-glutamine or salt thereof, and the NAC or salt thereof is about 1+/−15%:0.5+/−15%:0.5+/−15%:1.5+/−15%:2+/−15%:0.15+/−15% or about 1+/−15%:0.5+/−15%:0.5+/−15%:1.5+/−15%:2+/−15%:0.3+/−15%.

In some embodiments, the composition further includes one or more pharmaceutically acceptable excipients.

In some embodiments, the amino acids consist of leucine, isoleucine, valine, arginine, glutamine, N-acetylcysteine, histidine, lysine, phenylalanine, and threonine.

Also provided is a method of treating physiological symptoms selected from one, two, three, four, five, six, seven, eight, nine, or more (e.g., all) of immobilization, malnutrition, fasting, aging, autophagy, reduced protein synthesis, anabolic resistance, junction integrity (e.g., neuromuscular junction integrity), insulin resistance, or an energy deficit in a subject that includes administering to a subject in need thereof an effective amount of the composition. In some embodiments, the subject has a rare muscle disease.

In some embodiments, the subject has sarcopenia. In some embodiments, the subject has muscle deterioration. In some embodiments, the subject has muscle decay. In some embodiments, the subject has muscle atrophy. In some embodiments, the subject has cachexia. In some embodiments, the subject has drug-induced myopathy. In some embodiments, the subject has muscular dystrophy. In some embodiments, the subject has myopenia. In certain embodiments, the compositions are capable of improving ventilator-induced diaphragm atrophy or ventilator-induced diaphragmatic dysfunction in the subject.

Another aspect of the invention features a method for treating physiological symptoms selected from one, two, three, four, five, six, seven, eight, nine, or more (e.g., all) of immobilization, malnutrition, fasting, aging, autophagy, reduced protein synthesis, anabolic resistance, junction integrity (e.g., neuromuscular junction integrity), insulin resistance, decreased mitochondrial biogenesis, anaplerosis, or an energy deficit that comprises administering to a subject in need thereof an effective amount of the composition of any of the foregoing aspects or embodiments.

In some embodiments, the subject has a rare muscle disease.

In some embodiments, the subject has muscle deterioration, muscle decay, muscle atrophy, cachexia, sarcopenia, drug-induced myopathy, muscular dystrophy, or myopenia.

Another aspect of the invention features a method for enhancing muscle function including administering to a subject in need thereof an effective amount of a composition of any of the foregoing aspects or embodiments.

In some embodiments, the subject has or is identified as having decreased muscle function due to aging, injury, atrophy, infection, or disease.

In some embodiments, the subject has or is identified as having muscle deterioration, muscle decay, muscle atrophy, cachexia, sarcopenia, drug-induced myopathy, muscular dystrophy, or myopenia.

In some embodiments, administration of the composition results in an improvement in one or more metabolic symptoms in the subject. In some embodiments, the improvement in one or more metabolic symptoms is selected from the following: mTORC1 activation; improved insulin sensitivity; activation of muscle protein synthesis; scavenging of reactive oxygen species (ROS); decreased inflammation; inhibition of catabolism; ammonia detoxification; and decreased fibrosis progression.

In some embodiments, administration of the composition reduces muscle atrophy in the subject.

In some embodiments, administration of the composition results in anabolism and catabolism of muscle tissue.

In some embodiments, the subject is a human.

Another aspect of the invention features a dietary composition including the composition any of the foregoing aspects or embodiments, e.g., wherein the dietary composition is chosen from a medical food, a functional food, or a supplement.

Another aspect of the invention features a composition of any of the foregoing aspects or embodiments for use as a dietary composition, e.g., wherein the dietary composition is chosen from a medical food, a functional food, or a supplement.

In some embodiments, the composition is for use in treating a subject having or identified as having decreased muscle function due to aging, injury, atrophy, infection, or disease.

In some embodiments, the subject has or is identified as having muscle deterioration, muscle decay, muscle atrophy, cachexia, sarcopenia, drug-induced myopathy, muscular dystrophy, or myopenia.

One embodiment provides a nutritional supplement, dietary formulation, functional food, medical food, food, or beverage comprising a composition described herein. Another embodiment provides a nutritional supplement, dietary formulation, functional food, medical food, food, or beverage comprising a composition described herein for use in the management of any of the diseases or disorders described herein.

One embodiment provides a method of maintaining or improving muscle health, muscle function, muscle functional performance, or muscle strength, comprising administering to a subject an effective amount of a composition described herein. Another embodiment provides a method of providing nutritional support or supplementation to a subject suffering from muscle atrophy comprising administering to the subject an effective amount of a composition described herein. Yet another embodiment provides a method of providing nutritional support or supplementation that aids in the management of muscle atrophy to a subject comprising administering to the subject in need thereof an effective amount of a composition described herein.

Additional features and embodiments of the present invention include one or more of the following:

Another aspect of the invention features a composition comprising:

a) a L-amino acid entity chosen from L-leucine or a salt thereof, or β-hydroxy-β-methybutyrate (HMB) or a salt thereof, or a combination of L-leucine or a salt thereof and HMB or a salt thereof;

b) an R-amino acid entity chosen from L-arginine or a salt thereof, ornithine or a salt thereof, or creatine or a salt thereof or a combination of two or three of L-arginine or a salt thereof, ornithine or a salt thereof, or creatine or a salt thereof; and

c) L-glutamine or a salt thereof;

d) N-acetylcysteine (NAC) or a salt thereof; and

e) an EAA chosen from L-histidine or a salt thereof, L-lysine or a salt thereof, L-phenylalanine or a salt thereof, or L-threonine or a salt thereof, or a combination of two, three, or four of the EAAs. In some embodiments of any of the compositions or methods disclosed herein, the L-leucine is provided as part of a dipeptide comprising L-leucine, or a salt thereof, or a tripeptide comprising L-leucine, or a salt thereof.

In some embodiments of any of the compositions or methods disclosed herein, the L-arginine is provided as part of a dipeptide comprising L-arginine, or a salt thereof, or a tripeptide comprising L-arginine, or a salt thereof.

In some embodiments of any of the compositions or methods disclosed herein, the L-glutamine is provided as part of a dipeptide comprising L-glutamine, or a salt thereof, or a tripeptide comprising L-glutamine, or a salt thereof.

In some embodiments of any of the compositions or methods disclosed herein, the NAC is provided as part of a dipeptide comprising NAC, or a salt thereof, or a tripeptide comprising NAC, or a salt thereof.

In some embodiments of any of the compositions or methods disclosed herein, the L-histidine is provided as part of a dipeptide comprising L-histidine, or a salt thereof, or a tripeptide comprising L-histidine, or a salt thereof.

In some embodiments of any of the compositions or methods disclosed herein, the L-lysine is provided as part of a dipeptide comprising L-lysine, or a salt thereof, or a tripeptide comprising L-lysine, or a salt thereof.

In some embodiments of any of the compositions or methods disclosed herein, the L-phenylalanine is provided as part of a dipeptide comprising L-phenylalanine, or a salt thereof, or a tripeptide comprising L-phenylalanine, or a salt thereof.

In some embodiments of any of the compositions or methods disclosed herein, the L-threonine is provided as part of a dipeptide comprising L-threonine, or a salt thereof, or a tripeptide comprising L-threonine, or a salt thereof.

In some embodiments of any of the compositions or methods disclosed herein, one, two, three, or four of methionine (M), tryptophan (W), valine (V), or cysteine (C) is absent, or if present, is present at a percentage weight of the composition (wt. %) of less than 10%. In some embodiments of any of the compositions or methods disclosed herein, the total wt. % of (a)-(e) is greater than the total wt. % of any other amino acid entity in the composition.

In some embodiments of any of the compositions or methods disclosed herein, one, two, three, four, or five of the amino acids in (a)-(e) is provided as a dipeptide or tripeptide, e.g., in an amount of at least 10 wt. % of the composition. In some embodiments of any of the compositions or methods disclosed herein, the dipeptide is a homodipeptide or heterodipeptide of any of the amino acids in (a)-(e), e.g., one, two, three, or four of (a)-(e) is a homodipeptide or heterodipeptide. In some embodiments of any of the compositions or methods disclosed herein, the tripeptide is a homotripeptide or heterotripeptide of any of (a)-(e), e.g., one, two, three, or four of (a)-(e) is a homotripeptide or heterotripeptide.

In some embodiments of any of the compositions or methods disclosed herein, (a) is a L-amino acid entity dipeptide or a salt thereof (e.g., a L-leucine dipeptide or a salt thereof). In some embodiments of any of the compositions or methods disclosed herein, (a) is a homodipeptide. In some embodiments of any of the compositions or methods disclosed herein, (a) is a heterodipeptide, e.g., Ala-Leu.

In some embodiments of any of the compositions or methods disclosed herein, (b) is a L-arginine dipeptide or a salt thereof. In some embodiments of any of the compositions or methods disclosed herein, (b) is a homodipeptide. In some embodiments, (b) is a heterodipeptide, e.g., Ala-Arg.

In some embodiments of any of the compositions or methods disclosed herein, (c) is a L-glutamine dipeptide or a salt thereof. In some embodiments of any of the compositions or methods disclosed herein, (c) is a homodipeptide, e.g., Gln-Gln. In some embodiments, (c) is a heterodipeptide, e.g., Ala-Gln.

In some embodiments of any of the compositions or methods disclosed herein:

f) a wt. % of the R-amino acid entity in the composition is greater than the wt. % of the L-glutamine or a salt thereof;

g) the wt. % of the L-glutamine or a salt thereof in the composition is greater than the wt. % of the L-amino acid entity;

h) the wt. % of the R-amino acid entity in the composition is greater than the wt. % of the L-amino acid entity;

i) the wt. % of the R-amino acid entity in the composition is greater than the wt. % of the EAA, or the combination of two, three, or four of the EAAs;

j) the wt. % of the L-glutamine or a salt thereof in the composition is greater than the wt. % of the EAA or the combination of two, three, or four of the EAAs;

k) the wt. % of the L-amino acid entity in the composition is greater than the wt. % of the EAA or the combination of two, three, or four of the EAAs; or

l) a combination of two, three, four, five, or six of (f)-(k).

In some embodiments of any of the compositions or methods disclosed herein, the wt. % of the R-amino acid entity in the composition is at least 2% greater than the wt. % of the L-glutamine or a salt thereof, e.g., the wt. % of the L-glutamine or a salt thereof is at least 3%, 4%, 5%, 6%, 7%, 8%, 9%, or 10% greater than the wt. % of the R-amino acid entity.

In some embodiments of any of the compositions or methods disclosed herein, the wt. % of the L-glutamine or a salt thereof in the composition is at least 10% greater than the wt. % of the L-amino acid entity, e.g., the wt. % of the L-glutamine or a salt thereof in the composition is at least 12%, 15%, 20%, 22%, or 25% greater than the wt. % of the L-amino acid entity.

In some embodiments of any of the compositions or methods disclosed herein, the wt. % of the R-amino acid entity in the composition is at least 10% greater than the wt. % of the L-amino acid entity, e.g., the wt. % of the R-amino acid entity in the composition is at least 15%, 20%, 25%, or 30% greater than the wt. % of the L-amino acid entity.

In some embodiments of any of the compositions or methods disclosed herein, the wt. % of the R-amino acid entity in the composition is at least 25% greater than the wt. % of the EAA or the combination of two, three, or four of the EAAs, e.g., the wt. % of the R-amino acid entity in the composition is at least 20%, 30%, 40%, or 50% greater than the wt. % of the EAA or the combination of two, three, or four of the EAAs.

In some embodiments of any of the compositions or methods disclosed herein, the wt. % of the L-glutamine or a salt thereof in the composition is at least 25% greater than the wt. % of the EAA or the combination of two, three, or four of the EAAs, e.g., the wt. % of the L-glutamine or a salt thereof in the composition is at least 20%, 30%, 40%, or 50% greater than the wt. % of the EAA or the combination of two, three, or four of the EAAs.

In some embodiments of any of the compositions or methods disclosed herein, the wt. % of the L-amino acid entity in the composition is at least 10% greater than the wt. % of the EAA or the combination of two, three, or four of the EAAs, e.g., the wt. % of the L-glutamine or a salt thereof in the composition is at least 12%, 15%, 20%, 22%, or 25% greater than the wt. % of the EAA or the combination of two, three, or four of the EAAs.

In some embodiments of any of the compositions or methods disclosed herein:

m) the ratio of the L-amino acid entity to the R-amino acid entity is at least 1:4, or at least 2:5, and not more than 3:4, e.g., the ratio of L-amino acid entity to R-amino acid entity is about 2:3;

n) the ratio of the L-amino acid entity to the L-glutamine or a salt thereof is at least 1:4, or least 1:3, and not more than 3:4, e.g., the ratio of the L-amino acid entity to the L-glutamine or a salt thereof is about 2:3;

o) the ratio of the L-glutamine or a salt thereof to the R amino acid entity is at least 1:2, or least 3:4, and not more than 11:12, e.g., the ratio of the L-glutamine or a salt thereof to the R-amino acid entity is about 8:9;

p) the ratio of the EAA, or the combination of two, three, or four of the EAAs, to the L-amino acid entity is at least 1:4, or at least 2:5, and not more than 3:4, e.g., the ratio of the EAA, or the combination of two, three, or four of the EAAs, to the L-amino acid entity is about 2:3;

q) the ratio of the EAA, or the combination of two, three, or four of the EAAs, to the L-glutamine or a salt thereof is at least 1:4, or at least 2:5, and not more than 3:4, e.g., the ratio of the EAA, or the combination of two, three, or four of the EAAs, to the L-glutamine or a salt thereof is about 1:2;

r) the ratio of the EAA to the R-amino acid entity is at least 1:5, or at least 1:3, and not more than 2:3, e.g., the ratio of the EAA, or the combination of two, three, or four of the EAAs, to the R-amino acid entity is about 4:9; or

s) a combination of two, three, four, five, or six of (m)-(r).

In some embodiments of any of the compositions or methods disclosed herein, the composition further comprises one or both of an isoleucine (I)-amino acid-entity and a valine (V)-amino acid-entity, e.g., both the I-amino acid-entity and the V-amino acid-entity are present.

In certain embodiments:

t) the wt. % of the L-amino acid-entity in the composition is greater than or equal to the wt. % of the I-amino acid-entity and the V-amino acid-entity in combination;

u) the wt. % of the L-amino acid-entity, the I-amino acid-entity, and the V-amino acid-entity in combination in the composition is greater than or equal to the wt. % of the L-glutamine or a salt thereof;

v) the wt. % of the L-amino acid-entity, the I-amino acid-entity, and the V-amino acid-entity in combination in the composition is less than the wt. % of the R-amino acid entity;

w) the wt. % of the R-amino acid entity and the L-glutamine or a salt thereof in the composition is greater than the wt. % of the L-amino acid-entity, the I-amino acid-entity, and the V-amino acid-entity in combination;

x) the wt. % of the L-amino acid-entity, the I-amino acid-entity, and the V-amino acid-entity in combination is greater than the EAA, or the combination of two, three, or four of the EAAs, in the composition;

y) the wt. % of the I-amino acid-entity in combination with the L-amino acid entity or the V-amino acid-entity is greater than the EAA, or the combination of two, three, or four of the EAAs, in the composition;

z) the wt. % of the V-amino acid entity is greater than the EAA, or the combination of two, three, or four of the EAAs, in the composition; or

aa) a combination of two, three, four, five, six, or seven of (t)-(z).

In some embodiments of any of the compositions or methods disclosed herein:

bb) the wt. % of the R-amino acid entity, the L-glutamine or a salt thereof, and the NAC or a salt thereof is at least 30% of the composition, or at least 40% of the composition, but not more than 70% of the composition;

cc) the wt. % of the NAC or a salt thereof is at least 1%, or at least 2%, but not more than 10% of the composition;

dd) the wt. % of the L-amino acid-entity, the I-amino acid-entity, and the V-amino acid-entity in combination is at least 20%, or at least 25%, but not more than 60% of the composition;

ee) the wt. % of the R-amino acid entity, the L-glutamine or a salt thereof, and the NAC or a salt thereof is at least 40%, or at least 50%, but not more than 80% of the composition;

ff) the wt. % of the EAA, or the combination of two, three, or four of the EAAs, in the composition is at least 5%, or at least 10%, but not more than 25%, e.g., the wt. % of the EAA, or the combination of two, three, or four of the EAAs, is about 12% or about 14%; or

gg) a combination of two, three, four, or five of (bb)-(ff).

In certain embodiments:

hh) the ratio of the L-amino acid entity to the I-amino acid entity is at least 3:2, or at least 7:4, and not more than 5:2 or not more than 3:1, e.g., the ratio of the L-amino acid entity to the I-amino acid entity is about 2:1;

ii) the ratio of L-amino acid entity to V-amino acid entity is at least 3:2, or at least 7:4, and not more than 5:2 or not more than 3:1, e.g., the ratio of L to V is about 2:1;

jj) the ratio of the L-amino acid entity to the R-amino acid entity is greater than 1:3, greater than 1:2, and less than 3:4, e.g., the ratio of the L-amino acid entity to the R-amino acid entity is about 2:3;

kk) the ratio of the L-amino acid entity to the L-glutamine or a salt thereof is greater than 1:4, greater than 3:8, and less than 5:6, or less than 6:7, e.g., the ratio of the L-amino acid entity to the L-glutamine or a salt thereof is about 3:4;

ll) the ratio of the EAA, or the combination of two, three, or four of the EAAs, to the L-amino acid is greater than 1:4, greater than 3:8, and less than 3:4, or less than 5:6, e.g., the ratio of the EAA, or the combination of two, three, or four of the EAAs, to the L-amino acid entity is about 2:3; or

mm) a combination of two, three, four, or five of (hh)-(ll).

In some embodiments of any of the compositions or methods disclosed herein:

nn) the ratio of the I-amino acid entity to the V-amino acid entity is at least 0.5:1, or at least 0.75:1, and not more than 1.5 to 1 or not more than 2:1, e.g., the ratio of the L-amino acid entity to the I-amino acid entity is about 1:1;

oo) the ratio of the I-amino acid entity to the R-amino acid entity is at least 1:6, or at least 0.75:3, and not more than 2:3, or not more than 1.5:3, e.g., the ratio of the L-amino acid entity to the I-amino acid entity is about 1:3;

pp) the ratio of the I-amino acid entity to the L-glutamine or a salt thereof is at least 1:8, or at least 1:4, and not more than 3:4, or not more than 1:2, e.g., the ratio of the L-amino acid entity to the L-glutamine or a salt thereof is about 3:8;

qq) the ratio of the I-amino acid to the EAA, or the combination of two, three, or four of the EAAs, to is greater than 1:3, greater than 1:2, and less than 5:6, or less than 6:7, e.g., the ratio of the I-amino acid entity to the EAA, or the combination of two, three, or four of the EAAs, is about 3:4; or

rr) a combination of two, three, or four of (nn)-(qq).

In some embodiments of any of the compositions or methods disclosed herein:

ss) the ratio of the L-amino acid entity to the V-amino acid entity is at least 3:2, or at least 7:4, and not more than 3:1 or not more than 4:1, e.g., is the ratio of the L-amino acid entity to the V-amino acid entity is about 2:1;

tt) the ratio of the L-amino acid entity to the R-amino acid entity is greater than 1:3, greater than 3:6, and less than 3:4, e.g., the ratio of the L-amino acid entity to the R-amino acid entity is about 2:3;

uu) the ratio of the L-amino acid entity to the L-glutamine or a salt thereof is greater than 1:4, greater than 1:2 and less than 5:6, or less than 6:7, e.g., the ratio of the L-amino acid entity to the L-glutamine or a salt thereof is about 3:4;

vv) the ratio of the I-amino acid to the EAA, or the combination of two, three, or four of the EAAs, to is greater than 1:3, greater than 1:2, and less than 5:6, or less than 6:7, e.g., the ratio of the I-amino acid entity to the EAA, or the combination of two, three, or four of the EAAs, is about 3:4; or

ww) a combination of two, three, or four of (ss)-(vv).

In some embodiments of any of the compositions or methods disclosed herein:

xx) the ratio of the V-amino acid entity to the L-glutamine or a salt thereof is at least 1:8, or at least 1:4, and not more than 3:4, or not more than 1:2, e.g., the ratio of the L-amino acid entity to the L-glutamine or a salt thereof is about 3:8;

yy) the ratio of the V-amino acid entity to the R-amino acid entity is at least 1:9, or at least 2:9, and not more than 2:3, or not more than 1:2, e.g., the ratio of the V-amino acid entity to the R-amino acid entity is 1:3;

zz) the ratio of the L-amino acid-entity, the I-amino acid-entity, and the V-amino acid-entity in combination to the R-amino acid entity, L-glutamine or a salt thereof, and NAC or a salt thereof is at least 1:4, or at least 1:3, and not more than 7:9, or not more than 8:9, e.g., the ratio is about 6:9;

aaa) the ratio of the EAA, or the combination of two, three, or four of the EAAs, to the L-amino acid-entity, the I-amino acid-entity, and the V-amino acid-entity in combination to is at least 1:5, or at least 1:4, and not more than 2:3, or not more than 3:4, e.g., the ratio is about 1:3; or

bbb) a combination of two, three, or four of (xx)-(aaa).

In some embodiments of any of the compositions or methods disclosed herein:

ccc) a wt. % of the L-amino acid entity in the composition is greater than the wt. % of the NAC or a salt thereof;

ddd) a wt. % of the R-amino acid entity in the composition is greater than the wt. % of the NAC or a salt thereof;

eee) a wt. % of the L-glutamine or a salt thereof in the composition is greater than the wt. % of the NAC or a salt thereof; or

fff) a combination of two or three of (ccc)-(eee).

In some embodiments of any of the compositions or methods disclosed herein, at least one of (a)-(d) is a free amino acid, e.g., two, three, or four of (a)-(d) are a free amino acid, e.g., at least 50 wt. % of the total wt. of the composition is one or more amino acid entities in free form.

In some embodiments of any of the compositions or methods disclosed herein, at least one of (a)-(d) is in a salt form, e.g., one, two, three, or four of (a)-(d) is in a salt form, e.g., at least 10 wt. % of the total wt. of the composition is one or more amino acid entities in salt form.

In some embodiments of any of the compositions or methods disclosed herein, the composition is capable of one, two, three, four or all of:

a) activating mTORC1;

b) activating protein synthesis and/or inhibiting protein catabolism;

c) improving, e.g., increasing, insulin sensitivity or glucose tolerance;

d) reducing inflammation; or

e) improving or increasing myogenesis.

In some embodiments of any of the compositions or methods disclosed herein, the wt. ratio of the L-amino acid entity, the R-amino acid entity, the L-glutamine or a salt thereof, and the NAC or a salt thereof is about 1-3:2-4:2-4:0.1-1.5; e.g., the wt. ratio of the L-amino acid entity, the I-amino acid entity, the V-amino acid entity, the R-amino acid entity, the L-glutamine or a salt thereof, the NAC or a salt thereof, the L-histidine or a salt thereof, the L-lysine or a salt thereof, the L-phenylalanine or a salt thereof, and the L-threonine or a salt thereof entity is about 1-3:0.5-1.5:0.5-1.5:2-4:2-4:0.1-1.5:0.1-0.5:0.2-1.0:0.1-0.5:0.2-0.7.

In some embodiments of any of the compositions or methods disclosed herein, the wt. ratio of the L-amino acid entity, the R-amino acid entity, the L-glutamine or a salt thereof, and the NAC or salt thereof is about 0.5 to 3:0.5 to 4:1 to 4:0.1 to 2.5, e.g., the wt. ratio of the L-amino acid entity, the R-amino acid entity, the L-glutamine or a salt thereof, and the NAC or salt thereof is about 1:1.5:2:0.15 or about 1:1.5:2:0.3. In any of the aforesaid embodiments in this paragraph, the wt. ratio of the L-amino acid entity, the R-amino acid entity, the L-glutamine or a salt thereof, and the NAC or salt thereof is about 1:0.75:2:0.15 or about 1:0.75:2:0.3.

In some embodiments of any of the compositions or methods disclosed herein, the wt. ratio of the L-amino acid entity, the I-amino acid entity, the V-amino acid entity, the R-amino acid entity, the L-glutamine or salt thereof, and the NAC or salt thereof is about 1:0.5:0.5:1.5:2:0.15 or about 1:0.5:0.5:1.5:2:0.3.

In some embodiments of any of the compositions or methods disclosed herein, the wt. ratio of the L-amino acid entity, the I-amino acid entity, the V-amino acid entity, the R-amino acid entity, the L-glutamine or salt thereof, and the NAC or salt thereof is about 1+/−15%:0.5+/−15%:0.5+/−15%:1.5+/−15%:2+/−15%:0.15+/−15% or about 1+/−15%:0.5+/−15%:0.5+/−15%:1.5+/−15%:2+/−15%:0.3+/−15%.

In some embodiments of any of the compositions or methods disclosed herein, the composition comprises about 0.5 g to about 10 g of the L-amino acid entity, about 0.25 g to about 5 g of the I-amino acid entity, about 0.25 g to about 5 g of the V-amino acid entity, about 0.5 g to about 20 g of the R-amino acid entity, about 1 g to about 20 g of the L-glutamine or a salt thereof, and about 0.1 g to about 5 g of the NAC or a salt thereof, e.g., the composition comprises about 1 g of the L-amino acid entity, about 0.5 g of the I-amino acid entity, about 0.5 g of V-amino acid entity, about 1.5 g of R-amino acid entity, about 2 g of L-glutamine or a salt thereof, and about 0.15 g or about 0.3 g of NAC or a salt thereof. In some embodiments of any of the compositions or methods disclosed herein, the composition comprises about 0.15 g of NAC. In some embodiments of any of the compositions or methods disclosed herein, the composition comprises about 0.3 g of NAC.

In some embodiments of any of the compositions or methods disclosed herein, the composition comprises about 1 g of the L-amino acid entity, about 0.5 g of the I-amino acid entity, about 0.5 g of V-amino acid entity, about 0.75 g of R-amino acid entity, about 2 g of L-glutamine or a salt thereof, and about 0.15 g or about 0.3 g of NAC or a salt thereof. In embodiments, the composition comprises about 0.15 g of NAC. In some embodiments of any of the compositions or methods disclosed herein, the composition comprises about 0.3 g of NAC. In some embodiments of any of the compositions or methods disclosed herein, the composition comprises about 4 g of the L-amino acid entity, about 2 g of the I-amino acid entity, about 1 g of V-amino acid entity, about 3 g of R-amino acid entity, about 4 g of L-glutamine or a salt thereof, and about 0.9 g of NAC or a salt thereof.

In some embodiments of any of the compositions or methods disclosed herein, the composition comprises about 0.5 g to about 15 g of the L-amino acid entity, about 0.25 g to about 10 g of the I-amino acid entity, about 0.25 g to about 10 g of the V-amino acid entity, about 0.5 to about 25 g of the R-amino acid entity, about 0.5 g to about 20 g of the L-glutamine or a salt thereof, about 0.1 to about 5 g the NAC or a salt thereof, about 0.05 g to about 3 g of the L-histidine or a salt thereof, about 0.05 to about 6 g of the L-lysine or a salt thereof, about 0.04 to about 2 g of the L-phenylalanine or a salt thereof, and about 0.08 to about 4 g of the L-threonine or a salt thereof entity; e.g., about 1 g of the L-amino acid entity, about 0.5 g of the I-amino acid entity, about 0.5 g of the V-amino acid entity, about 1.5 g or about 1.81 of the R-amino acid entity, about 1.33 g of the L-glutamine or a salt thereof, about 0.15 g or about 0.3 g of the NAC or a salt thereof, about 0.08 g of the L-histidine or a salt thereof, about 0.35 g of the L-lysine or a salt thereof, about 0.08 g of the L-phenylalanine or a salt thereof, and about 0.17 g of the L-threonine or a salt thereof.

In some embodiments of any of the compositions or methods disclosed herein:

a) L-Leucine or a salt thereof;

b) L-Isoleucine or a salt thereof;

c) L-Valine or a salt thereof;

d) L-Arginine or a salt thereof;

e) L-Glutamine or a salt thereof;

f) NAC or a salt thereof; and

g) L-histidine or a salt thereof, L-lysine or a salt thereof, L-phenylalanine or a salt thereof, and L-threonine or a salt thereof.

In some embodiments of any of the compositions or methods disclosed herein, L-Leucine is provided as part of a dipeptide comprising L-Leucine, or a salt thereof, or a tripeptide comprising L-Leucine, or a salt thereof.

In some embodiments of any of the compositions or methods disclosed herein, L-Isoleucine is provided as part of a dipeptide comprising L-Isoleucine, or a salt thereof, or a tripeptide comprising L-Isoleucine, or a salt thereof.

In some embodiments of any of the compositions or methods disclosed herein, L-Valine is provided as part of a dipeptide comprising L-Valine, or a salt thereof, or a tripeptide comprising L-Valine, or a salt thereof.

In some embodiments of any of the compositions or methods disclosed herein, L-Arginine is provided as part of a dipeptide comprising L-Arginine, or a salt thereof, or a tripeptide comprising L-Arginine, or a salt thereof.

In some embodiments of any of the compositions or methods disclosed herein, L-Glutamine is provided as part of a dipeptide comprising L-Glutamine, or a salt thereof, or a tripeptide comprising L-Glutamine, or a salt thereof.

In some embodiments of any of the compositions or methods disclosed herein, NAC is provided as a part of a dipeptide comprising NAC, or a salt thereof, or a tripeptide comprising NAC, or a salt thereof.

In some embodiments of any of the compositions or methods disclosed herein, the composition comprises a combination of 4 to 20 different amino acid entities, e.g., a combination of 5 to 15 different amino acid entities.

In some embodiments of any of the compositions or methods disclosed herein, at least two, three, four, or more amino acid entities are not comprised in a peptide of more than 4, 5, 6, 7, 8, 9, 10, 11, 12, 13, 14, 15, 16, 17, 18, 19, or 20 amino acid residues in length.

Another aspect of the invention features a method for improving muscle function, wherein the method comprises administering to a subject in need thereof an effective amount of a composition comprising:

a) a L-amino acid entity chosen from L-leucine or a salt thereof, or β-hydroxy-β-methybutyrate (HMB) or a salt thereof;

b) an R-amino acid entity chosen from L-arginine or a salt thereof, ornithine or a salt thereof, or creatine or a salt thereof; and

c) L-glutamine or a salt thereof;

d) N-acetylcysteine (NAC) or a salt thereof; and

e) an EAA chosen from L-histidine or a salt thereof, L-lysine or a salt thereof, L-phenylalanine or a salt thereof, or L-threonine or a salt thereof or a combination of two, three, or four of the EAAs.

In some embodiments of any of the compositions or methods disclosed herein, the L-leucine is provided as part of a dipeptide comprising L-leucine, or a salt thereof, or a tripeptide comprising L-leucine, or a salt thereof.

In some embodiments of any of the compositions or methods disclosed herein, the L-arginine is provided as part of a dipeptide comprising L-arginine, or a salt thereof, or a tripeptide comprising L-arginine, or a salt thereof.

In some embodiments of any of the compositions or methods disclosed herein, the L-glutamine is provided as part of a dipeptide comprising L-glutamine, or a salt thereof, or a tripeptide comprising L-glutamine, or a salt thereof.

In some embodiments of any of the compositions or methods disclosed herein, the NAC is provided as part of a dipeptide comprising NAC, or a salt thereof, or a tripeptide comprising NAC, or a salt thereof.

In some embodiments of any of the compositions or methods disclosed herein, the L-histidine is provided as part of a dipeptide comprising L-histidine, or a salt thereof, or a tripeptide comprising L-histidine, or a salt thereof. In some embodiments, the L-lysine is provided as part of a dipeptide comprising L-lysine, or a salt thereof, or a tripeptide comprising L-lysine, or a salt thereof.

In some embodiments of any of the compositions or methods disclosed herein, the L-phenylalanine is provided as part of a dipeptide comprising L-phenylalanine, or a salt thereof, or a tripeptide comprising L-phenylalanine, or a salt thereof. In some embodiments, the L-threonine is provided as part of a dipeptide comprising L-threonine, or a salt thereof, or a tripeptide comprising L-threonine, or a salt thereof.

Another aspect of the invention features a method for treating one or more symptoms selected from immobilization, malnutrition, fasting, aging, autophagy, reduced protein synthesis, anabolic resistance, junction integrity, insulin resistance, decreased mitochondrial biogenesis, anaplerosis, or an energy deficit, wherein the method comprises administering to a subject in need thereof an effective amount of a composition comprising:

a) a L-amino acid entity chosen from L-leucine or a salt thereof, or β-hydroxy-β-methybutyrate (HMB) or a salt thereof;

b) an R-amino acid entity chosen from L-arginine or a salt thereof, ornithine or a salt thereof, or creatine or a salt thereof; and

c) L-glutamine or a salt thereof;

d) N-acetylcysteine (NAC) or a salt thereof; and

e) an EAA chosen from L-histidine or a salt thereof, L-lysine or a salt thereof, L-phenylalanine or a salt thereof, or L-threonine or a salt thereof or a combination of two, three, or four of the EAAs.

In some embodiments of any of the compositions or methods disclosed herein, the L-leucine is provided as part of a dipeptide comprising L-leucine, or a salt thereof, or a tripeptide comprising L-leucine, or a salt thereof.

In some embodiments of any of the compositions or methods disclosed herein, the L-arginine is provided as part of a dipeptide comprising L-arginine, or a salt thereof, or a tripeptide comprising L-arginine, or a salt thereof.

In some embodiments of any of the compositions or methods disclosed herein, the L-glutamine is provided as part of a dipeptide comprising L-glutamine, or a salt thereof, or a tripeptide comprising L-glutamine, or a salt thereof.

In some embodiments of any of the compositions or methods disclosed herein, the NAC is provided as part of a dipeptide comprising NAC, or a salt thereof, or a tripeptide comprising NAC, or a salt thereof.

In some embodiments of any of the compositions or methods disclosed herein, the L-histidine is provided as part of a dipeptide comprising L-histidine, or a salt thereof, or a tripeptide comprising L-histidine, or a salt thereof.

In some embodiments of any of the compositions or methods disclosed herein, the L-lysine is provided as part of a dipeptide comprising L-lysine, or a salt thereof, or a tripeptide comprising L-lysine, or a salt thereof.

In some embodiments of any of the compositions or methods disclosed herein, the L-phenylalanine is provided as part of a dipeptide comprising L-phenylalanine, or a salt thereof, or a tripeptide comprising L-phenylalanine, or a salt thereof.

In some embodiments of any of the compositions or methods disclosed herein, the L-threonine is provided as part of a dipeptide comprising L-threonine, or a salt thereof, or a tripeptide comprising L-threonine, or a salt thereof.

Another aspect of the invention features a method of improving or increasing myogenesis, wherein the method comprises administering to a subject in need thereof an effective amount of a composition comprising:

a) a L-amino acid entity chosen from L-leucine or a salt thereof, or β-hydroxy-β-methybutyrate (HMB) or a salt thereof;

b) an R-amino acid entity chosen from L-arginine or a salt thereof, ornithine or a salt thereof, or creatine or a salt thereof; and

c) L-glutamine or a salt thereof;

d) N-acetylcysteine (NAC) or a salt thereof; and

e) an EAA chosen from L-histidine or a salt thereof, L-lysine or a salt thereof, L-phenylalanine or a salt thereof, or L-threonine or a salt thereof or a combination of two, three, or four of the EAAs.

In some embodiments of any of the compositions or methods disclosed herein, the L-leucine is provided as part of a dipeptide comprising L-leucine, or a salt thereof, or a tripeptide comprising L-leucine, or a salt thereof.

In some embodiments of any of the compositions or methods disclosed herein, the L-arginine is provided as part of a dipeptide comprising L-arginine, or a salt thereof, or a tripeptide comprising L-arginine, or a salt thereof.

In some embodiments of any of the compositions or methods disclosed herein, the L-glutamine is provided as part of a dipeptide comprising L-glutamine, or a salt thereof, or a tripeptide comprising L-glutamine, or a salt thereof.

In some embodiments of any of the compositions or methods disclosed herein, the NAC is provided as part of a dipeptide comprising NAC, or a salt thereof, or a tripeptide comprising NAC, or a salt thereof.

In some embodiments of any of the compositions or methods disclosed herein, the L-histidine is provided as part of a dipeptide comprising L-histidine, or a salt thereof, or a tripeptide comprising L-histidine, or a salt thereof.

In some embodiments of any of the compositions or methods disclosed herein, the L-lysine is provided as part of a dipeptide comprising L-lysine, or a salt thereof, or a tripeptide comprising L-lysine, or a salt thereof.

In some embodiments of any of the compositions or methods disclosed herein, the L-phenylalanine is provided as part of a dipeptide comprising L-phenylalanine, or a salt thereof, or a tripeptide comprising L-phenylalanine, or a salt thereof.

In some embodiments of any of the compositions or methods disclosed herein, the L-threonine is provided as part of a dipeptide comprising L-threonine, or a salt thereof, or a tripeptide comprising L-threonine, or a salt thereof.

In some embodiments, e.g., of any of the methods described herein, the subject has a disease or disorder selected from the group consisting of a rare muscle disease, muscle atrophy, sarcopenia, muscle deterioration, muscle decay, cachexia, drug-induced myopathy, muscular dystrophy, myopenia, muscle weakness, perceived muscle weakness, ICU-acquired myopathy, burns-related myopathy, a neuromuscular disorder, ventilator-induced diaphragmatic dystrophy, ventilator-induced diaphragmatic dysfunction, hyponatremia, hypokalemia, a calcium deficiency, hypercalcemia, amyotrophic lateral sclerosis, and a bone weakness disease.

In some embodiments, e.g., of any of the methods described herein, the subject has or is identified as having decreased muscle function due to aging, injury, muscle atrophy, infection, disease, stroke, or a fracture or other trauma.

In some embodiments, e.g., of any of the methods described herein, the subject has had a rotator cuff surgery, knee surgery, hip surgery, joint replacement, injury repair surgery, or has worn a cast prior to administration of the composition.

In some embodiments, e.g., of any of the methods described herein, the subject is treated with a composition, e.g., any composition as described herein.

### BRIEF DESCRIPTION OF THE DRAWINGS

FIG. 1 depicts the symptoms of patients in need of muscle enhancement, such as patients with muscle atrophy, prior to administration of a composition comprising amino acid entities as described herein (top) and the improvement in patients in need of muscle enhancement after administration of the composition (bottom).

FIGS. 2A and 2B are graphs showing the lean leg mass (kg) of the leg of subjects administered the amino acid composition or placebo prior to and after undergoing immobilization. Data represent mean+/−S.E.M.

FIGS. 3A and 3B are graphs showing the max torque by strength assessment of the leg of subjects administered the amino acid composition or placebo prior to and after undergoing immobilization. Data represent mean+/−S.E.M.

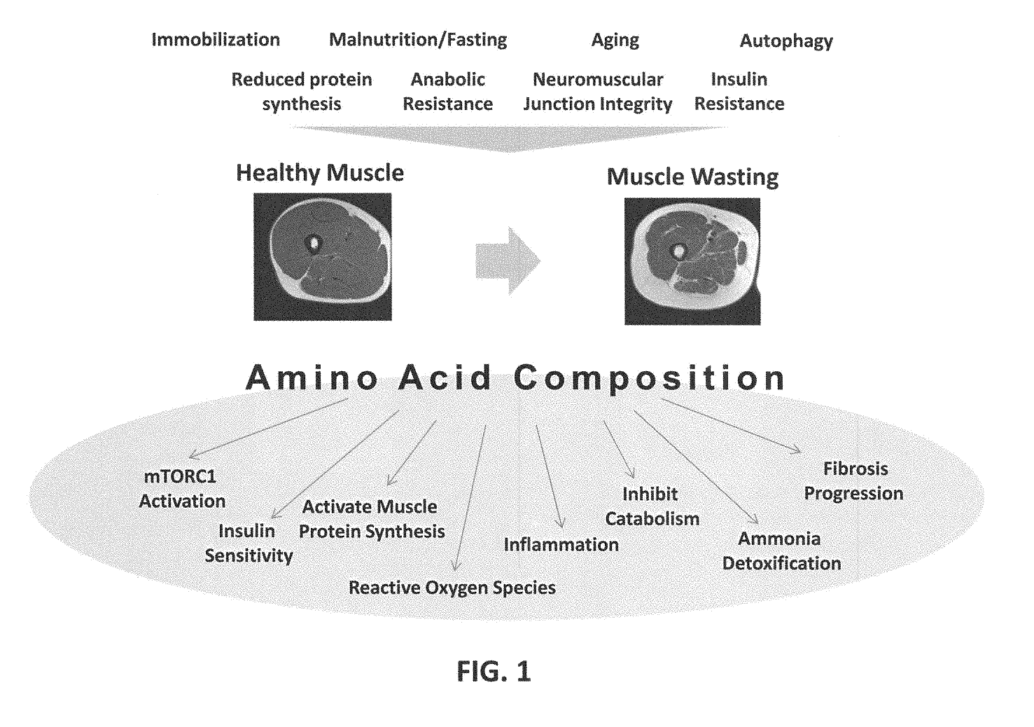

### DETAILED DESCRIPTION

The present invention provides, at least in part, methods and compositions comprising at least four different amino acid entities. In some embodiments, the composition is capable of one, two, three, or all of:

a) activating mTORC1;

b) activating protein synthesis and/or inhibiting protein catabolism;

c) improving, e.g., increasing, insulin sensitivity or glucose tolerance;

d) reducing inflammation; or

e) improving, e.g., increasing, myogenesis or myotube growth.

In some embodiments, at least one amino acid entity in the compositions is not provided as a peptide of more than 20 amino acid residues in length.

In some embodiments, the composition comprises a leucine (L)-amino acid entity, an arginine (R)-amino acid entity, a glutamine (Q)-amino acid entity; and an antioxidant or reactive oxygen species (ROS) scavenger (e.g., a N-acetylcysteine (NAC) entity, e.g., NAC). In some embodiments, at least one amino acid entity is not a peptide of more than 20 amino acid residues in length.

In some embodiments, the composition further comprises one or more essential amino acid (EAA)-entities. In some embodiments, the EAA-entities are chosen from one, two, three, or more (e.g., all) of a histidine (H)-amino acid-entity, a lysine (K)-amino acid-entity, a phenylalanine (F)-amino acid-entity, and a threonine (T)-amino acid-entity.

In some embodiments, the composition is capable of improving one or more physiological symptoms selected from one, two, three, four, five, six, seven, eight, nine, ten, or more (e.g., all) of immobilization, malnutrition, fasting, aging, autophagy, reduced protein synthesis, anabolic resistance, neuromuscular junction integrity, insulin resistance, decreased mitochondrial biogenesis, anaplerosis, myogenesis, or an energy deficit.

The composition can be administered to a subject to provide a beneficial effect in one or both of improving muscle function or treating (e.g., reversing, reducing, ameliorating, or preventing) a muscle disease or disorder. In some embodiments, the composition can be administered to treat (e.g., reverse, reduce, ameliorate, or prevent) a subject having or identified as having decreased muscle function due to aging, injury, atrophy, infection, or disease. In some embodiments, administration of the composition results in an improvement in one, two, or more of strength, stamina, or endurance in a subject, e.g., in a human. In some embodiments, administration of the composition results in an improvement, e.g., an increase, in one, two, or more of muscle cross sectional area, fiber quality, and lean muscle mass in a subject, e.g., in a human.

In some embodiments, the subject has a rare muscle disease. In some embodiments, the subject has sarcopenia, muscle deterioration, decay, atrophy, cachexia, steroid myopathy, muscular dystrophy, or myopenia. In some embodiments, the subject has a fracture or other trauma. In some embodiments, the subject has a drug-induced myopathy. In some embodiments, the subject has a statin-induced myopathy. In some embodiments, the subject has a steroid-induced myopathy. In some embodiments, the subject has an immunosuppressant-induced myopathy. In some embodiments, the subject has a chemotherapeutic-induced myopathy. In some embodiments, the subject has an alcohol-induced myopathy.

In some embodiments, the subject exhibits muscle loss related to one or both of immobilization or muscle disuse following injury. In some embodiments, the subject has, or is recovering from, a surgery, e.g., rotator cuff surgery, knee surgery, or hip surgery, or has worn a cast prior to administration of the composition. In some embodiments, the subject has had, or is recovering from, a hip fracture-related myopenia prior to administration of the composition. In some embodiments, the subject has had, or is recovering from, a joint replacement prior to administration of the composition. In some embodiments, the subject has had, or is recovering from, an injury repair surgery.

In some embodiments, the subject has, or is recovering from, ventilator-induced diaphragmatic dystrophy or ventilator-induced diaphragmatic dysfunction prior to administration of the composition. In some embodiments, the subject has had one or both of ICU-acquired or burns-related myopathies.

In some embodiments, the subject has disease-related cachexia, e.g., a disease-related cachexia selected from chronic obstructive pulmonary disease (COPD), congestive heart failure (CHF), chronic kidney disease (CKD), and cancer prior to administration of the composition.

In some embodiments, the subject has perceived muscle weakness, e.g., chronic fatigue syndrome. In some embodiments, the subject has a cancer-associated muscle weakness. In some embodiments, the subject has a neuromuscular disorder, e.g., myasthenia gravis or Lambert-Eaton myasthenic syndrome. In some embodiments, the subject has muscular dystrophy, e.g., Duchenne muscular dystrophy, Becker muscular dystrophy, facioscapulohumeral muscular dystrophy, or myotonic dystrophy. In some embodiments, the subject has inflammatory myopathy, e.g., polymyositis or dermatomyositis.

In some embodiments, the subject has one, two, or more (e.g., all) of low sodium levels (e.g., hyponatremia), low potassium levels (e.g., hypokalemia), or a calcium deficiency or relatively high calcium levels (e.g., hypercalcemia).

In some embodiments, the subject has muscle weakness associated with nerve damage, e.g., neuralgia or peripheral neuropathy. In some embodiments, the subject has a bone weakness disease, e.g., osteomalacia, osteogenesis imperfecta, rickets, or Hypophosphatasia.

In some embodiments, the subject has experienced a stroke or a transient ischemic attack. In some embodiments, the subject has an autoimmune disease, e.g., Graves' disease.

In some embodiments, the subject has hypothyroidism. In some embodiments, the subject has amyotrophic lateral sclerosis (ALS).

Also provided is a method of treating one, two, three, four, five, six, seven, eight, nine, or more (e.g., all) of immobilization, malnutrition, fasting, aging, autophagy, reduced protein synthesis, anabolic resistance, junction integrity (e.g., neuromuscular junction integrity), insulin resistance, decreased mitochondrial biogenesis, an energy deficit, or anaplerosis in a subject that includes administering to a subject in need thereof an effective amount of a pharmaceutical composition including defined amino acid components. In some embodiments, the subject has a rare muscle disease. In some embodiments, the subject has sarcopenia, muscle deterioration, decay, atrophy, cachexia, drug-induced myopathy, muscular dystrophy, or myopenia. In some embodiments, the subject has a fracture or other trauma. In some embodiments, the subject has a drug-induced myopathy. In some embodiments, the subject has a statin-induced myopathy. In some embodiments, the subject has a steroid-induced myopathy. In some embodiments, the subject has an immunosuppressant-induced myopathy. In some embodiments, the subject has a chemotherapeutic-induced myopathy. In some embodiments, the subject has an alcohol-induced myopathy.

The subject may exhibit an improvement in muscle function after administration of a composition comprising a L-amino acid entity, a R-amino acid entity, a Q-amino acid entity; and an antioxidant or ROS scavenger, e.g., a NAC entity, e.g., NAC. In some embodiments, the composition further comprises one or more EAA-entities, e.g., one, two, three, or more (e.g., all) of a H-amino acid-entity, a K-amino acid-entity, a F-amino acid-entity, and a T-amino acid-entity. For example the composition may be administered to the subject for a treatment period of, e.g., two weeks, three weeks, four weeks, five weeks, six weeks, seven weeks, eight weeks, nine weeks, 10 weeks, 11 weeks, 12 weeks, 13 weeks, 14 weeks, 15 weeks, 16 weeks, or longer at a dose of, e.g., about 4 total grams per day to about 80 total grams per day (e.g., a total of about 18 g per day, 48 g per day. 68 g per day or a total of about 72 g per day).

Treatment with the composition can result in improved muscle function in a subject, e.g., by one, two, three, four, five or more (e.g., all) of activating mTORC1; improving insulin sensitivity; activating muscle protein synthesis; scavenging reactive oxygen species (ROS); decreasing inflammation (e.g., muscle inflammation); inhibiting catabolism; detoxifying ammonia; or decreasing fibrosis progression.

Improvements in muscle function can be assessed by performing metrics selected from one, two, three, four, or all of a maximal isometric knee strength test (e.g., to determine changes in muscle strength), magnetic resonance imaging (MRI, e.g., to determine total muscle volume, e.g., thigh muscle volume), muscle biopsy (e.g., to determine muscle fiber quality), a dual-energy x-ray absorptiometry (DEXA) scan (e.g., to determine body composition including lean mass and fat-free mass), and electrical impedance myography (EIM) (e.g., to determine muscle health, such as resistive and capacitive properties of muscle tissue and sensitivity to disuse-related atrophy).

In some embodiments, the composition is for use as a medicament in improving muscle function in a subject. In some embodiments, the composition is for use as a medicament in treating a muscle disease or disorder in a subject.

In some embodiments, the composition is for use in the manufacture of a medicament for improving muscle function in a subject. In some embodiments, the composition including amino acid entities is for use in the manufacture of a medicament for treating a muscle disease or disorder in a subject.

Additionally, the composition is useful as a dietary supplement.

One embodiment provides a nutritional supplement, dietary formulation, functional food, medical food, food, or beverage comprising a composition described herein. Another embodiment provides a nutritional supplement, dietary formulation, functional food, medical food, food, or beverage comprising a composition described herein for use in the management of any of the diseases or disorders described herein.

One embodiment provides a method of maintaining or improving muscle health, muscle function, muscle functional performance, or muscle strength, comprising administering to a subject an effective amount of a composition described herein. Another embodiment provides a method of providing nutritional support or supplementation to a subject suffering from muscle atrophy comprising administering to the subject an effective amount of a composition described herein. Yet another embodiment provides a method of providing nutritional support or supplementation that aids in the management of muscle atrophy to a subject comprising administering to the subject in need thereof an effective amount of a composition described herein.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, the term “amino acid entity” refers to an amino acid in one or both of free form or salt form, an amino acid residue of a peptide (e.g., of a dipeptide, oligopeptide, or polypeptide), a derivative of an amino acid, a precursor of an amino acid, or a metabolite of an amino acid.

As used herein the term “XXX amino acid entity” refers to an amino acid entity that if a free amino acid, comprises free XXX or XXX in salt form; if a peptide, refers to a peptide comprising an XXX residue; if a derivative, refers to a derivative of XXX; if a precursor, refers to a precursor of XXX; and if a metabolite, refers to a XXX metabolite. For example, where XXX is leucine (L), then L-amino acid entity refers to free L or L in salt form, a peptide comprising a L residue, a L derivative, a L precursor, or a metabolite of L; where XXX is arginine (R), then R-amino acid entity refers to free R or R in salt form, a peptide comprising a R residue, a R derivative, a R precursor, or a metabolite of R; where XXX is glutamine (Q), then Q-amino acid entity refers to free Q or Q in salt form, a peptide comprising a Q residue, a Q derivative, a Q precursor, or a metabolite of Q; where XXX is N-acetylcysteine (NAC), then NAC-amino acid entity refers to free NAC or NAC in salt form, a peptide comprising a NAC residue, a NAC derivative, a NAC precursor, or a metabolite of NAC; where XXX is histidine (H), then H-amino acid entity refers to free H or H in salt form, a peptide comprising a H residue, a H derivative, a H precursor, or a metabolite of H; where XXX is lysine (K), then K-amino acid entity refers to free K or K in salt form, a peptide comprising a K residue, a K derivative, a K precursor, or a metabolite of K; where XXX is phenylalanine (F), then F-amino acid entity refers to free F or F in salt form, a peptide comprising a F residue, a F derivative, a F precursor, or a metabolite of F; or where XXX is threonine (T), then T-amino acid entity refers to free T or T in salt form, a peptide comprising a T residue, a T derivative, a T precursor, or a metabolite of T.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

An “amino acid” refers to an organic compound having an amino group (—NH₂), a carboxylic acid group (—C(═O)OH), and a side chain bonded through a central carbon atom, and includes essential and non-amino acids, as well as natural and unnatural amino acids.

The proteogenic amino acids, shown below, are known by three- and one-letter abbreviations in addition to their full names. For a given amino acid, these abbreviations are used interchangeably herein. For example, Leu, L or leucine all refer to the amino acid leucine; Ile, I or isoleucine all refer to the amino acid isoleucine; Val, V or valine all refer to the amino acid valine; Arg, R or arginine all refer to the amino acid arginine; and Gln, Q or glutamine all refer to the amino acid glutamine Likewise, the non-natural amino acid derivative N-acetylcysteine may be referred to interchangeably by “NAC” or “N-acetylcysteine.” Amino acids may be present as D- or L-isomers. Unless otherwise indicated, amino acids referred to herein are L-isomers of amino acids.

TABLE 1
Amino acid names and abbreviations

| Amino acid | Three-letter | One-letter |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “branched chain amino acid” is an amino acid selected from leucine, isoleucine, and valine.

The term “effective amount” as used herein means an amount of an amino acid, or pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

A “pharmaceutical composition” described herein comprises at least one amino acid and a pharmaceutically acceptable carrier or excipient. In some embodiments, the pharmaceutical composition is used as a therapeutic, a nutraceutical, a medical food, or as a supplement.

The term “pharmaceutically acceptable” as used herein, refers to amino acids, materials, excipients, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A composition, formulation or product is “therapeutic” if it provides a beneficial clinical effect. A beneficial clinical effect can be shown by lessening the progression of a disease and/or alleviating one or more symptoms of the disease.

A “unit dose” or “unit dosage” as used herein means an amount or dose of medicine prepared in an individual packet or container for convenience, safety, or monitoring. A “unit dose” or “unit dosage” comprises the drug product or drug products in the form in which they are marketed for use, with a specific mixture of active ingredients and inactive components (excipients), in a particular configuration (such as a capsule shell, for example), and apportioned into a particular dose.

As used herein, the terms “treat,” “treating,” or “treatment” refer in one embodiment, to ameliorating, e.g., decreased muscle function (e.g., relative to a health subject), a muscle disease, or a muscle disorder (i.e., slowing or arresting or reducing the development of the disease or disorder or at least one of the clinical symptoms thereof). In another embodiment, “treat,” “treating,” or “treatment” refers to alleviating or ameliorating at least one physical parameter including those which may not be discernible by the patient. In yet another embodiment, “treat,” “treating,” or “treatment” refers to modulating a symptom of decreased muscle function (e.g., relative to a health subject), a muscle disease, or a muscle disorder, either physically, (e.g., stabilization of a discernible symptom), physiologically, (e.g., stabilization of a physical parameter), or both. In yet another embodiment, “treat,” “treating,” or “treatment” refers to preventing or delaying the onset or development or progression of decreased muscle function (e.g., relative to a health subject), a muscle disease, or a muscle disorder.

### Determination of Amino Acid Weight Percent and Amino Acid Ratios in a Composition

The weight ratio of a particular amino acid or particular amino acids in a composition or mixture of amino acids is the ratio of the weight of the particular amino acid or amino acids in the composition or mixture compared to the total weight of amino acids present in the composition or mixture. This value is calculated by dividing the weight of the particular amino acid or of the particular amino acids in the composition or mixture by the weight of all amino acids present in the composition or mixture.

### Compositions Comprising Amino Acid Entities

The present disclosure provides compositions, e.g., pharmaceutical compositions, comprising amino acid entities. These pharmaceutical compositions are made up of amino acid entities including amino acids in one or both of free form or salt form, amino acid residues of a peptide (e.g., of a dipeptide, oligopeptide, or polypeptide), derivatives of an amino acid, precursors of an amino acid, or metabolites of an amino acid. For example, the compositions can include a leucine (L)-amino acid entity, an arginine (R)-amino acid entity, a glutamine (Q)-amino acid entity; and an antioxidant or reactive oxygen species (ROS) scavenger, e.g., a N-acetylcysteine (NAC) entity, e.g., NAC (Table 2). In particular, at least one amino acid entity is not a peptide of more than 20 amino acid residues in length.

TABLE 2
Amino acid entities include amino acids, precursors, metabolites,
and derivatives of the compositions described herein.

| | Exemplary | | | |
|---|---|---|---|---|
| | Amino Acid | Precursors | Metabolites | Derivatives |
| L | L-Leucine | Oxo-leucine | HMB (beta- | D-Leucine; N-Acetyl- |
| | | | hydroxy-beta- | Leucine |
| | | | methybutyrate); | |
| | | | Oxo-leucine; | |
| | | | Isovaleryl-CoA | |
| I | L-Isoleucine | 2-Oxo-3-methyl- | 2-Oxo-3-methyl- | D-Isoleucine; N-Acetyl- |
| | | valerate; Threonine | valerate; | Isoleucine |
| | | | Methylbutyrl-CoA | |
| V | L-Valine | 2-Oxo-valerate | Isobutryl-CoA; 3- | D-Valine; N-Acetyl- |
| | | | HIB-CoA; 3-HIB | Valine |
| R | L-Arginine | Argininosuccinate; | Ornithine; | D-Arginine; N-Acetyl- |
| | | Citrulline; Aspartate; | Citrulline; | Arginine; |
| | | Glutamate | Agmatine; | |
| | | | Creatine | |
| Q | L-Glutamine | Glutamate | Carbamoyl-P; | D-Glutamine; N-Acetyl- |
| | | | Glutamate | Glutamine; |
| NAC | N- | Serine; Acetylserine; | Glutathione; | D-Cysteine; L-Cysteine; |
| | Acetylcysteine | Cystathionine; | Cystathionine; | Cystine; Cysteamine |
| | | | Homocysteine; | |
| | | | Methionine | |
| H | L-Histidine | Histidinol; | Carnosine; | D-Histidine; N-Acetyl- |
| | | Histidinal; | Histamine; | Histidine |
| | | Ribose-5-phosphate | Urocanate | |
| K | L-Lysine | Diaminopimelate; | Trimethyllysine; | D-Lysine; N-Acetyl- |
| | | Aspartate | Carnitine; | Lysine |
| | | | Saccharopine | |
| F | L- | Phenylpyruvate | Tyrosine | D-Phenylalanine; N- |
| | Phenylalanine | | | Acetyl-Phenylalanine |
| T | L-Threonine | Homoserine; O- | Oxobutyrate | D-Threonine; N-Acetyl- |
| | | PhosphoHomoserine | | Threonine |

In some embodiments, the total weight of the L-amino acid entity, R-amino acid entity, Q-amino acid entity; and ROS scavenger, e.g., a N-NAC entity, e.g., NAC, can be greater than the total wt. of other amino acid entities in the composition. In certain embodiments, two, three, or more (e.g., all) of methionine (M), tryptophan (W), or valine (V) may be absent from the amino acid entity composition, or if present, are present at less than 2 weight (wt.) %.

In some embodiments, one or both of the R-amino acid entity and the Q-amino acid entity are present at a higher amount (wt. %) than the L-amino acid entity. The R-amino acid entity can be present, e.g., at an amount of at least 2 wt. %, at least 3 wt. %, at least 4 wt. %, at least 5 wt. %, at least 6 wt. %, at least 7 wt. %, or at least 8 wt. % greater than the L-amino acid entity. The Q-amino acid entity can be present, e.g., at an amount of at least 2 wt. %, at least 3 wt. %, at least 4 wt. %, or at least 5 wt. % greater than the L-amino acid entity.

In some embodiments, the composition further comprises additional branched-chain amino acid (BCAA)-entities, e.g., one or both of an isoleucine (I)-amino acid-entity and a valine (V)-amino acid-entity. In some embodiments, both the I-amino acid-entity and the V-amino acid-entity are present. In certain embodiments, the L-entity is present at a higher amount (% by weight) than one or both of the I-amino acid-entity and the V-amino acid-entity (e.g., the L-entity is present at an amount of at least 10 wt. %, at least 15 wt. %, at least 20 wt. %, at least 25 wt. %, at least 30 wt. %, at least 35 wt. %, at least 40 wt. %, at least 45 wt. %, or at least 50 wt. % greater than one or both of the I-amino acid-entity and the V-amino acid-entity).

In some embodiments, the composition further comprises one or more essential amino acid (EAA)-entities. In certain embodiments the EAA-entities are chosen from one, two, three, or four of a H-amino acid-entity, a K-amino acid-entity, a F-amino acid-entity, and a T-amino acid-entity.

In an embodiment, the H-amino acid-entity is present. In certain embodiments, the H-amino acid-entity is present in an amount of at least 0.5 wt. %, at least 0.6 wt. %, at least 0.7 wt. %, at least 0.8 wt. %, at least 0.9 wt. %, at least 1.0 wt. %, at least 1.1 wt. %, at least 1.2 wt. %, at least 1.3 wt. % or at least 1.4 wt. % of the composition.

In an embodiment, the K-amino acid-entity is present. In certain embodiments, the K-amino acid-entity is present in amount of at least 2 wt. %, at least 3 wt. %, at least 4 wt. %, at least 5 wt. %, or at least 6 wt. % of the composition.

In an embodiment, the F-amino acid-entity is present. In certain embodiments, the F-amino acid-entity is present in an amount of at least 0.5 wt. %, at least 0.6 wt. %, at least 0.7 wt. %, at least 0.8 wt. %, at least 0.9 wt. %, at least 1.0 wt. %, at least 1.1 wt. %, at least 1.2 wt. %, at least 1.3 wt. % or at least 1.4 wt. % of the composition.

In an embodiment, the T-amino acid-entity is present. In certain embodiments, the T-amino acid-entity is present in amount of at least 0.5 wt. %, at least 1 wt. %, at least 1.5 wt. %, at least 2 wt. %, at least 2.5%, or at least 3 wt. % of the composition.

In certain embodiments, H-amino acid entity, K-amino acid entity, F-amino acid entity, and T-amino acid entity are present in the composition.

In some embodiments, the L-amino acid entity is selected from the group consisting of a precursor, a metabolite, and a derivative. In certain embodiments, the L-amino acid entity is selected from the group consisting of L-leucine, β-hydroxy-β-methylbutyrate (HMB), oxo-leucine, isovaleryl-CoA, D-leucine, and n-acetylleucine. In one embodiment, the L-amino acid entity is L-leucine. In another embodiment, the L-amino acid entity is HMB.

In some embodiments, the R-amino acid entity is selected from the group consisting of a precursor, a metabolite, and a derivative. In certain embodiments, the R-amino acid entity is selected from the group consisting of L-arginine, D-arginine, ornithine, argininosuccinate, citrulline, aspartate, glutamate, agmatine, and N-acetyl-arginine. In one embodiment, the R-amino acid entity is L-arginine. In one embodiment, the R-amino acid entity is creatine. In another embodiment, the R-amino acid entity is ornithine.

In some embodiments, the Q-amino acid entity is selected from the group consisting of a precursor, a metabolite, and a derivative. In certain embodiments, the Q-amino acid entity is selected from the group consisting of L-glutamine, glutamate, carbamoyl-P, glutamate, D-glutamine, and n-acetylglutamine. In one embodiment, the Q-amino acid entity is L-glutamine.

In some embodiments, the NAC-amino acid entity is selected from the group consisting of a precursor, a metabolite, and a derivative. In certain embodiments, the NAC-amino acid entity is selected from the group consisting NAC, serine, acetylserine, cystathionine, cystathionine, homocysteine, methionine, glutathione, D-cysteine, and L-cysteine. In one embodiment, the NAC entity is NAC. In one embodiment, the NAC entity is glutathione.

In some embodiments, the I-amino acid entity is selected from the group consisting of a salt, a precursor, a metabolite, and a derivative. In certain embodiments, the I-amino acid entity is selected from the group consisting of L-isoleucine, 2-oxo-3-methyl-valerate, threonine, 2-oxo-3-methyl-valerate, methylbutyrl-CoA, D-isoleucine, and N-acetyl-isoleucine. In one embodiment, the I-amino acid entity is L-isoleucine.

In some embodiments, the V-amino acid entity is selected from the group consisting of a precursor, a metabolite, and a derivative. In certain embodiments, the V-amino acid entity is selected from the group consisting of L-valine, 2-oxo-valerate, isobutryl-CoA, 3-HIB-CoA, 3-HIB, D-valine, and N-acetyl-valine. In one embodiment, the I-amino acid entity is L-valine.

In some embodiments, the H-amino acid entity is selected from the group consisting of a precursor, a metabolite, and a derivative. In certain embodiments, the H-amino acid entity is selected from the group consisting of L-histidine, histidinol, histidinal, ribose-5-phosphate, carnosine, histamine, urocanate, D-histidine, and N-acetyl-histidine. In certain embodiments, the H-amino acid entity is an amino acid, e.g., L-histidine. In certain embodiments, the H-amino acid entity is a precursor, e.g., histidinol, histidinal, or ribose-5-phosphate. In certain embodiments, the H-amino acid entity is a metabolite, e.g., carnosine, histamine, or urocanate. In certain embodiments, the H-amino acid entity is a derivative, e.g., D-histidine or N-acetyl-histidine.

In some embodiments, the K-amino acid entity is selected from the group consisting of a precursor, a metabolite, and a derivative. In certain embodiments, the K-amino acid entity is selected from the group consisting of L-lysine, diaminopimelate, aspartate, trimethyllysine, carnitine, saccharopine, D-lysine, and N-acetyl-lysine. In certain embodiments, the K-amino acid entity is an amino acid, e.g., L-lysine. In certain embodiments, the K-amino acid entity is a precursor, e.g., diaminopimelate or aspartate. In certain embodiments, the K-amino acid entity is a metabolite, e.g., trimethyllysine, carnitine, or saccharopine. In certain embodiments, the K-amino acid entity is a derivative, e.g., D-lysine or N-acetyl-lysine.

In some embodiments, the F-amino acid entity is selected from the group consisting of a precursor, a metabolite, and a derivative. In certain embodiments, the F-amino acid entity is selected from the group consisting of L-phenylalanine, phenylpyruvate, tyrosine, D-phenylalanine, and N-acetyl-phenylalanine. In certain embodiments, the F-amino acid entity is an amino acid, e.g., L-phenylalanine. In certain embodiments, the F-amino acid entity is a precursor, e.g., phenylpyruvate. In certain embodiments, the F-amino acid entity is a metabolite, e.g., tyrosine. In certain embodiments, the F-amino acid entity is a derivative, e.g., D-phenylalanine, and N-acetyl-phenylalanine.

In some embodiments, the T-amino acid entity is selected from the group consisting of a precursor, a metabolite, and a derivative. In certain embodiments, the T-amino acid entity is selected from the group consisting of L-threonine, homoserine, O-phosphohomoserine, oxobutyrate, D-threonine, and N-acetyl-threonine. In certain embodiments, the T-amino acid entity is an amino acid, e.g., L-threonine. In certain embodiments, the T-amino acid entity is a precursor, e.g., homoserine or O-phosphohomoserine. In certain embodiments, the T-amino acid entity is a metabolite, e.g., oxobutyrate. In certain embodiments, the T-amino acid entity is a derivative, e.g., D-threonine or N-acetyl-threonine.

In some embodiments, the derivative of an amino acid entity comprises an amino acid ester (e.g., an alkyl ester, e.g., an ethyl ester or a methyl ester of an amino acid entity) or a keto-acid.

In some embodiments, the composition comprises L-leucine or a leucine metabolite (e.g., HMB), L-arginine or an L-arginine metabolite (e.g., creatine or ornithine), L-glutamine, and NAC or a NAC metabolite, e.g., glutathione. In one embodiment, the composition comprises L-leucine, L-arginine, L-glutamine, and NAC. In one embodiment, the composition comprises HMB, creatine, L-glutamine, and glutathione. In one embodiment, the composition comprises HMB, ornithine, L-glutamine, and glutathione. In one embodiment, the composition comprises HMB, L-arginine, L-glutamine, and NAC. In one embodiment, the composition comprises L-leucine, creatine, L-glutamine, and NAC. In one embodiment, the composition comprises L-leucine, ornithine, L-glutamine, and NAC. In one embodiment, the composition comprises L-leucine, L-arginine, L-glutamine, and glutathione. In some embodiments, the composition further comprises one or more EAA-entities. In certain embodiments the EAA-entities are chosen from one, two, three, or four of a H-amino acid-entity, a K-amino acid-entity, a F-amino acid-entity, and a T-amino acid-entity.

In some embodiments, the NAC entity is more stable than cysteine. In certain embodiments, the NAC entity does not comprise cysteine. In some embodiments, the NAC entity promotes the formation of glutathione (GSH).

In some embodiments, the weight (wt.) ratio of the L-amino acid entity, the R-amino acid entity, the Q-amino acid entity, and the NAC-amino acid entity is about 1-3:2-4:2-4:0.1-2.5. In certain embodiments, the wt. ratio of the L-amino acid entity, the R-amino acid entity, the Q-amino acid entity, and the NAC-amino acid entity is about 2:3:2.66:0.3. In certain embodiments, the wt. ratio of the L-amino acid entity, the R-amino acid entity, the Q-amino acid entity, and the NAC-amino acid entity is about 2:3:2.66:0.6.

In some embodiments, the composition comprises a ratio of branched-chain amino acids to total amino acids of about 4:7 to about 1:2.

In some embodiments, the wt. ratio of the L-amino acid entity, the I-amino acid entity, the V-amino acid entity, the R-amino acid entity, the Q-amino acid entity, the NAC-amino acid entity, the H-amino acid entity, the K-amino acid entity, the F-amino acid entity, and the T-amino acid entity is about 1-3:0.5-1.5:0.5-1.5:2-4:2-4:0.1-0.5:0.1-0.5:0.2-1.0:0.1-0.5:0.2-0.7. In certain embodiments, the wt. ratio of the L-amino acid entity, the I-amino acid entity, the V-amino acid entity, the R-amino acid entity, the Q-amino acid entity, the NAC-amino acid entity, the H-amino acid entity, the K-amino acid entity, the F-amino acid entity, and the T-amino acid entity is about 2.0:1.0:1.0:3.0:2.66:0.3:0.16:0.7:0.16:0.34. In certain embodiments, the wt. ratio of the L-amino acid entity, the I-amino acid entity, the V-amino acid entity, the R-amino acid entity, the Q-amino acid entity, the NAC-amino acid entity, the H-amino acid entity, the K-amino acid entity, the F-amino acid entity, and the T-amino acid entity is about 2.0:1.0:1.0:3.0:2.66:0.3:0.16:0.7:0.16:0.68.

In some embodiments, the total wt. of amino acids present is between about 4 g and about 80 g. In certain embodiments, the total wt. of amino acids present is about 6 g, about 18 g, about 24 g, about 48 g, about 68 g, or about 72 g.

In some embodiments, the composition comprises at least 1 g of the L-amino acid entity, at least 0.5 g of the I-amino acid entity, at least 0.5 g of the V-amino acid entity, at least 1.5 g of the R-amino acid entity, at least 1.33 g of the Q-amino acid entity, at least 0.15 g of the NAC-amino acid entity, at least 0.08 g of the H-amino acid entity, at least 0.35 g of the K-amino acid entity, at least 0.08 g of the F-amino acid entity, and at least 0.17 g of the T-amino acid entity. In some embodiments, the composition comprises at least 1 g of the L-amino acid entity, at least 0.5 g of the I-amino acid entity, at least 0.5 g of the V-amino acid entity, at least 1.5 g of the R-amino acid entity, at least 1.33 g of the Q-amino acid entity, at least 0.3 g of the NAC-amino acid entity, at least 0.08 g of the H-amino acid entity, at least 0.35 g of the K-amino acid entity, at least 0.08 g of the F-amino acid entity, and at least 0.17 g of the T-amino acid entity.

In some embodiments, the composition comprises at least 3 g of the L-amino acid entity, at least 1.5 g of the I-amino acid entity, at least 1.5 g of the V-amino acid entity, at least 4.5 g of the R-amino acid entity, at least 3.99 g of the Q-amino acid entity, at least 0.45 g of the NAC-amino acid entity, at least 0.24 g of the H-amino acid entity, at least 1.05 g of the K-amino acid entity, at least 0.24 g of the F-amino acid entity, and at least 0.51 g of the T-amino acid entity. In some embodiments, the composition comprises at least 3 g of the L-amino acid entity, at least 1.5 g of the I-amino acid entity, at least 1.5 g of the V-amino acid entity, at least 4.5 g of the R-amino acid entity, at least 3.99 g of the Q-amino acid entity, at least 0.9 g of the NAC-amino acid entity, at least 0.24 g of the H-amino acid entity, at least 1.05 g of the K-amino acid entity, at least 0.24 g of the F-amino acid entity, and at least 0.51 g of the T-amino acid entity.

In some embodiments, the amino acids comprise about 10 wt % to about 20 wt % the L-amino acid entity, about 5 wt % to about 15 wt % the I-amino acid entity, about 5 wt % to about 15 wt % the V-amino acid entity, about 20 wt % to about 40 wt % the R-amino acid entity, about 15 wt % to about 35 wt % the Q-amino acid entity, about 1 wt % to about 10 wt % the NAC-amino acid entity, about 0.5 wt % to about 5 wt % the H-amino acid entity, about 3 wt % to about 8 wt % the K-amino acid entity, about 0.5 wt % to about 5 wt % phenylalanine, and about 1 wt % to about 8 wt % threonine.

In some embodiments, at least one amino acid entity is a free amino acid, e.g., one, two, three, four, five, six, seven, eight, nine, or more (e.g., all) amino acid entities are a free amino acid. In some embodiments, the L-amino acid entity, the R-amino acid entity, the Q-amino acid entity, and the NAC-amino acid entity is a free amino acid entity. In certain embodiment, the L-amino acid entity, the I-amino acid entity, the V-amino acid entity, the R-amino acid entity, the Q-amino acid entity, and the NAC-amino acid entity a free amino acid. In certain embodiments, the L-amino acid entity, the I-amino acid entity, the V-amino acid entity, the R-amino acid entity, the Q-amino acid entity, the NAC-amino acid entity, the H-amino acid entity, the K-amino acid entity, the F-amino acid entity, and the T-amino acid entity is a free amino acid.

In some embodiments, at least one amino acid entity is in a salt form, e.g., one, two, three, four, five, six, seven, eight, nine, or more (e.g., all) of the amino acid entities is in a salt form. In some embodiments, wherein the L-amino acid entity, the R-amino acid entity, the Q-amino acid entity, and the NAC-amino acid entity is in a salt form. In certain embodiments, the L-amino acid entity, the I-amino acid entity, the V-amino acid entity, the R-amino acid entity, the Q-amino acid entity, and the NAC-amino acid entity is in a salt form. In certain embodiments, the L-amino acid entity, the I-amino acid entity, the V-amino acid entity, the R-amino acid entity, the Q-amino acid entity, the NAC-amino acid entity, the H-amino acid entity, the K-amino acid entity, the F-amino acid entity, and the T-amino acid entity is in a salt form.

In some embodiments, the composition comprises a combination of 2 to 20 different amino acid entities, e.g., 5 to 15 different amino acid entities.

In some embodiments, the composition further comprises one, two, three, four, five, six, seven, eight, nine, ten, or more (e.g., all) or more of serine, glycine, glutamine, HMB, arginine, L-leucine, citrulline, glutamine, ornithine, L-cysteine, cystine, or glutathione.

In some embodiments, the composition further comprises EAA-entities (e.g., EAA-entities chosen from one, two, three, or four of a H-amino acid-entity, a K-amino acid-entity, a F-amino acid-entity, and a T-amino acid-entity) and a protein source of EAAs.

In other embodiments, the composition further comprises a protein source of EAAs instead of EAA-entities (e.g., EAA-entities chosen from one, two, three, or four of a H-amino acid-entity, a K-amino acid-entity, a F-amino acid-entity, and a T-amino acid-entity). In some embodiments, the composition comprises leucine, isoleucine, valine, arginine, glutamine, N-acetylcysteine, histidine, lysine, phenylalanine, and threonine.

In some embodiments, the composition comprises arginine, glutamine, N-acetylcysteine; a BCAA chosen from one, two, or all of leucine, isoleucine, and valine; and an essential amino acid EAA chosen from one, two, or all of histidine, lysine, and threonine.

In some embodiments, the BCAA is leucine.

In some embodiments, the BCAA is isoleucine.

In some embodiments, the BCAA is valine.

In some embodiments, the BCAA is leucine and isoleucine.

In some embodiments, the BCAA is leucine and valine.

In some embodiments, the BCAA is isoleucine and valine.

In some embodiments, the BCAA is leucine, isoleucine, and valine.

In some embodiments, the EAA is histidine.

In some embodiments, the EAA is lysine.

In some embodiments, the EAA is threonine.

In some embodiments, the EAA is histidine and lysine.

In some embodiments, the EAA is lysine and threonine.

In some embodiments, the EAA is histidine, lysine, and threonine.

An aspect of the present disclosure provides a composition comprising free amino acids and one or more pharmaceutically acceptable excipients, such that the amino acids include leucine, isoleucine, valine, arginine, glutamine, N-acetylcysteine, histidine, lysine, phenylalanine, and threonine.

An aspect of the present disclosure provides a composition comprising free amino acids and one or more pharmaceutically acceptable excipients, such that the amino acids consist of leucine, isoleucine, valine, arginine, glutamine, N-acetylcysteine, histidine, lysine, phenylalanine, and threonine.

In some embodiments, the composition includes a ratio of branched-chain amino acids to total amino acids of about 4:7 to about 1:2. In an embodiment, the composition includes a ratio of branched-chain amino acids to total amino acids of about 4:7. In an embodiment, the composition includes a ratio of branched-chain amino acids to total amino acids of about 1:2.

In some embodiments, leucine, isoleucine, valine, arginine, glutamine, N-acetylcysteine, histidine, lysine, phenylalanine, and threonine are present in a weight ratio of about 2.0:1.0:1.0:3.0:2.66:0.3:0.16:0.7:0.16:0.34.

In some embodiments the arginine comprises arginine HCl. In some embodiments, leucine, isoleucine, valine, arginine HCl, glutamine, N-acetylcysteine, histidine, lysine, phenylalanine, and threonine are present in a weight ratio of about 2.0:1.0:1.0:3.62:2.66:0.3:0.16:0.7:0.16:0.34.

In some embodiments, leucine, isoleucine, valine, arginine, glutamine, N-acetylcysteine, histidine, lysine, phenylalanine, and threonine are present in a weight ratio of about 2:1:1:3:4:0.5:0.16:0.5:0.16:0.34.

In some embodiments, the amino acids leucine, isoleucine, valine, arginine, glutamine, N-acetylcysteine, histidine, lysine, phenylalanine, and threonine are present in a weight ratio of about 2:1:1:3:2.67:0.3:0.17:0.5:0.17:0.34.

In some embodiments, the total weight of amino acids present is between about 4 g and about 80 g. In some embodiments, the total weight of amino acids present is between about 4 g and about 15 g (e.g., about 6 g). In some embodiments, the total weight of amino acids present is between about 15 g and about 20 g (e.g., about 18 g). In some embodiments, the total weight of amino acids present is between about 20 g and about 40 g (e.g., about 24 g). In some embodiments, the total weight of amino acids present is between about 40 g and about 80 g (e.g., about 72 g).

In some embodiments, the composition includes at least 1 g of leucine, at least 0.5 g of isoleucine, at least 0.5 g of valine, at least 1.5 g of arginine, at least 1.33 g of glutamine, at least 0.15 g of N-acetylcysteine, at least 0.08 g of histidine, at least 0.35 g of lysine, at least 0.08 g of phenylalanine, and at least 0.17 g of threonine.

In some embodiments, the composition includes about 1 g of leucine, about 0.5 g of isoleucine, about 0.5 g of valine, about 1.5 g of arginine, about 1.33 g of glutamine, about 0.15 g of N-acetylcysteine, about 0.08 g of histidine, about 0.35 g of lysine, about 0.08 g of phenylalanine, and about 0.17 g of threonine.

In some embodiments, the composition includes at least 3 g of leucine, at least 1.5 g of isoleucine, at least 1.5 g of valine, at least 4.5 g of arginine, at least 3.99 g of glutamine, at least 0.45 g of N-acetylcysteine, at least 0.24 g of histidine, at least 1.05 g of lysine, at least 0.24 g of phenylalanine and at least 0.51 g of threonine. In an embodiment, the composition includes about 3 g of leucine, about 1.5 g of isoleucine, about 1.5 g of valine, about 4.5 g of arginine, about 3.99 g of glutamine, about 0.45 g of N-acetylcysteine, about 0.24 g of histidine, about 1.05 g of lysine, about 0.24 g of phenylalanine, and about 0.51 g of threonine.

In some embodiments, the composition includes about 4.0 g of leucine, about 2.0 g of isoleucine, about 2.0 g of valine, about 6.0 g of arginine (or about 7.2 g of arginine HCl), about 5.33 g of glutamine, about 0.6 g of N-acetylcysteine, about 0.32 g of histidine, about 1.4 g of lysine, about 0.32 g of phenylalanine and about 0.68 g of threonine.

In some embodiments, the amino acids include about 10 wt % to about 20 wt % leucine, about 5 wt % to about 15 wt % isoleucine, about 5 wt % to about 15 wt % valine, about 20 wt % to about 40 wt % arginine, about 15 wt % to about 35 wt % glutamine, about 1 wt % to about 10 wt % N-acetylcysteine, about 0.5 wt % to about 5 wt % histidine, about 3 wt % to about 8 wt % lysine, about 0.5 wt % to about 5 wt % phenylalanine, and about 1 wt % to about 8 wt % threonine.

An exemplary Amino Acid Composition includes leucine, isoleucine, valine, arginine HCl, glutamine, N-acetylcysteine, histidine, lysine, phenylalanine, and threonine as its defined amino acid components in a wt. ratio of 2.0:1.0:1.0:3.62:2.66:0.3:0.16:0.7:0.16:0.34 (Table 3). The Amino Acid Composition includes leucine, isoleucine, valine, arginine, glutamine, N-acetylcysteine, histidine, lysine, phenylalanine, and threonine as its defined amino acid components in a wt. ratio of 2.0:1.0:1.0:3.0:2.66:0.3:0.16:0.7:0.16:0.34.

TABLE 3
Exemplary amino acid components of the composition.

| | | | | Total g | | Total g |
|---|---|---|---|---|---|---|
| | weight | | | daily | | daily |
| Amino acid | ratio | g/packet | g/dose 1 | dose 1 | g/dose 2 | dose 2 |
| Leucine | 2.0 | 1.0 | 1.0 | 3 | 4 | 12 |
| Isoleucine | 1.0 | 0.5 | 0.5 | 1.5 | 2 | 6 |
| Valine | 1.0 | 0.5 | 0.5 | 1.5 | 2 | 6 |
| Arginine HCl | 3.62 | 1.81 | 1.81 | 5.43 | 7.24 | 21.72 |
| Glutamine | 2.66 | 1.33 | 1.33 | 3.99 | 5.32 | 15.96 |
| N-acetyl- | 0.3 | 0.15 | 0.15 | 0.45 | 0.6 | 1.8 |
| cysteine | | | | | | |
| Histidine | 0.16 | 0.08 | 0.08 | 0.24 | 0.32 | 0.96 |
| Lysine | 0.7 | 0.35 | 0.35 | 1.05 | 1.4 | 4.2 |
| Phenylalanine | 0.16 | 0.08 | 0.08 | 0.24 | 0.32 | 0.96 |
| Threonine | 0.34 | 0.17 | 0.17 | 0.51 | 0.68 | 2.04 |
| Total | | ~6 g | ~6 g | ~18 g | ~24 g | ~72 g |
| amino acids | | | | | | |

The composition is administered in packets including about 6 g total amino acids.

In some embodiments, the composition is administered three times daily at a dose of about 6 g total amino acids. In some embodiments, about 18 g, about 22, about 24 g, about 68 g or about 72 g total amino acids is administered per day to, e.g., enhance muscle function in a subject (e.g., the subject has or is identified as having decreased muscle function due to aging, injury, atrophy, infection, or disease). In some embodiments, about 18 g, about 22, about 24 g, about 68 g, or about 72 g total amino acids is administered per day to, e.g., treat one, two, three, four, five, six, seven, eight, nine, or more (e.g., all) of immobilization, malnutrition, fasting, aging, autophagy, reduced protein synthesis, anabolic resistance, junction integrity (e.g., neuromuscular junction integrity), insulin resistance, decreased mitochondrial biogenesis, anaplerosis, or an energy deficit in a subject in need thereof.

In some embodiments, the composition is administered three times daily at a dose of about 24 g total amino acids. In some embodiments, about 48 g total amino acids administered per day. In some embodiments, about 68 g total amino acids iadministered per day. In some embodiments, about 72 g total amino acids is administered per day to enhance muscle function in a subject (e.g., the subject has or is identified as having decreased muscle function due to aging, injury, atrophy, infection, or disease). In some embodiments, about 68 or about 72 g total amino acids is administered per day to, e.g., treat one, two, three, four, five, six, seven, eight, nine, or more (e.g., all) of immobilization, malnutrition, fasting, aging, autophagy, reduced protein synthesis, anabolic resistance, junction integrity (e.g., neuromuscular junction integrity, insulin resistance, decreased mitochondrial biogenesis, anaplerosis, or an energy deficit in a subject in need thereof.

The disclosure also provides a composition comprising at least four different amino acid entities, in which the composition is capable of one, two, three, or all of:
a) activating mTORC1;
b) activating protein synthesis and/or inhibiting protein catabolism;
c) improving, e.g., increasing, insulin sensitivity or glucose tolerance; or
d) reducing inflammation;

provided that at least one amino acid entity is not a polypeptide of more than 20 amino acid residues in length.

The disclosure also provides a composition comprising at least four different amino acid entities, wherein said composition when administered to a subject results in one, two, three, or all of:
a) activating mTORC1;
b) activating protein synthesis and/or inhibiting protein catabolism;
c) improving insulin sensitivity or glucose tolerance; or
d) reducing inflammation;

provided that at least one amino acid entity is not a polypeptide of more than 20 amino acid residues in length.

In some embodiments, the protein synthesis is muscle protein synthesis. In some embodiments, the protein catabolism is muscle protein catabolism

In some embodiments, the composition that activates mTORC1 comprises one or more branched-chain amino acid (BCAAs), one or more conditionally essential amino acids (CEAAs), one or more essential amino acid (EAAs), and an antioxidant or reactive oxygen species (ROS) scavenger.

In some embodiments, the at least one amino acid entity that activates protein synthesis or inhibits protein catabolism comprises one or more BCAAs, one or more CEAAs, one or more EAAs, and an antioxidant or ROS scavenger.

In some embodiments, the at least one amino acid entity that improves insulin sensitivity or glucose tolerance comprises one or more BCAAs, one or more CEAAs, one or more EAAs, and an antioxidant or ROS scavenger.

In some embodiments, the at least one amino acid entity that reduces inflammation comprises one or more BCAAs, one or more CEAAs, one or more EAAs, and an antioxidant or ROS scavenger.

In some embodiments, the BCAA comprises a L-amino acid entity.

In some embodiments, the BCAAs comprise a L-amino acid entity and an I-amino acid entity.

In some embodiments, the BCAAs comprise a L-amino acid entity and a V-amino acid entity.

In some embodiments, the BCAAs comprise a L-amino acid entity, a V-amino acid entity, and an I-amino acid entity.

In some embodiments, the CEAA comprises a R-amino acid entity.

In some embodiments, the CEAA comprises a Q-amino acid entity.

In some embodiments, the CEAAs comprises a R-amino acid entity and a Q-amino acid entity.

In some embodiments, the antioxidant or ROS scavenger comprises a NAC entity, e.g., NAC.

In some embodiments, the EAA-entities are chosen from one, two, three, or four of a H-amino acid-entity, a K-amino acid-entity, a F-amino acid-entity, and a T-amino acid-entity.

In some embodiments, the composition is capable of activating mTORC1 by at least 20%, 25%, 30%, 35%, 40%, 45%, 50%, 55%, 60%, 65%, 70%, 75%, 80%, 85%, 90%, 95%, or 99%, as detected using as an assay to measure mTORC1 substrate phosphorylation, e.g., P-rpS6 phosphorylation, e.g., an ELISA and/or cellular kinase assay, e.g., as described in Example 1, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-arginine, L-glutamine, and NAC; L-glutamine; or NAC).

In some embodiments, the composition is capable of phosphorylating an mTORC1 substrate e.g., P-rpS6 phosphorylation by at least 20%, 25%, 30%, 35%, 40%, 45%, 50%, 55%, 60%, 65%, 70%, 75%, 80%, 85%, 90%, 95%, or 99%, as detected using as assay to measure mTORC1 substrate phosphorylation, e.g., P-rpS6 phosphorylation, e.g., an ELISA and/or cellular kinase assay, e.g., as described in Example 1, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-arginine, L-glutamine, and NAC; L-glutamine; or NAC).

In some embodiments, the composition is capable of increasing myogenesis by at least 20%, 25%, 30%, 35%, 40%, 45%, 50%, 55%, 60%, 65%, 70%, 75%, 80%, 85%, 90%, 95%, or 99%, as detecting by counting myoblasts cells, e.g., C2C12 cells, e.g., by a nuclear stain, e.g., a Hoechst stain, e.g., as described in Example 2, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-leucine, L-isoleucine, L-valine, L-arginine, and NAC; L-glutamine, and NAC; L-glutamine; NAC; or an amino acid composition comprising L-leucine, L-arginine, L-glutamine, NAC, L-histidine, L-lysine, L-phenylanine, and L-threonine).

In some embodiments, the composition is capable of increasing myoblast cell count by at least 20%, 25%, 30%, 35%, 40%, 45%, 50%, 55%, 60%, 65%, 70%, 75%, 80%, 85%, 90%, 95%, or 99%, as detecting by counting myoblasts cells, e.g., C2C12 cells, e.g., by a nuclear stain, e.g., a Hoechst stain, e.g., as described in Example 2, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-leucine, L-isoleucine, L-valine, L-arginine, and NAC; L-glutamine, and NAC; L-glutamine; NAC; or an amino acid composition comprising L-leucine, L-arginine, L-glutamine, NAC, L-histidine, L-lysine, L-phenylanine, and L-threonine).

In some embodiments, the composition is capable of increasing myotube growth by at least 20%, 25%, 30%, 35%, 40%, 45%, 50%, 55%, 60%, 65%, 70%, 75%, 80%, 85%, 90%, 95%, or 99%, by detecting an increase of MyoD and/or Myogenin in, e.g., C2C12 cells, e.g., as detected using as immunohistochemistry, e.g., as described in Example 3, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-leucine, L-isoleucine, L-valine, L-arginine, and NAC; L-glutamine, and NAC; L-glutamine; NAC; or an amino acid composition comprising L-leucine, L-arginine, L-glutamine, NAC, L-histidine, L-lysine, L-phenylanine, and L-threonine).

In some embodiments, the composition is capable of increasing MyoD and/or Myogenin by at least 20%, 25%, 30%, 35%, 40%, 45%, 50%, 55%, 60%, 65%, 70%, 75%, 80%, 85%, 90%, 95%, or 99%, as detecting by detecting an increase of MyoD and/or Myogenin in, e.g., C2C12 cells, e.g., as detected using as immunohistochemistry, e.g., as described in Example 3, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-leucine, L-isoleucine, L-valine, L-arginine, and NAC; L-glutamine, and NAC; L-glutamine; NAC; or an amino acid composition comprising L-leucine, L-arginine, L-glutamine, NAC, L-histidine, L-lysine, L-phenylanine, and L-threonine).

In some embodiments, the composition is capable of activating protein synthesis and/or inhibiting protein catabolism by at least 25%, 30%, 35%, 40%, 45%, 50%, 55%, 60%, 65%, 70%, 75%, 80%, 85%, 90%, 95%, or 99%, as detected using an assay to measure Fractional Synthetic Rates (FSR) either in cultured myotubes or rodents, e.g., relative to a reference composition.

In some embodiments, the composition is capable of inhibiting protein catabolism by at least 25%, 30%, 35%, 40%, 45%, 50%, 55%, 60%, 65%, 70%, 75%, 80%, 85%, 90%, 95%, or 99%, as detected using an assay to measure proteasomal activity, e.g., proteasomal activity in muscle tissue, e.g., proteasomal activity in skeletal muscle tissue, e.g., relative to a reference composition.

In some embodiments, the composition is capable of improving insulin sensitivity or glucose tolerance by at least 25%, 30%, 35%, 40%, 45%, 50%, 55%, 60%, 65%, 70%, 75%, 80%, 85%, 90%, 95%, or 99%, as detected using as assay to measure insulin-stimulated glucose disposal or glucose-induced insulin secretion either in cultured myotubes or rodents, e.g., relative to a reference composition.

In some embodiments, the composition is capable of reducing inflammation by at least 25%, 30%, 35%, 40%, 45%, 50%, 55%, 60%, 65%, 70%, 75%, 80%, 85%, 90%, 95%, or 99%, as detected using as assay to measure cytokine or collagen production either in cells or in vivo, e.g., relative to a reference composition.

In some embodiments, the reference composition comprises a single amino acid entity, e.g., a L-amino acid entity, an I-amino acid entity, a V-amino acid entity, a R-amino acid entity, a Q-amino acid entity, or a NAC-amino acid entity, each assayed separately as a free amino acid, or a combination of amino acid entities (e.g., a L-amino acid entity, an I-amino acid entity, and a V-amino acid entity; a R-amino acid entity, a Q-amino acid entity, and a NAC-amino acid entity; a L-amino acid entity, an I-amino acid entity, V-amino acid entity, a R-amino acid entity, and a Q-amino acid entity). In certain embodiments, the reference composition comprises vehicle (e.g., PBS or saline).

### Production of the Amino Acid Compositions

Amino acids used to make the compositions may be agglomerated, and/or instantized to aid in dispersal and/or solubilization.

The amino acid compositions of the present disclosure may be made using amino acids and amino acid derivatives from the following sources, or other sources may used: e.g., FUSI-BCAA™ Instantized Blend (L-Leucine, L-Isoleucine and L-Valine in 2:1:1 weight ratio), FUSIL™ Instantized L-Leucine, L-Arginine HCl, L-Glutamine and other amino acids may be obtained from Ajinomoto Co., Inc; N-acetyl-cysteine may be obtained from Spectrum Chemical.

To produce the amino acid compositions of the instant disclosure, the following general steps may be used: the starting materials (individual amino acids and excipients) may be blended in a blending unit, followed by verification of blend uniformity and amino acid content, and filling of the blended powder into stick packs or other unit dosage form. The content of stick packs or other unit dosage forms may be dispersed in water at time of use for oral administration.

### Formulations

The pharmaceutical compositions of the present disclosure may be in a form suitable for oral use (for example as tablets, lozenges, hard or soft capsules, aqueous or oily suspensions, emulsions, dispersible powders or granules, syrups or elixirs, medical food products, nutraceuticals), for topical use (for example as creams, ointments, gels, or aqueous or oily solutions or suspensions), for administration by inhalation (for example as finely divided powder) for parental administration (for example as a sterile aqueous or oily solution for intravenous, subcutaneous, intramuscular dosing or as a suppository for rectal dosing) or for enteral administration (for example via tube feeding).

### Excipients

The amino acid compositions of the present disclosure may be compounded or formulated with one or more excipients. Non-limiting examples of suitable excipients include a tastant, a flavorant, a buffering agent, a preservative, a stabilizer, a binder, a compaction agent, a lubricant, a dispersion enhancer, a disintegration agent, a flavoring agent, a sweetener, and a coloring agent.

In some embodiments, the excipient comprises a buffering agent. Non-limiting examples of suitable buffering agents include citric acid, sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, xanthan gum, bentonite, purified wood cellulose, sodium starch glycolate, isoamorphous silicate, and microcrystalline cellulose as high HLB emulsifier surfactants.

In some embodiments, the composition comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, the composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

Particular excipients may include one or more of: citric acid, lecithin, (e.g. Alcolec F100), sweeteners (e.g. sucralose, sucralose micronized NF, acesulfame potassium (e.g. Ace-K)), a dispersion enhancer (e.g. xanthan gum (e.g. Ticaxan Rapid-3)), flavorings (e.g. vanilla custard #4306, Nat Orange WONF #1326, lime 865.0032U, and lemon 862.2169U), a bitterness masking agent (e.g. 936.2160U), and natural or artificial colorings (e.g. FD&C Yellow 6).

### Methods of Treatment

The composition as described herein can be administered to improve, e.g., enhance, muscle function, e.g., in a patient with a muscle disease or disorder. The present disclosure also provides a method for treating one, two, three, four, five, six, seven, eight, nine, or more (e.g., all) physiological symptoms selected from immobilization, malnutrition, fasting, aging, autophagy, reduced protein synthesis, anabolic resistance, neuromuscular junction integrity, insulin resistance, decreased mitochondrial biogenesis, anaplerosis, or an energy deficit. The method includes administering to a subject in need thereof an effective amount of the composition. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the subject has a muscle disease or disorder. In some embodiments, the muscle disease or disorder is a dystrophy. In some embodiments, the muscle disease or disorder is a myotonic dystrophy. In some embodiments, the muscle disease or disorder is DM1.

In some embodiments, the muscle disease or disorder is a drug-induced myopathy. In some embodiments, the muscle disease or disorder is a statin-induced myopathy. In some embodiments, the muscle disease or disorder is a steroid-induced myopathy. In some embodiments, the muscle disease or disorder is an immunosuppressant-induced myopathy. In some embodiments, the muscle disease or disorder is a chemotherapeutic-induced myopathy. In some embodiments, the muscle disease or disorder is an alcohol-induced myopathy.

In some embodiments, the subject has a fracture or other trauma. In some embodiments, the subject has a drug-induced myopathy. In some embodiments, the subject has a statin-induced myopathy. In some embodiments, the subject has a steroid-induced myopathy. In some embodiments, the subject has an immunosuppressant-induced myopathy. In some embodiments, the subject has a chemotherapeutic-induced myopathy. In some embodiments, the subject has an alcohol-induced myopathy. In some embodiments, the method includes administering to a subject in need thereof an effective amount of the composition to treat immobilization. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat malnutrition. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat fasting. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat aging. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat autophagy. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat reduced protein synthesis. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat anabolic resistance. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat junction integrity (e.g., neuromuscular junction integrity). In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat insulin resistance. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat decreased mitochondrial biogenesis. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat anaplerosis. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

In some embodiments, the method includes administering to a subject in need thereof an effective amount of the composition to treat an energy deficit. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

The present disclosure also provides methods for enhancing muscle function that include administering to a subject in need thereof an effective amount of a composition including defined amino acid components. In some embodiments, the subject has or is identified as having decreased muscle function due to aging, injury, atrophy, infection, or disease. In some embodiments, the composition reduces muscle atrophy in the subject.

In some embodiments, the subject has or is identified as having muscle deterioration, decay, atrophy, cachexia, sarcopenia, drug-induced myopathy, muscular dystrophy, or myopenia. In some embodiments, the subject is a human. In some embodiments, the subject has not received prior treatment with a composition including defined amino acid components (e.g., a naïve subject).

In some embodiments, the subject has or is identified as having muscle deterioration. In some embodiments, the subject is a human. In some embodiments, the subject has not received prior treatment with a composition including defined amino acid components (e.g., a naïve subject).

In some embodiments, the subject has or is identified as having muscle decay. In some embodiments, the subject is a human. In some embodiments, the subject has not received prior treatment with a composition including defined amino acid components (e.g., a naïve subject).

In some embodiments, the subject has or is identified as having muscle atrophy. In some embodiments, the subject is a human. In some embodiments, the subject has not received prior treatment with a composition including defined amino acid components (e.g., a naïve subject).

In some embodiments, the subject has or is identified as having cachexia. In some embodiments, the subject is a human. In some embodiments, the subject has not received prior treatment with a composition including defined amino acid components (e.g., a naïve subject).

In some embodiments, the subject has or is identified as having sarcopenia. In some embodiments, the subject is a human. In some embodiments, the subject has not received prior treatment with a composition including defined amino acid components (e.g., a naïve subject).

In some embodiments, the subject has or is identified as having drug-induced myopathy. In some embodiments, the subject is a human. In some embodiments, the subject has not received prior treatment with a composition including defined amino acid components (e.g., a naïve subject).

In some embodiments, the subject has or is identified as having muscular dystrophy. In some embodiments, the subject is a human. In some embodiments, the subject has not received prior treatment with a composition including defined amino acid components (e.g., a naïve subject).

In some embodiments, the subject has or is identified as having myopenia. In some embodiments, the subject is a human. In some embodiments, the subject has not received prior treatment with a composition including defined amino acid components (e.g., a naïve subject).

In some embodiments, the subject has muscle weakness, e.g., muscle weakness of one, two, three, or more (e.g., all) of skeletal muscle, cardiac muscle, or smooth muscle. In certain embodiments, the subject has muscle weakness in one, two, three, four, five, six, or more (e.g., all) of a neck muscle, a torso muscle, an arm muscle, a shoulder muscle, a hand muscle, a leg muscle, or a foot muscle.

In some embodiments, the subject has had a surgery, e.g., rotator cuff surgery, knee surgery, or hip surgery, or has worn a cast prior to administration of the composition. In an embodiment, the subject has had rotator cuff surgery prior to administration of the composition. In an embodiment, the subject has had a knee surgery prior to administration of the composition. In an embodiment, the subject has had a hip surgery prior to administration of the composition. In an embodiment, the subject has worn a cast prior to administration of the composition.

In some embodiments, the subject has perceived muscle weakness, e.g., chronic fatigue syndrome.

In some embodiments, the subject has a cancer-associated muscle weakness.

In some embodiments, the subject has a neuromuscular disorder, e.g., myasthenia gravis or Lambert-Eaton myasthenic syndrome.

In some embodiments, the subject has muscular dystrophy, e.g., Duchenne muscular dystrophy, Becker muscular dystrophy, facioscapulohumeral muscular dystrophy, or myotonic dystrophy. In some embodiments, the subject has inflammatory myopathy, e.g., polymyositis or dermatomyositis.

In some embodiments, the subject has one, two, or more (e.g., all) of low sodium levels (e.g., hyponatremia), low potassium levels (e.g., hypokalemia), or a calcium deficiency or relatively high calcium levels (e.g., hypercalcemia).

In some embodiments, the subject has muscle weakness associated with nerve damage, e.g., neuralgia or peripheral neuropathy. In some embodiments, the subject has a bone weakness disease, e.g., osteomalacia, osteogenesis imperfecta, rickets, or Hypophosphatasia.

In some embodiments, the subject has experienced a stroke or a transient ischemic attack. In some embodiments, the subject has an autoimmune disease, e.g., Graves' disease.

In some embodiments, the subject has hypothyroidism. In some embodiments, the subject has amyotrophic lateral sclerosis (ALS). In some embodiments, administering the composition results in an improvement in one or more metabolic symptoms in the subject. In certain embodiments, the one or more metabolic symptoms is selected from the following: mTORC1 activation; improved insulin sensitivity; activation of muscle protein synthesis; scavenging of reactive oxygen species (ROS); decreased inflammation; inhibition catabolism; ammonia detoxification; and decreased fibrosis progression.

In some embodiments, the composition reduces muscle atrophy.

In some embodiments, the composition results in anabolism and catabolism of muscle tissue in the subject.

In some embodiments, administering the composition results in mTORC1 activation in the subject. In some embodiments, the composition also reduces muscle atrophy.

In some embodiments, administering the composition results in improved insulin sensitivity in the subject. In some embodiments, the composition also reduces muscle atrophy.

In some embodiments, administering the composition results in activation of muscle protein synthesis in the subject. In some embodiments, the composition also reduces muscle atrophy.

In some embodiments, administering the composition results in scavenging of reactive oxygen species (ROS) in the subject. In some embodiments, the composition also reduces muscle atrophy.

In some embodiments, administering the composition results in decreased inflammation in the subject. In some embodiments, the composition also reduces muscle atrophy.

In some embodiments, administering the composition results inhibited catabolism in the subject. In some embodiments, the composition also reduces muscle atrophy.

In some embodiments, administering the composition results in ammonia detoxification in the subject. In some embodiments, the composition also reduces muscle atrophy.

In some embodiments, administering the composition results in decreased fibrosis progression in the subject. In some embodiments, the composition also reduces muscle atrophy.

In some embodiments, the composition results in an improvement in one or both of muscle loss or muscle function related to one or both of immobilization or muscle disuse following injury in a subject. In some embodiments, the subject has had a surgery, e.g., rotator cuff surgery, knee surgery, or hip surgery, or has worn a cast, prior to administration of the composition. In some embodiments, the subject has had a hip fracture-related myopenia. In some embodiments, the subject has had a joint replacement. In some embodiments, the subject has had an injury repair surgery.

In some embodiments, the subject has ventilator-induced diaphragmatic dystrophy or ventilator-induced diaphragmatic dysfunction. In some embodiments, the subject has had one or both of ICU-acquired or burns-related myopathies.

In some embodiments, the subject has disease-related cachexia, e.g., a disease-related cachexia selected from chronic obstructive pulmonary disease (COPD), congestive heart failure (CHF), chronic kidney disease (CKD), and cancer.

In some embodiments, the composition is administered with a second agent.

The present disclosure also provides a method for reducing muscle atrophy comprising administering to a subject in need thereof an effective amount of a composition described herein.

The present disclosure also provides a composition described herein for use as a medicament.

The present disclosure provides a composition described herein for use as a medicament in enhancing muscle function.

The present disclosure provides a composition described herein for use as a medicament for treating one or more symptoms selected from the group consisting of immobilization, malnutrition, fasting, aging, autophagy, reduced protein synthesis, anabolic resistance, neuromuscular junction integrity, insulin resistance, decreased mitochondrial biogenesis, and anaplerosis.

The present disclosure provides a composition described herein for use in the manufacture of a medicament for enhancing muscle function. The present disclosure provides a use of a composition for the manufacture of a medicament for treating one or more symptoms selected from the group consisting of immobilization, malnutrition, fasting, aging, autophagy, reduced protein synthesis, anabolic resistance, neuromuscular junction integrity, insulin resistance, decreased mitochondrial biogenesis, and anaplerosis.

### Dosage Regimens

The composition can be administered according to a dosage regimen described herein to, e.g., enhance muscle function in a subject (e.g., a human, such as a human with muscle atrophy). The composition can be administered according to a dosage regimen described herein to treat (e.g., inhibit, reduce, ameliorate, or prevent) a disorder, e.g., a muscle disease in a subject (e.g., a human). In some embodiments, the subject has a rare muscle disease. In some embodiments, the the subject has muscle atrophy, sarcopenia, muscle deterioration, muscle decay, cachexia, drug-induced myopathy, muscular dystrophy, or myopenia. In some embodiments, the subject has a fracture or other trauma. In some embodiments, the subject has a statin-induced myopathy. In some embodiments, the subject has a steroid-induced myopathy. In some embodiments, the subject has an immunosuppressant-induced myopathy. In some embodiments, the subject has a chemotherapeutic-induced myopathy. In some embodiments, the subject has an alcohol-induced myopathy.

In some embodiments, the composition can be provided to a patient to enhance muscle function and/or treat a muscle disease or disorder (e.g., muscle atrophy, sarcopenia, muscle deterioration, muscle decay, cachexia, drug-induced myopathy, muscular dystrophy, or myopenia) in a patient in either a single or multiple dosage regimens. In some embodiments, doses can be administered, e.g., twice daily, three times daily, four times daily, five times daily, six times daily, seven times daily, or more. In some embodiments, the composition is administered for at least 2 days, 3 days, 4 days, 5 days, 6 days, 7 days, or 2 weeks. In some embodiments, the composition is administered for at least 10 weeks, 11 weeks, 12 weeks, 13 weeks, 14 weeks, 15 weeks, 16 weeks, 17 weeks, 18 weeks, 19 weeks, 20 weeks, or longer. In some embodiments, the composition can be administered chronically, e.g., more than 30 days, e.g., 31 days, 40 days, 50 days, 60 days, 3 months, 6 months, 9 months, one year, two years, or three years).

In some embodiments, the composition is administered at a dose of about 4 g and about 80 g total amino acids, e.g., once per day, twice per day, three times per day, four times per day, five times per day, or six times per day (e.g., three times per day). In some embodiments, the composition is administered at a dose of about 5 g to about 15 g, about 10 g to about 20 g, about 20 g to about 40 g, or about 30 g to about 50 g total amino acids, e.g., once per day, twice per day, three times per day, four times per day, five times per day, or six times per day (e.g., three times per day).

In some embodiments, the composition is administered at a dose of about 5 g to about 15 g (e.g., about 6 g total amino acids), e.g., once per day, twice per day, three times per day, four times per day, five times per day, or six times per day (e.g., three times per day). In an embodiment, about 18 g total amino acids is administered per day to enhance muscle function in a subject (e.g., the subject has or is identified as having decreased muscle function due to aging, injury, atrophy, infection, or disease).

In some embodiments, the composition is administered at a dose of about 5 g to about 15 g (e.g., about 6 g total amino acids), e.g., once per day, twice per day, three times per day, four times per day, five times per day, or six times per day (e.g., three times per day). In an embodiment, about 18 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject. In an embodiment, about 23 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject. In an embodiment, about 48 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject. In an embodiment, about 68 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject In an embodiment, about 72 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject.

In some embodiments, the composition is administered at a dose of about 15 g to about 40 g (e.g., about 24 g total amino acids), e.g., once per day, twice per day, three times per day, four times per day, five times per day, or six times per day (e.g., three times per day). Thus, about 68 g or about 72 g total amino acids is administered per day to enhance muscle function in a subject (e.g., the subject has or is identified as having decreased muscle function due to aging, injury, atrophy, infection, or disease).

In some embodiments, the composition is administered at a dose of about 15 g to about 40 g (e.g., about 24 g total amino acids), e.g., once per day, twice per day, three times per day, four times per day, five times per day, or six times per day (e.g., three times per day). Thus, about 68 g or about 72 g total amino acids is administered per day to treat a muscle disease or disorder (e.g., muscle atrophy, sarcopenia, muscle deterioration, muscle decay, cachexia, drug-induced myopathy, muscular dystrophy, or myopenia) in a subject.

In some embodiments, the composition is administered every 2 hours, every 3 hours, every 4 hours, every 5 hours, every 6 hours, every 7 hours, every 8 hours, every 9 hours, or every 10 hours to enhance muscle function in a subject (e.g., the subject has or is identified as having decreased muscle function due to aging, injury, atrophy, infection, or disease).

In some embodiments, the composition is administered prior to a meal (e.g., one, two, or more (e.g., all) of breakfast, lunch, or dinner). In some embodiments, the composition is administered concurrent with a meal (e.g., one, two, or more (e.g., all) of breakfast, lunch, or dinner). In some embodiments, the composition is administered following a meal (e.g., one, two, or more (e.g., all) of breakfast, lunch, or dinner).

### Dietary Compositions

The composition including amino acid entities can be a dietary composition, e.g., chosen from a medical food, a functional food, or a supplement.

The composition including amino acid entities can be for use as a dietary composition, e.g., chosen from a medical food, a functional food, or a supplement. In some embodiments, the dietary composition is for use in a method comprising administering the composition to a subject.

In some embodiments, the composition is for use in treating a subject having or identified as having decreased muscle function due to aging, injury, atrophy, infection, or disease.

In some embodiments, the subject has or is identified as having muscle deterioration, muscle decay, muscle atrophy, cachexia, sarcopenia, steroid myopathy, or muscular dystrophy

In some embodiments, the subject has one or both of type 2 diabetes or a relatively high BMI.

In some embodiments, the composition promotes weight loss in the subject.

In some embodiments, administration of the dietary composition results in an improvement in one or more metabolic symptoms in the subject, e.g., one or more metabolic symptoms is selected from the following: increased free fatty acid and lipid metabolism, improved mitochondrial function, white adipose tissue (WAT) browning, decreased reactive oxygen species (ROS), increased levels of glutathione (GSH), decreased hepatic inflammation, decreased hepatocyte ballooning, improved gut barrier function, increased insulin secretion, or glucose tolerance. In certain embodiments, administration of the composition results in an improvement in one or more metabolic symptoms after a treatment period of 24 hours.

### Methods of Providing an Amino Acid to a Subject

The present disclosure features a method of providing amino acid entities to a subject comprising administering to the subject an effective amount of a composition described herein, e.g., a composition comprising a leucine (L)-amino acid entity, a arginine (R)-amino acid entity, a glutamine (Q)-amino acid entity; and an antioxidant or reactive oxygen species (ROS) scavenger, e.g., a N-acetylcysteine (NAC) entity, e.g., NAC. In some embodiments, at least one amino acid entity is not a peptide of more than 20 amino acid residues in length. In some embodiments, the composition further comprises one or more EAA-entities, e.g., one, two, three, or more (e.g., all) of a H-amino acid-entity, a K-amino acid-entity, a F-amino acid-entity, and a T-amino acid-entity.

The present disclosure also features a method of increasing one, two, three, or more (e.g., all) amino acid entities in a subject comprising administering to the subject an effective amount of the composition described herein. In some embodiments, administration of the composition results in an increase in the amino acid entities in one, two, three, or more (e.g., all) of blood, plasma, or serum of the subject, e.g., in a blood, plasma, or serum sample from the subject.

### Biomarkers

Any of the methods disclosed herein can include evaluating or monitoring the effectiveness of administering a composition described herein to a subject. In some embodiments, the subject is in need of muscle function enhancement (e.g., a subject having muscle deterioration, muscle decay, muscle atrophy, cachexia, sarcopenia, drug-induced myopathy, muscular dystrophy, or myopenia).

In some embodiments, the value of effectiveness to the composition in treating a subject comprises a measure of the levels of one, two, three, four, five, six, seven, eight, nine, ten, eleven, twelve, thirteen, fourteen, fifteen, or more (e.g., all) of the following:
a) myostatin;
b) myoglobin;
c) Cortisol-AM;
d) C-reactive protein;
e) insulin;
f) cytokines (e.g., one, two, three, four, five, six, or more (e.g., all of IL-1A RBM, IL-1RA, IL-1 RI, IL-1 RII, IL-12, IL-18, or MCP-1);
g) GDF-11;
h) P3NP;
i) IGF-1;
j) IGFBP1;
k) IGFBP3;
l) FGF21;
m) DHEAS;
n) mTORC1;
o) Gcn2; or
p) AMP-activated protein kinase (AMPK).

In some embodiments of any of the methods disclosed herein, the measure of one or more of a)-p) is obtained from a sample acquired from the subject, e.g., a subject in need of muscle function enhancement (e.g., a subject having muscle deterioration, muscle decay, muscle atrophy, cachexia, sarcopenia, drug-induced myopathy, muscular dystrophy, or myopenia). In some embodiments, the sample is chosen from a blood sample (e.g., a plasma sample) or a muscle sample.

In some embodiments, the subject is evaluated prior to receiving, during, or after receiving, the composition.

In some embodiments, administration of the composition (e.g., at a dose of about 4 g to about 80 g total amino acids, e.g., about 6 g, about 12 g, about 18 g, or about 24 g three times daily), results in an improvement of one, two, three, four, five, six, seven, eight, nine, ten, eleven, twelve, thirteen, fourteen, fifteen, or more (e.g., all) of the following:
a) myostatin;
b) myoglobin;
c) Cortisol-AM;
d) C-reactive protein;
e) insulin;
f) cytokines (e.g., one, two, three, four, five, six, or more (e.g., all of IL-1A RBM, IL-1RA, IL-1 RI, IL-1 RII, IL-12, IL-18, or MCP-1);
g) GDF-11;
h) P3NP;
i) IGF-1;
j) IGFBP1;
k) IGFBP3;
l) FGF21;
m) DHEAS;
n) mTORC1;
o) Gcn2; or
p) AMP-activated protein kinase (AMPK).

In some embodiments, administration of the composition to the subject results in a decrease in levels of one, two, three, four, five, six, or more (e.g., all) of myoglobin, myostatin, GDF-11, cortisol-AM, C-reactive protein, insulin, or cytokines (e.g., one, two, three, four, five, six, or more (e.g., all of IL-1A RBM, IL-1RA, IL-1 RI, IL-1 RII, IL-12, IL-18, or MCP-1) in the subject (Table 4). In some embodiments, administration of the composition to the subject results in an increase in levels of one, two, three, four, five, six, or more (e.g., all) of P3NP, IGF-1, IGFBP1, IGFBP3, FGF-21, DHEAS, or mTORC1 in the subject (Table 4).

TABLE 4
Biomarkers to determine effect of the composition on muscle biology.

| | | Expected Change | |
|---|---|---|---|
| | | in Response to | Additional information regarding biomarker change on |
| Biomarker | Category | Composition | muscle synthesis and/or breakdown |
| Myoglobin | Muscle | Down | Decrease suggests a reduction in muscle breakdown and |
| | biology | | autophagy |
| Myostatin, GDF-11 | Muscle | Down | Myostatin act to inhibit muscle synthesis - decrease in levels |
| | biology | | indicate increase anabolism |
| | | | Change in GDF-11 levels to further inform changes to muscle |
| | | | biology |
| P3NP | Muscle | Up | P3NP is released during collagen synthesis in muscle |
| | biology | | Increased circulating P3NP indicates muscle growth, muscle |
| | | | repair and fibrosis |
| Cortisol-AM | Endocrine | Down | Endocrine molecules involved in regulating protein synthesis |
| C-reactive protein | Endocrine | Down | as stimulators/potentiators or inhibitors |
| IGF-1, IGFBP1, | Endocrine | Up | Increase in potentiator levels and decrease in inhibitor levels |
| IGFBP3, FGF21, | | | are supportive of net anabolism |
| DHEAS | | | |
| Insulin | Endocrine | Down | Decrease indicates moderation in insulin resistance, and |
| | (glucose | | increased glucose handling and anabolic sensitivity |
| | tolerance) | | |
| IL1ARBM, | Inflammation | Down | Increased muscle wasting is associated with a strong |
| IL1RA, IL1RI, | | | inflammatory response |
| IL1RII, IL-12, IL- | | | Reduced levels of these inflammation biomarkers indicate |
| 18, MCP-1, | | | reduction in inflammation |
| cytokines | | | Overall profile of these biomarker can further provide |
| | | | dynamic assessment on interleukin response to the |
| | | | composition |

In some embodiments, administration of the composition (e.g., at a dose of about 4 g and about 80 g total amino acids, e.g., about 6 g, about 12 g, about 18 g, or about 24 g three times daily), results in an improvement in one, two, three, four, five, or more (e.g., all) of a)-f) after a treatment period of, about 24 hours, about 72 hours, about 1 week, about 2 weeks, about 3 weeks, about 4 weeks, about 5 weeks, about 6 weeks, about 7 weeks, about 8 weeks, about 9 weeks, about 10 weeks, about 11 weeks, or 12 weeks. In certain embodiments, administration of the composition results in an improvement in one, two, three, four, five, six, seven, eight, nine, ten, eleven, twelve, thirteen, fourteen, fifteen, or more (e.g., all) of a)-r) after a treatment period of about 2 weeks.

### NUMBERED EMBODIMENTS

The invention is further described with reference to the following numbered embodiments.

1. A composition comprising:

a) a leucine (L)-amino acid entity, a arginine (R)-amino acid entity, and a glutamine (Q)-amino acid entity; and

b) an antioxidant or reactive oxygen species (ROS) scavenger, e.g., a N-acetylcysteine (NAC) entity, e.g., NAC; and optionally

c) an essential amino acid (EAA)-entity chosen from a histidine (H)-amino acid-entity, a lysine (K)-amino acid-entity, a phenylalanine (F)-amino acid-entity, and a threonine (T)-amino acid-entity or a combination of two, three, or four of the EAAs;

provided that:

d) at least one amino acid entity is not provided as a peptide of more than 20 amino acid residues in length, and optionally wherein:

(i) the amino acid entity of (a) is selected from Table 2; and

(ii) one or both of the R-amino acid entity and the Q-amino acid entity are present at a higher amount (wt. %) than the L-amino acid entity.

2. The composition of embodiment 1, wherein the composition comprises an amino acid and three amino acid entities.

3. The composition of embodiment 1, wherein the composition comprises an amino acid precursor and three amino acid entities.

4. The composition of embodiment 1, wherein the composition comprises an amino acid metabolite and three amino acid entities.

5. The composition of embodiment 1, wherein the composition comprises an amino acid derivative and three amino acid entities.

6. The composition of embodiment 1, wherein the composition comprises two amino acids and two amino acid entities.

7. The composition of embodiment 1, wherein the composition comprises two amino acid precursors and two amino acid entities.

8. The composition of embodiment 1, wherein the composition comprises two amino acid metabolites and two amino acid entities.

9. The composition of embodiment 1, wherein the composition comprises two amino acid derivatives and two amino acid entities.

10. The composition of embodiment 1, wherein the composition comprises three amino acids and one amino acid entity.

11. The composition of embodiment 1, wherein the composition comprises three amino acid precursors and one amino acid entity.

12. The composition of embodiment 1, wherein the composition comprises three amino acid metabolites and one amino acid entity.

13. The composition of embodiment 1, wherein the composition comprises three amino acid derivatives and one amino acid entity.

14. The composition of embodiment 1 or 2, wherein the composition comprises L-leucine, a R-amino acid entity, and a Q-amino acid entity, and an antioxidant or ROS scavenger, e.g., a NAC entity.

15. The composition of embodiment 1, 2, 14, or 380, wherein the composition comprises L-leucine, R-arginine, a Q-amino acid entity, and an antioxidant or ROS scavenger, e.g., a NAC entity.

16. The composition of embodiment 1, 2, 14, or 381, wherein the composition comprises L-leucine, argininosuccinate, a Q-amino acid entity, and an antioxidant or ROS scavenger, e.g., a NAC entity.

17. The composition of embodiment 1, 2, 14, or 382, wherein the composition comprises L-leucine, citrulline, a Q-amino acid entity, and an antioxidant or ROS scavenger, e.g., a NAC entity.

18. The composition of embodiment 1, 2, 14, or 383, wherein the composition comprises L-leucine, aspartate, a Q-amino acid entity, and an antioxidant or ROS scavenger, e.g., a NAC entity.

19. The composition of embodiment 1, 2, 14, or 384, wherein the composition comprises L-leucine, L-glutamate, a Q-amino acid entity, and an antioxidant or ROS scavenger, e.g., a NAC entity.

20. The composition of embodiment 1, 2, 14, or 385, wherein the composition comprises L-leucine, ornithine, a Q-amino acid entity, and an antioxidant or ROS scavenger, e.g., a NAC entity.

21. The composition of embodiment 1, 2, 14, or 386, wherein the composition comprises a L-leucine, agmatine, a Q-amino acid entity, and an antioxidant or ROS scavenger, e.g., a NAC entity.

22. The composition of embodiment 1, 2, 14, or 387, wherein the composition comprises a L-leucine, creatine, a Q-amino acid entity, and an antioxidant or ROS scavenger, e.g., a NAC entity.

23. The composition of embodiment 1, 2, 14, or 388, wherein the composition comprises L-leucine, D-arginine, a Q-amino acid entity, and an antioxidant or ROS scavenger, e.g., a NAC entity.

24. The composition of embodiment 1, 2, 14, or 389, wherein the composition comprises L-leucine, N-acetyl-arginine, a Q-amino acid entity, and an antioxidant or ROS scavenger, e.g., a NAC entity.

25. The composition of embodiment 1, 2, 14, or 428, wherein the composition comprises L-leucine, a R-amino acid entity, L-glutamine, and an antioxidant or ROS scavenger, e.g., a NAC entity.

26. The composition of embodiment 1, 2, 14, or 429, wherein the composition comprises L-leucine, a R-amino acid entity, glutamate, and an antioxidant or ROS scavenger, e.g., a NAC entity.

27. The composition of embodiment 1, 2, 14, or 430, wherein the composition comprises L-leucine, a R-amino acid entity, carbamoyl-P, and an antioxidant or ROS scavenger, e.g., a NAC entity.

28. The composition of embodiment 1, 2, 14, or 431, wherein the composition comprises L-leucine, a R-amino acid entity, D-glutamine, and an antioxidant or ROS scavenger, e.g., a NAC entity.

29. The composition of embodiment 1, 2, 14, or 432, wherein the composition comprises L-leucine, a R-amino acid entity, N-acetyl-glutamine, and an antioxidant or ROS scavenger, e.g., a NAC entity.

30. The composition of embodiment 1, 2, 14, or 445, wherein the composition comprises L-leucine, a R-amino acid entity, a Q-amino acid entity, and NAC.

31. The composition of embodiment 1, 2, 14, or 446, wherein the composition comprises L-leucine, a R-amino acid entity, a Q-amino acid entity, and serine.

32. The composition of embodiment 1, 2, 14, or 447, wherein the composition comprises L-leucine, a R-amino acid entity, a Q-amino acid entity, and acetylserine.

33. The composition of embodiment 1, 2, 14, or 448, wherein the composition comprises L-leucine, a R-amino acid entity, a Q-amino acid entity, and cystathionine.

34. The composition of embodiment 1, 2, 14, or 449, wherein the composition comprises L-leucine, a R-amino acid entity, a Q-amino acid entity, and glutathione.

35. The composition of embodiment 1, 2, 14, or 450, wherein the composition comprises L-leucine, a R-amino acid entity, a Q-amino acid entity, and homocysteine.

36. The composition of embodiment 1, 2, 14, or 451, wherein the composition comprises L-leucine, a R-amino acid entity, a Q-amino acid entity, and methionine.

37. The composition of embodiment 1, 2, 14, or 452, wherein the composition comprises L-leucine, a R-amino acid entity, a Q-amino acid entity, and D-cysteine.

38. The composition of embodiment 1, 2, 14, or 453, wherein the composition comprises L-leucine, a R-amino acid entity, a Q-amino acid entity, and L-cysteine.

39. The composition of embodiment 1, 2, 14, or 454, wherein the composition comprises L-leucine, a R-amino acid entity, a Q-amino acid entity, and cystine.

40. The composition of embodiment 1, 2, 14, 380, or 428, wherein the composition comprises L-leucine, L-arginine, L-glutamine, and an antioxidant or ROS scavenger, e.g., a NAC entity.

41. The composition of embodiment 1, 2, 14, 381, or 429, wherein the composition comprises L-leucine, argininosuccinate, glutamate, and an antioxidant or ROS scavenger, e.g., a NAC entity.

42. The composition of embodiment 1, 2, 14, 382, or 431, wherein the composition comprises L-leucine, citrulline, D-glutamine, and an antioxidant or ROS scavenger, e.g., a NAC entity.

43. The composition of embodiment 1, 2, 14, or 383, wherein the composition comprises L-leucine, aspartate, N-acetyl-glutamine, and an antioxidant or ROS scavenger, e.g., a NAC entity.

44. The composition of embodiment 1, 2, 14, 380, or 445, wherein the composition comprises L-leucine, L-arginine, a Q-amino acid entity, and NAC.

45. The composition of embodiment 1, 2, 14, 381, or 446, wherein the composition comprises L-leucine, argininosuccinate, a Q-amino acid entity, and serine.

46. The composition of embodiment 1, 2, 14, 382, or 447, wherein the composition comprises L-leucine, citrulline, a Q-amino acid entity, and acetylserine.

47. The composition of embodiment 1, 2, 14, 383, or 448, wherein the composition comprises L-leucine, aspartate, a Q-amino acid entity, and cystathionine.

48. The composition of embodiment 1, 2, 14, 384, or 449, wherein the composition comprises L-leucine, glutamate, a Q-amino acid entity, and glutathione.

49. The composition of embodiment 1, 2, 14, 385, or 450, wherein the composition comprises L-leucine, ornithine, a Q-amino acid entity, and homocysteine.

50. The composition of embodiment 1, 2, 14, 386, or 451, wherein the composition comprises L-leucine, agmatine, a Q-amino acid entity, and methionine.

51. The composition of embodiment 1, 2, 14, 387, or 452, wherein the composition comprises L-leucine, creatine, a Q-amino acid entity, and D-cysteine.

52. The composition of embodiment 1, 2, 14, 388, or 453, wherein the composition comprises L-leucine, D-arginine, a Q-amino acid entity, and L-cysteine.

53. The composition of embodiment 1, 2, 14, 389, or 454, wherein the composition comprises L-leucine, N-acetyl-arginine, a Q-amino acid entity, and cystine.

54. The composition of embodiment 1, 2, 14, 428, or 445, wherein the composition comprises L-leucine, a R-amino acid entity, L-glutamine, and NAC.

55. The composition of embodiment 1, 2, 14, 429, or 446, wherein the composition comprises L-leucine, a R-amino acid entity, glutamate, and serine.

56. The composition of embodiment 1, 2, 14, 430, or 447, wherein the composition comprises L-leucine, a R-amino acid entity, carbamoyl-P, and acetylserine.

57. The composition of embodiment 1, 2, 14, 432, or 448, wherein the composition comprises L-leucine, a R-amino acid entity, N-acetyl-glutamine, and cystathionine.

58. The composition of embodiment 1, 2, 14, 433, or 449, wherein the composition comprises L-leucine, a R-amino acid entity, L-glutamine, and glutathione.

59. The composition of embodiment 1, 2, 14, or 450, wherein the composition comprises L-leucine, a R-amino acid entity, glutamate, and homocysteine.

60. The composition of embodiment 1, 2, 14, or 451, wherein the composition comprises L-leucine, a R-amino acid entity, carbamoyl-P, and methionine.

61. The composition of embodiment 1, 2, 14, or 452, wherein the composition comprises L-leucine, a R-amino acid entity, N-acetyl-glutamine, and D-cysteine.

62. The composition of embodiment 1, 2, 14, or 453, wherein the composition comprises L-leucine, a R-amino acid entity, L-glutamine, and L-cysteine.

63. The composition of embodiment 1, 2, 14, or 454, wherein the composition comprises L-leucine, a R-amino acid entity, a glutamate, and cystine.

64. The composition of embodiment 1, 2, 14, 380, or 445, wherein the composition comprises L-leucine, L-arginine, L-glutamine, and NAC.

65. The composition of embodiment 1, 2, 14, 381, or 446, wherein the composition comprises L-leucine, argininosuccinate, glutamate, and serine.

66. The composition of embodiment 1, 2, 14, 382, or 447, wherein the composition comprises L-leucine, citrulline, carbamoyl-P, and acetylserine.

67. The composition of embodiment 1, 2, 14, 383, or 448, wherein the composition comprises L-leucine, aspartate, D-glutamine, and cystathionine.

68. The composition of embodiment 1, 2, 14, 384, or 449, wherein the composition comprises L-leucine, glutamate, L-glutamine, and glutathione.

69. The composition of embodiment 1, 2, 14, 385, or 450, wherein the composition comprises L-leucine, ornithine, glutamate, and homocysteine.

70. The composition of embodiment 1, 2, 14, 386, or 451, wherein the composition comprises L-leucine, agmatine, carbamoyl-P, and methionine.

71. The composition of embodiment 1, 2, 14, 387, or 452, wherein the composition comprises L-leucine, creatine, D-glutamine and D-cysteine.

72. The composition of embodiment 1, 2, 14, 388, or 453, wherein the composition comprises L-leucine, D-arginine, a Q-amino acid entity, and L-cysteine.

73. The composition of embodiment 1, 2, 14, 389, or 454, wherein the composition comprises L-leucine, N-acetyl-arginine, argininosuccinate, and cystine.

74. The composition of embodiment 1 or 3, wherein the composition comprises oxo-leucine, a R-amino acid entity, a Q-amino acid entity, and an antioxidant or ROS scavenger, e.g., a NAC entity.

75. The composition of embodiment 1, 3, or 74, wherein the composition comprises oxo-leucine, a R-amino acid entity, and a Q-amino acid entity, and an antioxidant or ROS scavenger, e.g., a NAC entity.

76. The composition of embodiment 1, 3, or 74, wherein the composition comprises oxo-leucine, L-arginine, a Q-amino acid entity, and an antioxidant or ROS scavenger, e.g., a NAC entity.

77. The composition of embodiment 1, 3, or 74, wherein the composition comprises oxo-leucine, argininosuccinate, a Q-amino acid entity, and an antioxidant or ROS scavenger, e.g., a NAC entity.

78. The composition of embodiment 1, 3, or 74, wherein the composition comprises oxo-leucine, citrulline, a Q-amino acid entity, and an antioxidant or ROS scavenger, e.g., a NAC entity.

79. The composition of embodiment 1, 3, or 74, wherein the composition comprises oxo-leucine, aspartate, a Q-amino acid entity, and an antioxidant or ROS scavenger, e.g., a NAC entity.

80. The composition of embodiment 1, 3, or 74, wherein the composition comprises oxo-leucine, glutamate, a Q-amino acid entity, and an antioxidant or ROS scavenger, e.g., a NAC entity.

81. The composition of embodiment 1, 3, or 74, wherein the composition comprises oxo-leucine, ornithine, a Q-amino acid entity, and an antioxidant or ROS scavenger, e.g., a NAC entity.

82. The composition of embodiment 1, 3, or 74, wherein the composition comprises oxo-leucine, agmatine, a Q-amino acid entity, and an antioxidant or ROS scavenger, e.g., a NAC entity.

83. The composition of embodiment 1, 3, or 74, wherein the composition comprises oxo-leucine, creatine, a Q-amino acid entity, and an antioxidant or ROS scavenger, e.g., a NAC entity.

84. The composition of embodiment 1, 3, or 74, wherein the composition comprises oxo-leucine, D-arginine, a Q-amino acid entity, and an antioxidant or ROS scavenger, e.g., a NAC entity.

85. The composition of embodiment 1, 3, or 74, wherein the composition comprises oxo-leucine, N-acetyl-arginine, a Q-amino acid entity, and an antioxidant or ROS scavenger, e.g., a NAC entity.

86. The composition of embodiment 1, 3, or 74, wherein the composition comprises oxo-leucine, a R-amino acid entity, L-glutamine, and an antioxidant or ROS scavenger, e.g., a NAC entity.

87. The composition of embodiment 1, 3, or 74, wherein the composition comprises oxo-leucine, a R-amino acid entity, glutamate, and an antioxidant or ROS scavenger, e.g., a NAC entity.

88. The composition of embodiment 1, 3, or 74, wherein the composition comprises oxo-leucine, a R-amino acid entity, carbamoyl-P, and an antioxidant or ROS scavenger, e.g., a NAC entity.

89. The composition of embodiment 1, 3, or 74, wherein the composition comprises oxo-leucine, a R-amino acid entity, D-glutamine, and an antioxidant or ROS scavenger, e.g., a NAC entity.

90. The composition of embodiment 1, 3, or 74, wherein the composition comprises oxo-leucine, a R-amino acid entity, N-acetyl-glutamine, and an antioxidant or ROS scavenger, e.g., a NAC entity.

91. The composition of embodiment 1, 3, or 74, wherein the composition comprises oxo-leucine, a R-amino acid entity, a Q-amino acid entity, and NAC.

92. The composition of embodiment 1, 3, or 74, wherein the composition comprises oxo-leucine, a R-amino acid entity, a Q-amino acid entity, and serine.

93. The composition of embodiment 1, 3, or 74, wherein the composition comprises oxo-leucine, a R-amino acid entity, a Q-amino acid entity, and acetylserine.

94. The composition of embodiment 1, 3, or 74, wherein the composition comprises oxo-leucine, a R-amino acid entity, a Q-amino acid entity, and cystathionine.

95. The composition of embodiment 1, 3, or 74, wherein the composition comprises oxo-leucine, a R-amino acid entity, a Q-amino acid entity, and glutathione.

96. The composition of embodiment 1, 3, or 74, wherein the composition comprises oxo-leucine, a R-amino acid entity, a Q-amino acid entity, and homocysteine.

97. The composition of embodiment 1, 3, or 74, wherein the composition comprises oxo-leucine, a R-amino acid entity, a Q-amino acid entity, and methionine.

98. The composition of embodiment 1, 3, or 74, wherein the composition comprises oxo-leucine, a R-amino acid entity, a Q-amino acid entity, and D-cysteine.

99. The composition of embodiment 1, 3, or 74, wherein the composition comprises oxo-leucine, a R-amino acid entity, a Q-amino acid entity, and L-cysteine.

100. The composition of embodiment 1, 3, or 74, wherein the composition comprises oxo-leucine, a R-amino acid entity, a Q-amino acid entity, and a NAC entity.

101. The composition of embodiment 1, 3, or 74, wherein the composition comprises oxo-leucine, a R-amino acid entity, a Q-amino acid entity, and cystine.

102. The composition of embodiment 1, 3, or 74, wherein the composition comprises oxo-leucine, L-arginine, L-glutamine, and an antioxidant or ROS scavenger, e.g., a NAC entity.

103. The composition of embodiment 1, 3, or 74, wherein the composition comprises oxo-leucine, argininosuccinate, glutamate, and an antioxidant or ROS scavenger, e.g., a NAC entity.

104. The composition of embodiment 1, 3, or 74, wherein the composition comprises oxo-leucine, citrulline, D-glutamine, and an antioxidant or ROS scavenger, e.g., a NAC entity.

105. The composition of embodiment 1, 3, or 74,wherein the composition comprises oxo-leucine, aspartate, N-acetyl-glutamine, and an antioxidant or ROS scavenger, e.g., a NAC entity.

106. The composition of embodiment 1, 3, or 74, wherein the composition comprises oxo-leucine, L-arginine, a Q-amino acid entity, and NAC.

107. The composition of embodiment 1, 3, or 74, wherein the composition comprises oxo-leucine, argininosuccinate, a Q-amino acid entity, and serine.

108. The composition of embodiment 1, 3, or 74, wherein the composition comprises oxo-leucine, citrulline, a Q-amino acid entity, and acetylserine.

109. The composition of embodiment 1, 3, or 74, wherein the composition comprises oxo-leucine, aspartate, a Q-amino acid entity, and cystathionine.

110. The composition of embodiment 1, 3, or 74, wherein the composition comprises oxo-leucine, glutamate, a Q-amino acid entity, and glutathione.

111. The composition of embodiment 1, 3, or 74, wherein the composition comprises oxo-leucine, ornithine, a Q-amino acid entity, and homocysteine.

112. The composition of embodiment 1, 3, or 74, wherein the composition comprises oxo-leucine, agmatine, a Q-amino acid entity, and methionine.

113. The composition of embodiment 1, 3, or 74, wherein the composition comprises oxo-leucine, creatine, a Q-amino acid entity, and D-cysteine.

114. The composition of embodiment 1, 3, or 74, wherein the composition comprises oxo-leucine, D-arginine, a Q-amino acid entity, and L-cysteine.

115. The composition of embodiment 1, 3, or 74, wherein the composition comprises oxo-leucine, N-acetyl-arginine, a Q-amino acid entity, and cystine.

116. The composition of embodiment 1, 3, or 74, wherein the composition comprises oxo-leucine, a R-amino acid entity, L-glutamine, and NAC.

117. The composition of embodiment 1, 3, or 74, wherein the composition comprises oxo-leucine, a R-amino acid entity, glutamate, and serine.

118. The composition of embodiment 1, 3, or 74, wherein the composition comprises oxo-leucine, a R-amino acid entity, carbamoyl-P, and acetylserine.

119. The composition of embodiment 1, 3, or 74, wherein the composition comprises oxo-leucine, a R-amino acid entity, N-acetyl-glutamine, and cystathionine.

120. The composition of embodiment 1, 3, or 74, wherein the composition comprises oxo-leucine, a R-amino acid entity, L-glutamine, and glutathione.

121. The composition of embodiment 1, 3, or 74, wherein the composition comprises oxo-leucine, a R-amino acid entity, glutamate, and homocysteine.

122. The composition of embodiment 1, 3, or 74, wherein the composition comprises oxo-leucine, a R-amino acid entity, carbamoyl-P, and methionine.

123. The composition of embodiment 1, 3, or 74, wherein the composition comprises oxo-leucine, a R-amino acid entity, N-acetyl-glutamine, and D-cysteine.

124. The composition of embodiment 1, 3, or 74, wherein the composition comprises oxo-leucine, a R-amino acid entity, L-glutamine, and L-cysteine.

125. The composition of embodiment 1, 3, or 74, wherein the composition comprises oxo-leucine, a R-amino acid entity, a glutamate, and cystine.

126. The composition of embodiment 1, 3, or 74, wherein the composition comprises oxo-leucine, L-arginine, L-glutamine, and NAC.

127. The composition of embodiment 1, 3, or 74, wherein the composition comprises oxo-leucine, argininosuccinate, glutamate, and serine.

128. The composition of embodiment 1, 3, or 74, wherein the composition comprises oxo-leucine, citrulline, carbamoyl-P, and acetylserine.

129. The composition of embodiment 1, 3, or 74, wherein the composition comprises oxo-leucine, aspartate, D-glutamine, and cystathionine.

130. The composition of embodiment 1, 3, or 74, wherein the composition comprises oxo-leucine, N-acetyl-glutamine, L-glutamine, and glutathione.

131. The composition of embodiment 1, 3, or 74, wherein the composition comprises oxo-leucine, ornithine, glutamate, and homocysteine.

132. The composition of embodiment 1, 3, or 74, wherein the composition comprises oxo-leucine, agmatine, carbamoyl-P, and methionine.

133. The composition of embodiment 1, 3, or 74, wherein the composition comprises oxo-leucine, creatine, D-glutamine and D-cysteine.

134. The composition of embodiment 1, 3, or 74, wherein the composition comprises oxo-leucine, D-arginine, a Q-amino acid entity, and L-cysteine.

135. The composition of embodiment 1, 3, or 74, wherein the composition comprises oxo-leucine, N-acetyl-arginine, argininosuccinate, and cystine.

136. The composition of embodiment 1 or 4, wherein the composition comprises HMB, a R-amino acid entity, a Q-amino acid entity, and an antioxidant or ROS scavenger, e.g., a NAC entity.

137. The composition of embodiment 1, 4, or 136, wherein the composition comprises HMB, L-arginine, a Q-amino acid entity, and an antioxidant or ROS scavenger, e.g., a NAC entity.

138. The composition of embodiment 1, 4, or 136, wherein the composition comprises HMB, argininosuccinate, a Q-amino acid entity, and an antioxidant or ROS scavenger, e.g., a NAC entity.

139. The composition of embodiment 1, 4, or 136, wherein the composition comprises HMB, citrulline, a Q-amino acid entity, and an antioxidant or ROS scavenger, e.g., a NAC entity.

140. The composition of embodiment 1, 4, or 136, wherein the composition comprises HMB, aspartate, a Q-amino acid entity, and an antioxidant or ROS scavenger, e.g., a NAC entity.

141. The composition of embodiment 1, 4, or 136, wherein the composition comprises HMB, glutamate, a Q-amino acid entity, and an antioxidant or ROS scavenger, e.g., a NAC entity.

142. The composition of embodiment 1, 4, or 136, wherein the composition comprises HMB, ornithine, a Q-amino acid entity, and an antioxidant or ROS scavenger, e.g., a NAC entity.

143. The composition of embodiment 1, 4, or 136, wherein the composition comprises HMB, agmatine, a Q-amino acid entity, and an antioxidant or ROS scavenger, e.g., a NAC entity.

144. The composition of embodiment 1, 4, or 136, wherein the composition comprises HMB, creatine, a Q-amino acid entity, and an antioxidant or ROS scavenger, e.g., a NAC entity.

145. The composition of embodiment 1, 4, or 136, wherein the composition comprises HMB, D-arginine, a Q-amino acid entity, and an antioxidant or ROS scavenger, e.g., a NAC entity.

146. The composition of embodiment 1, 4, or 136, wherein the composition comprises HMB, N-acetyl-arginine, a Q-amino acid entity, and an antioxidant or ROS scavenger, e.g., a NAC entity.

147. The composition of embodiment 1, 4, or 136, wherein the composition comprises HMB, a R-amino acid entity, L-glutamine, and an antioxidant or ROS scavenger, e.g., a NAC entity.

148. The composition of embodiment 1, 4, or 136, wherein the composition comprises HMB, a R-amino acid entity, glutamate, and an antioxidant or ROS scavenger, e.g., a NAC entity.

149. The composition of embodiment 1, 4, or 136, wherein the composition comprises HMB, a R-amino acid entity, carbamoyl-P, and an antioxidant or ROS scavenger, e.g., a NAC entity.

150. The composition of embodiment 1, 4, or 136, wherein the composition comprises HMB, a R-amino acid entity, D-glutamine, and an antioxidant or ROS scavenger, e.g., a NAC entity.

151. The composition of embodiment 1, 4, or 136, wherein the composition comprises HMB, a R-amino acid entity, N-acetyl-glutamine, and an antioxidant or ROS scavenger, e.g., a NAC entity.

152. The composition of embodiment 1, 4, or 136, wherein the composition comprises HMB, a R-amino acid entity, a Q-amino acid entity, and NAC.

153. The composition of embodiment 1, 4, or 136, wherein the composition comprises HMB, a R-amino acid entity, a Q-amino acid entity, and serine.

154. The composition of embodiment 1, 4, or 136, wherein the composition comprises HMB, a R-amino acid entity, a Q-amino acid entity, and acetylserine.

155. The composition of embodiment 1, 4, or 136, wherein the composition comprises HMB, a R-amino acid entity, a Q-amino acid entity, and cystathionine.

156. The composition of embodiment 1, 4, or 136, wherein the composition comprises HMB, a R-amino acid entity, a Q-amino acid entity, and glutathione.

157. The composition of embodiment 1, 4, or 136, wherein the composition comprises HMB, a R-amino acid entity, a Q-amino acid entity, and homocysteine.

158. The composition of embodiment 1, 4, or 136, wherein the composition comprises HMB, a R-amino acid entity, a Q-amino acid entity, and methionine.

159. The composition of embodiment 1, 4, or 136, wherein the composition comprises HMB, a R-amino acid entity, a Q-amino acid entity, and D-cysteine.

160. The composition of embodiment 1, 4, or 136, wherein the composition comprises HMB, a R-amino acid entity, a Q-amino acid entity, and L-cysteine.

161. The composition of embodiment 1, 4, or 136, wherein the composition comprises HMB, a R-amino acid entity, a Q-amino acid entity, and cysteine.

162. The composition of embodiment 1, 4, or 136, wherein the composition comprises HMB, a R-amino acid entity, a Q-amino acid entity, and cystine.

163. The composition of embodiment 1, 4, or 136, wherein the composition comprises HMB, L-arginine, L-glutamine, and an antioxidant or ROS scavenger, e.g., a NAC entity.

164. The composition of embodiment 1, 4, or 136, wherein the composition comprises HMB, argininosuccinate, glutamate, and an antioxidant or ROS scavenger, e.g., a NAC entity.

165. The composition of embodiment 1, 4, or 136, wherein the composition comprises HMB, citrulline, D-glutamine, and an antioxidant or ROS scavenger, e.g., a NAC entity.

166. The composition of embodiment 1, 4, or 136, wherein the composition comprises HMB, aspartate, N-acetyl-glutamine, and an antioxidant or ROS scavenger, e.g., a NAC entity.

167. The composition of embodiment 1, 4, or 136, wherein the composition comprises HMB, L-arginine, a Q-amino acid entity, and NAC.

168. The composition of embodiment 1, 4, or 136, wherein the composition comprises HMB, argininosuccinate, a Q-amino acid entity, and serine.

169. The composition of embodiment 1, 4, or 136, wherein the composition comprises HMB, citrulline, a Q-amino acid entity, and acetylserine.

170. The composition of embodiment 1, 4, or 136, wherein the composition comprises HMB, aspartate, a Q-amino acid entity, and cystathionine.

171. The composition of embodiment 1, 4, or 136, wherein the composition comprises HMB, glutamate, a Q-amino acid entity, and glutathione.

172. The composition of embodiment 1, 4, or 136, wherein the composition comprises HMB, ornithine, a Q-amino acid entity, and homocysteine.

173. The composition of embodiment 1, 4, or 136, wherein the composition comprises HMB, agmatine, a Q-amino acid entity, and methionine.

174. The composition of embodiment 1, 4, or 136, wherein the composition comprises HMB, creatine, a Q-amino acid entity, and D-cysteine.

175. The composition of embodiment 1, 4, or 136, wherein the composition comprises HMB, D-arginine, a Q-amino acid entity, and L-cysteine.

176. The composition of embodiment 1, 4, or 136, wherein the composition comprises HMB, N-acetyl-arginine, a Q-amino acid entity, and cystine.

177. The composition of embodiment 1, 4, or 136, wherein the composition comprises HMB, a R-amino acid entity, L-glutamine, and NAC.

178. The composition of embodiment 1, 4, or 136, wherein the composition comprises HMB, a R-amino acid entity, glutamate, and serine.

179. The composition of embodiment 1, 4, or 136, wherein the composition comprises HMB, a R-amino acid entity, carbamoyl-P, and acetylserine.

180. The composition of embodiment 1, 4, or 136, wherein the composition comprises HMB, a R-amino acid entity, N-acetyl-glutamine, and cystathionine.

181. The composition of embodiment 1, 4, or 136, wherein the composition comprises HMB, a R-amino acid entity, L-glutamine, and glutathione.

182. The composition of embodiment 1, 4, or 136, wherein the composition comprises HMB, a R-amino acid entity, glutamate, and homocysteine.

183. The composition of embodiment 1, 4, or 136, wherein the composition comprises HMB, a R-amino acid entity, carbamoyl-P, and methionine.

184. The composition of embodiment 1, 4, or 136, wherein the composition comprises HMB, a R-amino acid entity, N-acetyl-glutamine, and D-cysteine.

185. The composition of embodiment 1, 4, or 136, wherein the composition comprises HMB, a R-amino acid entity, L-glutamine, and L-cysteine.

186. The composition of embodiment 1, 4, or 136, wherein the composition comprises HMB, a R-amino acid entity, a glutamate, and cystine.

187. The composition of embodiment 1, 4, or 136, wherein the composition comprises HMB, L-arginine, L-glutamine, and NAC.

188. The composition of embodiment 1, 4, or 136, wherein the composition comprises HMB, argininosuccinate, glutamate, and serine.

189. The composition of embodiment 1, 4, or 136, wherein the composition comprises HMB, citrulline, carbamoyl-P, and acetylserine.

190. The composition of embodiment 1, 4, or 136, wherein the composition comprises HMB, aspartate, D-glutamine, and cystathionine.

191. The composition of embodiment 1, 4, or 136, wherein the composition comprises HMB, N-acetyl-glutamine, L-glutamine, and glutathione.

192. The composition of embodiment 1, 4, or 136, wherein the composition comprises HMB, ornithine, glutamate, and homocysteine.

193. The composition of embodiment 1, 4, or 136, wherein the composition comprises HMB, agmatine, carbamoyl-P, and methionine.

194. The composition of embodiment 1, 4, or 136, wherein the composition comprises HMB, creatine, D-glutamine and D-cysteine.

195. The composition of embodiment 1, 4, or 136, wherein the composition comprises HMB, D-arginine, a Q-amino acid entity, and L-cysteine.

196. The composition of embodiment 1, 4, or 136, wherein the composition comprises HMB, N-acetyl-arginine, argininosuccinate, and cystine.

197. The composition of embodiment 1, or 4, wherein the composition comprises isovaleryl-CoA, a R-amino acid entity, a Q-amino acid entity, and an antioxidant or ROS scavenger, e.g., a NAC entity.

198. The composition of embodiment 1, 4, or 197, wherein the composition comprises isovaleryl-CoA, L-arginine, a Q-amino acid entity, and an antioxidant or ROS scavenger, e.g., a NAC entity.

199. The composition of embodiment 1, 4, or 197, wherein the composition comprises isovaleryl-CoA, argininosuccinate, a Q-amino acid entity, and an antioxidant or ROS scavenger, e.g., a NAC entity.

200. The composition of embodiment 1, 4, or 197, wherein the composition comprises isovaleryl-CoA, citrulline, a Q-amino acid entity, and an antioxidant or ROS scavenger, e.g., a NAC entity.

201. The composition of embodiment 1, 4, or 197, wherein the composition comprises isovaleryl-CoA, aspartate, a Q-amino acid entity, and an antioxidant or ROS scavenger, e.g., a NAC entity.

202. The composition of embodiment 1, 4, or 197, wherein the composition comprises isovaleryl-CoA, glutamate, a Q-amino acid entity, and an antioxidant or ROS scavenger, e.g., a NAC entity.

203. The composition of embodiment 1, 4, or 197, wherein the composition comprises isovaleryl-CoA, ornithine, a Q-amino acid entity, and an antioxidant or ROS scavenger, e.g., a NAC entity.

204. The composition of embodiment 1, 4, or 197, wherein the composition comprises isovaleryl-CoA, agmatine, a Q-amino acid entity, and an antioxidant or ROS scavenger, e.g., a NAC entity.

205. The composition of embodiment 1, 4, or 197, wherein the composition comprises isovaleryl-CoA, creatine, a Q-amino acid entity, and an antioxidant or ROS scavenger, e.g., a NAC entity.

206. The composition of embodiment 1, 4, or 197, wherein the composition comprises isovaleryl-CoA, D-arginine, a Q-amino acid entity, and an antioxidant or ROS scavenger, e.g., a NAC entity.

207. The composition of embodiment 1, 4, or 197, wherein the composition comprises isovaleryl-CoA, N-acetyl-arginine, a Q-amino acid entity, and an antioxidant or ROS scavenger, e.g., a NAC entity.

208. The composition of embodiment 1, 4, or 197, wherein the composition comprises isovaleryl-CoA, a R-amino acid entity, L-glutamine, and an antioxidant or ROS scavenger, e.g., a NAC entity.

209. The composition of embodiment 1, 4, or 197, wherein the composition comprises isovaleryl-CoA, a R-amino acid entity, glutamate, and an antioxidant or ROS scavenger, e.g., a NAC entity.

210. The composition of embodiment 1, 4, or 197, wherein the composition comprises isovaleryl-CoA, a R-amino acid entity, carbamoyl-P, and an antioxidant or ROS scavenger, e.g., a NAC entity.

211. The composition of embodiment 1, 4, or 197, wherein the composition comprises isovaleryl-CoA, a R-amino acid entity, D-glutamine, and an antioxidant or ROS scavenger, e.g., a NAC entity.

212. The composition of embodiment 1, 4, or 197, wherein the composition comprises isovaleryl-CoA, a R-amino acid entity, N-acetyl-glutamine, and an antioxidant or ROS scavenger, e.g., a NAC entity.

213. The composition of embodiment 1, 4, or 197, wherein the composition comprises isovaleryl-CoA, a R-amino acid entity, a Q-amino acid entity, and NAC.

214. The composition of embodiment 1, 4, or 197, wherein the composition comprises isovaleryl-CoA, a R-amino acid entity, a Q-amino acid entity, and serine.

215. The composition of embodiment 1, 4, or 197, wherein the composition comprises isovaleryl-CoA, a R-amino acid entity, a Q-amino acid entity, and acetylserine.

216. The composition of embodiment 1, 4, or 197, wherein the composition comprises isovaleryl-CoA, a R-amino acid entity, a Q-amino acid entity, and cystathionine.

217. The composition of embodiment 1, 4, or 197, wherein the composition comprises isovaleryl-CoA, a R-amino acid entity, a Q-amino acid entity, and glutathione.

218. The composition of embodiment 1, 4, or 197, wherein the composition comprises isovaleryl-CoA, a R-amino acid entity, a Q-amino acid entity, and homocysteine.

219. The composition of embodiment 1, 4, or 197, wherein the composition comprises isovaleryl-CoA, a R-amino acid entity, a Q-amino acid entity, and methionine.

220. The composition of embodiment 1, 4, or 197, wherein the composition comprises isovaleryl-CoA, a R-amino acid entity, a Q-amino acid entity, and D-cysteine.

221. The composition of embodiment 1, 4, or 197, wherein the composition comprises isovaleryl-CoA, a R-amino acid entity, a Q-amino acid entity, and L-cysteine.

222. The composition of embodiment 1, 4, or 197, wherein the composition comprises isovaleryl-CoA, a R-amino acid entity, a Q-amino acid entity, and cysteine.

223. The composition of embodiment 1, 4, or 197, wherein the composition comprises isovaleryl-CoA, a R-amino acid entity, a Q-amino acid entity, and cystine.

224. The composition of embodiment 1, 4, or 197, wherein the composition comprises isovaleryl-CoA, L-arginine, L-glutamine, and an antioxidant or ROS scavenger, e.g., a NAC entity.

225. The composition of embodiment 1, 4, or 197, wherein the composition comprises isovaleryl-CoA, argininosuccinate, glutamate, and an antioxidant or ROS scavenger, e.g., a NAC entity.

226. The composition of embodiment 1, 4, or 197, wherein the composition comprises isovaleryl-CoA, citrulline, D-glutamine, and an antioxidant or ROS scavenger, e.g., a NAC entity.

227. The composition of embodiment 1, 4, or 197, wherein the composition comprises isovaleryl-CoA, aspartate, N-acetyl-glutamine, and an antioxidant or ROS scavenger, e.g., a NAC entity.

228. The composition of embodiment 1, 4, or 197, wherein the composition comprises isovaleryl-CoA, L-arginine, a Q-amino acid entity, and NAC.

229. The composition of embodiment 1, 4, or 197, wherein the composition comprises isovaleryl-CoA, argininosuccinate, a Q-amino acid entity, and serine.

230. The composition of embodiment 1, 4, or 197, wherein the composition comprises isovaleryl-CoA, citrulline, a Q-amino acid entity, and acetylserine.

231. The composition of embodiment 1, 4, or 197, wherein the composition comprises isovaleryl-CoA, aspartate, a Q-amino acid entity, and cystathionine.

232. The composition of embodiment 1, 4, or 197, wherein the composition comprises isovaleryl-CoA, glutamate, a Q-amino acid entity, and glutathione.

233. The composition of embodiment 1, 4, or 197, wherein the composition comprises isovaleryl-CoA, ornithine, a Q-amino acid entity, and homocysteine.

234. The composition of embodiment 1, 4, or 197, wherein the composition comprises isovaleryl-CoA, agmatine, a Q-amino acid entity, and methionine.

235. The composition of embodiment 1, 4, or 197, wherein the composition comprises isovaleryl-CoA, creatine, a Q-amino acid entity, and D-cysteine.

236. The composition of embodiment 1, 4, or 197, wherein the composition comprises isovaleryl-CoA, D-arginine, a Q-amino acid entity, and L-cysteine.

237. The composition of embodiment 1, 4, or 197, wherein the composition comprises isovaleryl-CoA, N-acetyl-arginine, a Q-amino acid entity, and cystine.

238. The composition of embodiment 1, 4, or 197, wherein the composition comprises isovaleryl-CoA, a R-amino acid entity, L-glutamine, and NAC.

239. The composition of embodiment 1, 4, or 197, wherein the composition comprises isovaleryl-CoA, a R-amino acid entity, glutamate, and serine.

240. The composition of embodiment 1, 4, or 197, wherein the composition comprises isovaleryl-CoA, a R-amino acid entity, carbamoyl-P, and acetylserine.

241. The composition of embodiment 1, 4, or 197, wherein the composition comprises isovaleryl-CoA, a R-amino acid entity, N-acetyl-glutamine, and cystathionine.

242. The composition of embodiment 1, 4, or 197, wherein the composition comprises isovaleryl-CoA, a R-amino acid entity, L-glutamine, and glutathione.

243. The composition of embodiment 1, 4, or 197, wherein the composition comprises isovaleryl-CoA, a R-amino acid entity, glutamate, and homocysteine.

244. The composition of embodiment 1, 4, or 197, wherein the composition comprises isovaleryl-CoA, a R-amino acid entity, carbamoyl-P, and methionine.

245. The composition of embodiment 1, 4, or 197, wherein the composition comprises isovaleryl-CoA, a R-amino acid entity, N-acetyl-glutamine, and D-cysteine.

246. The composition of embodiment 1, 4, or 197, wherein the composition comprises isovaleryl-CoA, a R-amino acid entity, L-glutamine, and L-cysteine.

247. The composition of embodiment 1, 4, or 197, wherein the composition comprises isovaleryl-CoA, a R-amino acid entity, a glutamate, and cystine.

248. The composition of embodiment 1, 4, or 197, wherein the composition comprises isovaleryl-CoA, L-arginine, L-glutamine, and NAC.

249. The composition of embodiment 1, 4, or 197, wherein the composition comprises isovaleryl-CoA, argininosuccinate, glutamate, and serine.

250. The composition of embodiment 1, 4, or 197, wherein the composition comprises isovaleryl-CoA, citrulline, carbamoyl-P, and acetylserine.

251. The composition of embodiment 1, 4, or 197, wherein the composition comprises isovaleryl-CoA, aspartate, D-glutamine, and cystathionine.

252. The composition of embodiment 1, 4, or 197, wherein the composition comprises isovaleryl-CoA, N-acetyl-glutamine, L-glutamine, and glutathione.

253. The composition of embodiment 1, 4, or 197, wherein the composition comprises isovaleryl-CoA, ornithine, glutamate, and homocysteine.

254. The composition of embodiment 1, 4, or 197, wherein the composition comprises isovaleryl-CoA, agmatine, carbamoyl-P, and methionine.

255. The composition of embodiment 1, 4, or 197, wherein the composition comprises isovaleryl-CoA, creatine, D-glutamine and D-cysteine.

256. The composition of embodiment 1, 4, or 197, wherein the composition comprises isovaleryl-CoA, D-arginine, a Q-amino acid entity, and L-cysteine.

257. The composition of embodiment 1, 4, or 197, wherein the composition comprises isovaleryl-CoA, N-acetyl-arginine, argininosuccinate, and cystine.

258. The composition of embodiment 1 or 5, wherein the composition comprises D-leucine, a R-amino acid entity, a Q-amino acid entity, and an antioxidant or ROS scavenger, e.g., a NAC entity.

259. The composition of embodiment 1, 5, or 258, wherein the composition comprises D-leucine, L-arginine, a Q-amino acid entity, and an antioxidant or ROS scavenger, e.g., a NAC entity.

260. The composition of embodiment 1, 5, or 258, wherein the composition comprises D-leucine, argininosuccinate, a Q-amino acid entity, and an antioxidant or ROS scavenger, e.g., a NAC entity.

261. The composition of embodiment 1, 5, or 258, wherein the composition comprises D-leucine, citrulline, a Q-amino acid entity, and an antioxidant or ROS scavenger, e.g., a NAC entity.

262. The composition of embodiment 1, 5, or 258, wherein the composition comprises D-leucine, aspartate, a Q-amino acid entity, and an antioxidant or ROS scavenger, e.g., a NAC entity.

263. The composition of embodiment 1, 5, or 258, wherein the composition comprises D-leucine, glutamate, a Q-amino acid entity, and an antioxidant or ROS scavenger, e.g., a NAC entity.

264. The composition of embodiment 1, 5, or 258, wherein composition comprises D-leucine, ornithine, a Q-amino acid entity, and an antioxidant or ROS scavenger, e.g., a NAC entity.

265. The composition of embodiment 1, 5, or 258, wherein the composition comprises D-leucine, agmatine, a Q-amino acid entity, and an antioxidant or ROS scavenger, e.g., a NAC entity.

266. The composition of embodiment 1, 5, or 258, wherein the composition comprises D-leucine, creatine, a Q-amino acid entity, and an antioxidant or ROS scavenger, e.g., a NAC entity.

267. The composition of embodiment 1, 5, or 258, wherein the composition comprises D-leucine, D-arginine, a Q-amino acid entity, and an antioxidant or ROS scavenger, e.g., a NAC entity.

268. The composition of embodiment 1, 5, or 258, wherein the composition comprises D-leucine, N-acetyl-arginine, a Q-amino acid entity, and an antioxidant or ROS scavenger, e.g., a NAC entity.

269. The composition of embodiment 1, 5, or 258, wherein the composition comprises D-leucine, a R-amino acid entity, L-glutamine, and an antioxidant or ROS scavenger, e.g., a NAC entity.

270. The composition of embodiment 1, 5, or 258, wherein the composition comprises D-leucine, a R-amino acid entity, glutamate, and an antioxidant or ROS scavenger, e.g., a NAC entity.

271. The composition of embodiment 1, 5, or 258, wherein the composition comprises D-leucine, a R-amino acid entity, carbamoyl-P, and an antioxidant or ROS scavenger, e.g., a NAC entity.

272. The composition of embodiment 1, 5, or 258, wherein the composition comprises D-leucine, a R-amino acid entity, D-glutamine, and an antioxidant or ROS scavenger, e.g., a NAC entity.

273. The composition of embodiment 1, 5, or 258, wherein the composition comprises D-leucine, a R-amino acid entity, N-acetyl-glutamine, and an antioxidant or ROS scavenger, e.g., a NAC entity.

274. The composition of embodiment 1, 5, or 258, wherein the composition comprises D-leucine, a R-amino acid entity, a Q-amino acid entity, and NAC.

275. The composition of embodiment 1, 5, or 258, wherein the composition comprises D-leucine, a R-amino acid entity, a Q-amino acid entity, and serine.

276. The composition of embodiment 1, 5, or 258, wherein the composition comprises D-leucine, a R-amino acid entity, a Q-amino acid entity, and acetylserine.

277. The composition of embodiment 1, 5, or 258, wherein the composition comprises D-leucine, a R-amino acid entity, a Q-amino acid entity, and cystathionine.

278. The composition of embodiment 1, 5, or 258, wherein the composition comprises D-leucine, a R-amino acid entity, a Q-amino acid entity, and glutathione.

279. The composition of embodiment 1, 5, or 258, wherein the composition comprises D-leucine, a R-amino acid entity, a Q-amino acid entity, and homocysteine.

280. The composition of embodiment 1, 5, or 258, wherein the composition comprises D-leucine, a R-amino acid entity, a Q-amino acid entity, and methionine.

281. The composition of embodiment 1, 5, or 258, wherein the composition comprises D-leucine, a R-amino acid entity, a Q-amino acid entity, and D-cysteine.

282. The composition of embodiment 1, 5, or 258, wherein the composition comprises D-leucine, a R-amino acid entity, a Q-amino acid entity, and L-cysteine.

283. The composition of embodiment 1, 5, or 258, wherein the composition comprises D-leucine, a R-amino acid entity, a Q-amino acid entity, and cysteine.

284. The composition of embodiment 1, 5, or 258, wherein the composition comprises D-leucine, a R-amino acid entity, a Q-amino acid entity, and cystine.

285. The composition of embodiment 1, 5, or 258, wherein the composition comprises D-leucine, L-arginine, L-glutamine, and an antioxidant or ROS scavenger, e.g., a NAC entity.

286. The composition of embodiment 1, 5, or 258, wherein the composition comprises D-leucine, argininosuccinate, glutamate, and an antioxidant or ROS scavenger, e.g., a NAC entity.

287. The composition of embodiment 1, 5, or 258, wherein the composition comprises D-leucine, citrulline, D-glutamine, and an antioxidant or ROS scavenger, e.g., a NAC entity.

288. The composition of embodiment 1, 5, or 258, wherein the composition comprises D-leucine, aspartate, N-acetyl-glutamine, and an antioxidant or ROS scavenger, e.g., a NAC entity.

289. The composition of embodiment 1, 5, or 258, wherein the composition comprises D-leucine, L-arginine, a Q-amino acid entity, and NAC.

290. The composition of embodiment 1, 5, or 258, wherein the composition comprises D-leucine, argininosuccinate, a Q-amino acid entity, and serine.

291. The composition of embodiment 1, 5, or 258, wherein the composition comprises D-leucine, citrulline, a Q-amino acid entity, and acetylserine.

292. The composition of embodiment 1, 5, or 258, wherein the composition comprises D-leucine, aspartate, a Q-amino acid entity, and cystathionine.

293. The composition of embodiment 1, 5, or 258, wherein the composition comprises D-leucine, glutamate, a Q-amino acid entity, and glutathione.

294. The composition of embodiment 1, 5, or 258, wherein the composition comprises D-leucine, ornithine, a Q-amino acid entity, and homocysteine.

295. The composition of embodiment 1, 5, or 258, wherein the composition comprises D-leucine, agmatine, a Q-amino acid entity, and methionine.

296. The composition of embodiment 1, 5, or 258, wherein the composition comprises D-leucine, creatine, a Q-amino acid entity, and D-cysteine.

297. The composition of embodiment 1, 5, or 258, wherein the composition comprises D-leucine, D-arginine, a Q-amino acid entity, and L-cysteine.

298. The composition of embodiment 1, 5, or 258, wherein the composition comprises D-leucine, N-acetyl-arginine, a Q-amino acid entity, and cystine.

299. The composition of embodiment 1, 5, or 258, wherein the composition comprises D-leucine, a R-amino acid entity, L-glutamine, and NAC.

300. The composition of embodiment 1, 5, or 258, wherein the composition comprises D-leucine, a R-amino acid entity, glutamate, and serine.

301. The composition of embodiment 1, 5, or 258, wherein the composition comprises D-leucine, a R-amino acid entity, carbamoyl-P, and acetylserine.

302. The composition of embodiment 1, 5, or 258, wherein the composition comprises D-leucine, a R-amino acid entity, N-acetyl-glutamine, and cystathionine.

303. The composition of embodiment 1, 5, or 258, wherein the composition comprises D-leucine, a R-amino acid entity, L-glutamine, and glutathione.

304. The composition of embodiment 1, 5, or 258, wherein the composition comprises D-leucine, a R-amino acid entity, glutamate, and homocysteine.

305. The composition of embodiment 1, 5, or 258, wherein the composition comprises D-leucine, a R-amino acid entity, carbamoyl-P, and methionine.

306. The composition of embodiment 1, 5, or 258, wherein the composition comprises D-leucine, a R-amino acid entity, N-acetyl-glutamine, and D-cysteine.

307. The composition of embodiment 1, 5, or 258, wherein the composition comprises D-leucine, a R-amino acid entity, L-glutamine, and L-cysteine.

308. The composition of embodiment 1, 5, or 258, wherein the composition comprises D-leucine, a R-amino acid entity, a glutamate, and cystine.

309. The composition of embodiment 1, 5, or 258, wherein the composition comprises D-leucine, L-arginine, L-glutamine, and NAC.

310. The composition of embodiment 1, 5, or 258, wherein the composition comprises D-leucine, argininosuccinate, glutamate, and serine.

311. The composition of embodiment 1, 5, or 258, wherein the composition comprises D-leucine, citrulline, carbamoyl-P, and acetylserine.

312. The composition of embodiment 1, 5, or 258, wherein the composition comprises D-leucine, aspartate, D-glutamine, and cystathionine.

313. The composition of embodiment 1, 5, or 258, wherein the composition comprises D-leucine, N-acetyl-glutamine, L-glutamine, and glutathione.

314. The composition of embodiment 1, 5, or 258, wherein the composition comprises D-leucine, ornithine, glutamate, and homocysteine.

315. The composition of embodiment 1, 5, or 258, wherein the composition comprises D-leucine, agmatine, carbamoyl-P, and methionine.

316. The composition of embodiment 1, 5, or 258, wherein the composition comprises D-leucine, creatine, D-glutamine and D-cysteine.

317. The composition of embodiment 1, 5, or 258, wherein the composition comprises D-leucine, D-arginine, a Q-amino acid entity, and L-cysteine.

318. The composition of embodiment 1, 5, or 258, wherein the composition comprises D-leucine, N-acetyl-arginine, argininosuccinate, and cystine.

319. The composition of embodiment 1 or 5, wherein the composition comprises N-acetyl-leucine, a R-amino acid entity, a Q-amino acid entity, and an antioxidant or ROS scavenger, e.g., a NAC entity.

320. The composition of embodiment 1, 5, or 319, wherein the composition comprises N-acetyl-leucine, L-arginine, a Q-amino acid entity, and an antioxidant or ROS scavenger, e.g., a NAC entity.

321. The composition of embodiment 1, 5, or 319, wherein the composition comprises N-acetyl-leucine, argininosuccinate, a Q-amino acid entity, and an antioxidant or ROS scavenger, e.g., a NAC entity.

322. The composition of embodiment 1, 5, or 319, wherein the composition comprises N-acetyl-leucine, citrulline, a Q-amino acid entity, and an antioxidant or ROS scavenger, e.g., a NAC entity.

323. The composition of embodiment 1, 5, or 319, wherein the composition comprises N-acetyl-leucine, aspartate, a Q-amino acid entity, and an antioxidant or ROS scavenger, e.g., a NAC entity.

324. The composition of embodiment 1, 5, or 319, wherein the composition comprises N-acetyl-leucine, glutamate, a Q-amino acid entity, and an antioxidant or ROS scavenger, e.g., a NAC entity.

325. The composition of embodiment 1, 5, or 319, wherein the composition comprises N-acetyl-leucine, ornithine, a Q-amino acid entity, and an antioxidant or ROS scavenger, e.g., a NAC entity.

326. The composition of embodiment 1, 5, or 319, wherein the composition comprises N-acetyl-leucine, agmatine, a Q-amino acid entity, and an antioxidant or ROS scavenger, e.g., a NAC entity.

327. The composition of embodiment 1, 5, or 319, wherein the composition comprises N-acetyl-leucine, creatine, a Q-amino acid entity, and an antioxidant or ROS scavenger, e.g., a NAC entity.

328. The composition of embodiment 1, 5, or 319, wherein the composition comprises N-acetyl-leucine, D-arginine, a Q-amino acid entity, and an antioxidant or ROS scavenger, e.g., a NAC entity.

329. The composition of embodiment 1, 5, or 319, wherein the composition comprises N-acetyl-leucine, N-acetyl-arginine, a Q-amino acid entity, and an antioxidant or ROS scavenger, e.g., a NAC entity.

330. The composition of embodiment 1, 5, or 319, wherein the composition comprises N-acetyl-leucine, a R-amino acid entity, L-glutamine, and an antioxidant or ROS scavenger, e.g., a NAC entity.

331. The composition of embodiment 1, 5, or 319, wherein the composition comprises N-acetyl-leucine, a R-amino acid entity, glutamate, and an antioxidant or ROS scavenger, e.g., a NAC entity.

332. The composition of embodiment 1, 5, or 319, wherein the composition comprises N-acetyl-leucine, a R-amino acid entity, carbamoyl-P, and an antioxidant or ROS scavenger, e.g., a NAC entity.

333. The composition of embodiment 1, 5, or 319, wherein the composition comprises N-acetyl-leucine, a R-amino acid entity, D-glutamine, and an antioxidant or ROS scavenger, e.g., a NAC entity.

334. The composition of embodiment 1, 5, or 319, wherein the composition comprises N-acetyl-leucine, a R-amino acid entity, N-acetyl-glutamine, and an antioxidant or ROS scavenger, e.g., a NAC entity.

335. The composition of embodiment 1, 5, or 319, wherein the composition comprises N-acetyl-leucine, a R-amino acid entity, a Q-amino acid entity, and NAC.

336. The composition of embodiment 1, 5, or 319, wherein the composition comprises N-acetyl-leucine, a R-amino acid entity, a Q-amino acid entity, and serine.

337. The composition of embodiment 1, 5, or 319, wherein the composition comprises N-acetyl-leucine, a R-amino acid entity, a Q-amino acid entity, and acetylserine.

338. The composition of embodiment 1, 5, or 319, wherein the composition comprises N-acetyl-leucine, a R-amino acid entity, a Q-amino acid entity, and cystathionine.

339. The composition of embodiment 1, 5, or 319, wherein the composition comprises N-acetyl-leucine, a R-amino acid entity, a Q-amino acid entity, and glutathione.

340. The composition of embodiment 1, 5, or 319, wherein the composition comprises N-acetyl-leucine, a R-amino acid entity, a Q-amino acid entity, and homocysteine.

341. The composition of embodiment 1, 5, or 319, wherein the composition comprises N-acetyl-leucine, a R-amino acid entity, a Q-amino acid entity, and methionine.

342. The composition of embodiment 1, 5, or 319, wherein the composition comprises N-acetyl-leucine, a R-amino acid entity, a Q-amino acid entity, and D-cysteine.

343. The composition of embodiment 1, 5, or 319, wherein the composition comprises N-acetyl-leucine, a R-amino acid entity, a Q-amino acid entity, and L-cysteine.

344. The composition of embodiment 1, 5, or 319, wherein the composition comprises N-acetyl-leucine, a R-amino acid entity, a Q-amino acid entity, and cysteine.

345. The composition of embodiment 1, 5, or 319, wherein the composition comprises N-acetyl-leucine, a R-amino acid entity, a Q-amino acid entity, and cystine.

346. The composition of embodiment 1, 5, or 319, wherein the composition comprises N-acetyl-leucine, L-arginine, L-glutamine, and an antioxidant or ROS scavenger, e.g., a NAC entity.

347. The composition of embodiment 1, 5, or 319, wherein the composition comprises N-acetyl-leucine, argininosuccinate, glutamate, and an antioxidant or ROS scavenger, e.g., a NAC entity.

348. The composition of embodiment 1, 5, or 319, wherein the composition comprises N-acetyl-leucine, citrulline, D-glutamine, and an antioxidant or ROS scavenger, e.g., a NAC entity.

349. The composition of embodiment 1, 5, or 319, wherein the composition comprises N-acetyl-leucine, aspartate, N-acetyl-glutamine, and an antioxidant or ROS scavenger, e.g., a NAC entity.

350. The composition of embodiment 1, 5, or 319, wherein the composition comprises N-acetyl-leucine, L-arginine, a Q-amino acid entity, and NAC.

351. The composition of embodiment 1, 5, or 319, wherein the composition comprises N-acetyl-leucine, argininosuccinate, a Q-amino acid entity, and serine.

352. The composition of embodiment 1, 5, or 319, wherein the composition comprises N-acetyl-leucine, citrulline, a Q-amino acid entity, and acetylserine.

353. The composition of embodiment 1, 5, or 319, wherein the composition comprises N-acetyl-leucine, aspartate, a Q-amino acid entity, and cystathionine.

354. The composition of embodiment 1, 5, or 319, wherein the composition comprises N-acetyl-leucine, glutamate, a Q-amino acid entity, and glutathione.

355. The composition of embodiment 1, 5, or 319, wherein the composition comprises N-acetyl-leucine, ornithine, a Q-amino acid entity, and homocysteine.

356. The composition of embodiment 1, 5, or 319, wherein the composition comprises N-acetyl-leucine, agmatine, a Q-amino acid entity, and methionine.

357. The composition of embodiment 1, 5, or 319, wherein the composition comprises N-acetyl-leucine, creatine, a Q-amino acid entity, and D-cysteine.

358. The composition of embodiment 1, 5, or 319, wherein the composition comprises N-acetyl-leucine, D-arginine, a Q-amino acid entity, and L-cysteine.

359. The composition of embodiment 1, 5, or 319, wherein the composition comprises N-acetyl-leucine, N-acetyl-arginine, a Q-amino acid entity, and cystine.

360. The composition of embodiment 1, 5, or 319, wherein the composition comprises N-acetyl-leucine, a R-amino acid entity, L-glutamine, and NAC.

361. The composition of embodiment 1, 5, or 319, wherein the composition comprises N-acetyl-leucine, a R-amino acid entity, glutamate, and serine.

362. The composition of embodiment 1, 5, or 319, wherein the composition comprises N-acetyl-leucine, a R-amino acid entity, carbamoyl-P, and acetylserine.

363. The composition of embodiment 1, 5, or 319, wherein the composition comprises N-acetyl-leucine, a R-amino acid entity, N-acetyl-glutamine, and cystathionine.

364. The composition of embodiment 1, 5, or 319, wherein the composition comprises N-acetyl-leucine, a R-amino acid entity, L-glutamine, and glutathione.

365. The composition of embodiment 1, 5, or 319, wherein the composition comprises N-acetyl-leucine, a R-amino acid entity, glutamate, and homocysteine.

366. The composition of embodiment 1, 5, or 319, wherein the composition comprises N-acetyl-leucine, a R-amino acid entity, carbamoyl-P, and methionine.

367. The composition of embodiment 1, 5, or 319, wherein the composition comprises N-acetyl-leucine, a R-amino acid entity, N-acetyl-glutamine, and D-cysteine.

368. The composition of embodiment 1, 5, or 319, wherein the composition comprises N-acetyl-leucine, a R-amino acid entity, L-glutamine, and L-cysteine.

369. The composition of embodiment 1, 5, or 319, wherein the composition comprises N-acetyl-leucine, a R-amino acid entity, a glutamate, and cystine.

370. The composition of embodiment 1, 5, or 319, wherein the composition comprises N-acetyl-leucine, L-arginine, L-glutamine, and NAC.

371. The composition of embodiment 1, 5, or 319, wherein the composition comprises N-acetyl-leucine, argininosuccinate, glutamate, and serine.

372. The composition of embodiment 1, 5, or 319, wherein the composition comprises N-acetyl-leucine, citrulline, carbamoyl-P, and acetylserine.

373. The composition of embodiment 1, 5, or 319, wherein the composition comprises N-acetyl-leucine, aspartate, D-glutamine, and cystathionine.

374. The composition of embodiment 1, 5, or 319, wherein the composition comprises N-acetyl-leucine, N-acetyl-glutamine, L-glutamine, and glutathione.

375. The composition of embodiment 1, 5, or 319, wherein the composition comprises N-acetyl-leucine, ornithine, glutamate, and homocysteine.

376. The composition of embodiment 1, 5, or 319, wherein the composition comprises N-acetyl-leucine, agmatine, carbamoyl-P, and methionine.

377. The composition of embodiment 1, 5, or 319, wherein the composition comprises N-acetyl-leucine, creatine, D-glutamine and D-cysteine.

378. The composition of embodiment 1, 5, or 319, wherein the composition comprises N-acetyl-leucine, D-arginine, a Q-amino acid entity, and L-cysteine.

379. The composition of embodiment 1, 5, or 319, wherein the composition comprises N-acetyl-leucine, N-acetyl-arginine, argininosuccinate, and cystine.

380. The composition of embodiment 1 or 2, wherein the composition comprises a L-amino acid entity, L-arginine, a Q-amino acid entity, and an antioxidant or ROS scavenger, e.g., a NAC entity.

381. The composition of embodiment 1 or 2, wherein the composition comprises a L-amino acid entity, argininosuccinate, a Q-amino acid entity, and an antioxidant or ROS scavenger, e.g., a NAC entity.

382. The composition of embodiment 1, 3, or 4, wherein the composition comprises a L-amino acid entity, citrulline, a Q-amino acid entity, and an antioxidant or ROS scavenger, e.g., a NAC entity.

383. The composition of embodiment 1 or 3, wherein the composition comprises a L-amino acid entity, aspartate, a Q-amino acid entity, and an antioxidant or ROS scavenger, e.g., a NAC entity.

384. The composition of embodiment 1 or 3, wherein the composition comprises a L-amino acid entity, glutamate, a Q-amino acid entity, and an antioxidant or ROS scavenger, e.g., a NAC entity.

385. The composition of embodiment 1 or 4, wherein the composition comprises a L-amino acid entity, ornithine, a Q-amino acid entity, and an antioxidant or ROS scavenger, e.g., a NAC entity.

386. The composition of embodiment 1 or 4, wherein the composition comprises a L-amino acid entity, agmatine, a Q-amino acid entity, and an antioxidant or ROS scavenger, e.g., a NAC entity.

387. The composition of embodiment 1 or 4, wherein the composition comprises a L-amino acid entity, creatine, a Q-amino acid entity, and an antioxidant or ROS scavenger, e.g., a NAC entity.

388. The composition of embodiment 1 or 5, wherein the composition comprises a L-amino acid entity, D-arginine, a Q-amino acid entity, and an antioxidant or ROS scavenger, e.g., a NAC entity.

389. The composition of embodiment 1 or 5, wherein the composition comprises a L-amino acid entity, N-acetyl-arginine, a Q-amino acid entity, and an antioxidant or ROS scavenger, e.g., a NAC entity.

390. The composition of embodiment 1, 3, or 384, wherein the composition comprises L-leucine, glutamate, L-glutamine, and an antioxidant or ROS scavenger, e.g., a NAC entity.

391. The composition of embodiment 1, 4, or 385, wherein the composition comprises L-leucine, ornithine, L-glutamine, and an antioxidant or ROS scavenger, e.g., a NAC entity.

392. The composition of embodiment 1, 4, or 386, wherein the composition comprises a L-amino acid entity, agmatine, L-glutamine, and an antioxidant or ROS scavenger, e.g., a NAC entity.

393. The composition of embodiment 1, 4, or 387, wherein the composition comprises a L-amino acid entity, creatine, L-glutamine, and an antioxidant or ROS scavenger, e.g., a NAC entity.

394. The composition of embodiment 1, 4, or 388, wherein the composition comprises a L-amino acid entity, D-arginine, L-glutamine, and an antioxidant or ROS scavenger, e.g., a NAC entity.

395. The composition of embodiment 1, 4, or 389, wherein the composition comprises a L-amino acid entity, D-arginine, N-acetyl-arginine, and an antioxidant or ROS scavenger, e.g., a NAC entity.

396. The composition of embodiment 1 or 380, wherein the composition comprises a L-amino acid entity, L-arginine, L-glutamine, and an antioxidant or ROS scavenger, e.g., a NAC entity.

397. The composition of embodiment 1, 2, or 381, wherein the composition comprises a L-amino acid entity, argininosuccinate, glutamate, and an antioxidant or ROS scavenger, e.g., a NAC entity.

398. The composition of embodiment 1, 3, 4, or 382, wherein the composition comprises a L-amino acid entity, citrulline, carbamoyl-P, and an antioxidant or ROS scavenger, e.g., a NAC entity.

399. The composition of embodiment 1, 3, or 383, wherein the composition comprises a L-amino acid entity, aspartate, glutamate, and an antioxidant or ROS scavenger, e.g., a NAC entity.

400. The composition of embodiment 1, 3, or 384, wherein the composition comprises a L-amino acid entity, glutamate, D-glutamine, and an antioxidant or ROS scavenger, e.g., a NAC entity.

401. The composition of embodiment 1, 4, or 385, wherein the composition comprises a L-amino acid entity, ornithine, N-acetyl-glutamine, and an antioxidant or ROS scavenger, e.g., a NAC entity.

402. The composition of embodiment 1, 4, or 386, wherein the composition comprises a L-amino acid entity, agmatine, L-glutamine, and an antioxidant or ROS scavenger, e.g., a NAC entity.

403. The composition of embodiment 1, 4, or 387, wherein the composition comprises a L-amino acid entity, creatine, glutamate, and an antioxidant or ROS scavenger, e.g., a NAC entity.

404. The composition of embodiment 1, 5, or 388, wherein the composition comprises a L-amino acid entity, D-arginine, carbamoyl-P, and an antioxidant or ROS scavenger, e.g., a NAC entity.

405. The composition of embodiment 1, 5, or 389, wherein the composition comprises a L-amino acid entity, N-acetyl-arginine, glutamate, and an antioxidant or ROS scavenger, e.g., a NAC entity.

406. The composition of embodiment 1, 380, or 445, wherein the composition comprises a L-amino acid entity, L-arginine, L-glutamine, and NAC.

407. The composition of embodiment 1, 2, 381, or 446, wherein the composition comprises a L-amino acid entity, argininosuccinate, glutamate, and serine.

408. The composition of embodiment 1, 3, 4, 382, or 447, wherein the composition comprises a L-amino acid entity, citrulline, carbamoyl-P, and acetylserine.

409. The composition of embodiment 1, 3, 383, or 448, wherein the composition comprises a L-amino acid entity, aspartate, glutamate, and cystathionine.

410. The composition of embodiment 1, 3, 384, or 449, wherein the composition comprises a L-amino acid entity, glutamate, D-glutamine, and glutathione.

411. The composition of embodiment 1, 4, 385, or 448, wherein the composition comprises a L-amino acid entity, ornithine, N-acetyl-glutamine, and cystathionine.

412. The composition of embodiment 1, 4, 386, or 450, wherein the composition comprises a L-amino acid entity, agmatine, L-glutamine, and homocysteine.

413. The composition of embodiment 1, 4, 387, or 451, wherein the composition comprises a L-amino acid entity, creatine, glutamate, and methionine.

414. The composition of embodiment 1, 5, 388, or 454, wherein the composition comprises a L-amino acid entity, D-arginine, carbamoyl-P, and D-cysteine.

415. The composition of embodiment 1, 5, 389, or 453, wherein the composition comprises a L-amino acid entity, N-acetyl-arginine, glutamate, and L-cysteine.

416. The composition of embodiment 1 380, or 454, wherein the composition comprises a L-amino acid entity, L-arginine, L-glutamine, and cystine.

417. The composition of embodiment 1, 6, or 445, wherein the composition comprises a L-amino acid entity, L-arginine, a Q-amino acid, and NAC.

418. The composition of embodiment 1, 2, or 446, wherein the composition comprises a L-amino acid entity, argininosuccinate, a Q-amino acid, and serine.

419. The composition of embodiment 1, 3, or 447, wherein the composition comprises a L-amino acid entity, citrulline, a Q-amino acid, and acetylserine.

420. The composition of embodiment 1, 4, or 448, wherein the composition comprises a L-amino acid entity, aspartate, a Q-amino acid, and cystathionine.

421. The composition of embodiment 1, 3, or 449, wherein the composition comprises a L-amino acid entity, glutamate, a Q-amino acid, and glutathione.

422. The composition of embodiment 1, 4, or 448, wherein the composition comprises a L-amino acid entity, ornithine, a Q-amino acid, and cystathionine.

423. The composition of embodiment 1, 4, or 450, wherein the composition comprises a L-amino acid entity, agmatine, a Q-amino acid, and homocysteine.

424. The composition of embodiment 1, 4, or 451, wherein the composition comprises a L-amino acid entity, creatine, a Q-amino acid, and methionine.

425. The composition of embodiment 1, 5, or 452, wherein the composition comprises a L-amino acid entity, D-arginine, a Q-amino acid, and D-cysteine.

426. The composition of embodiment 1, 5, or 453, wherein the composition comprises a L-amino acid entity, N-acetyl-arginine, a Q-amino acid, and L-cysteine.

427. The composition of embodiment 1, 5, or 454, wherein the composition comprises a L-amino acid entity, L-arginine, a Q-amino acid, and cystine.

428. The composition of embodiment 1 or 2, wherein the composition comprises a L-amino acid entity, a R-amino acid entity, L-glutamine, and an antioxidant or ROS scavenger, e.g., a NAC entity.

429. The composition of embodiment 1, 3, or 4, wherein the composition comprises a L-amino acid entity, a R-amino acid entity, glutamate, and an antioxidant or ROS scavenger, e.g., a NAC entity.

430. The composition of embodiment 1 or 4, wherein the composition comprises a L-amino acid entity, a R-amino acid entity, carbamoyl-P, and an antioxidant or ROS scavenger, e.g., a NAC entity.

431. The composition of embodiment 1 or 5, wherein the composition comprises a L-amino acid entity, a R-amino acid entity, D-glutamine, and an antioxidant or ROS scavenger, e.g., a NAC entity.

432. The composition of embodiment 1 or 5, wherein the composition comprises a L-amino acid entity, a R-amino acid entity, N-acetyl-glutamine, and an antioxidant or ROS scavenger, e.g., a NAC entity.

433. The composition of embodiment 1, 5, or 431, wherein the composition comprises a L-leucine, a R-amino acid entity, D-glutamine, and an antioxidant or ROS scavenger, e.g., a NAC entity.

434. The composition of embodiment 1, 4 or 430, wherein the composition comprises a L-leucine, L-arginine, carbamoyl-P, and an antioxidant or ROS scavenger, e.g., a NAC entity.

435. The composition of embodiment 1, 2, 428, or 445, wherein the composition comprises a L-amino acid entity, a R-amino acid entity, L-glutamine, and NAC.

436. The composition of embodiment 1, 3, 4, 429, or 446, wherein the composition comprises a L-amino acid entity, a R-amino acid entity, glutamate, and serine.

437. The composition of embodiment 1, 4, 430, or 447, wherein the composition comprises a L-amino acid entity, a R-amino acid entity, carbamoyl-P, and acetylserine.

438. The composition of embodiment 1, 5, 431, or 448, wherein the composition comprises a L-amino acid entity, a R-amino acid entity, D-glutamine, and cystathionine.

439. The composition of embodiment 1, 5, 432, or 449, wherein the composition comprises a L-amino acid entity, a R-amino acid entity, N-acetyl-glutamine, and glutathione.

440. The composition of embodiment 1, 2, 428, or 450, wherein the composition comprises a L-amino acid entity, a R-amino acid entity, L-glutamine, and homocysteine.

441. The composition of embodiment 1, 3, 4, 429, or 451, wherein the composition comprises a L-amino acid entity, a R-amino acid entity, glutamate, and methionine.

442. The composition of embodiment 1, 4, 430, or 452, wherein the composition comprises a L-amino acid entity, a R-amino acid entity, carbamoyl-P, and D-cysteine

443. The composition of embodiment 1, 5, 431, or 453, wherein the composition comprises a L-amino acid entity, a R-amino acid entity, D-glutamine, and L-cysteine.

444. The composition of embodiment 1, 5, 432, or 454, wherein the composition comprises a L-amino acid entity, a R-amino acid entity, N-acetyl-glutamine, and cystine.

445. The composition of embodiment 1 or 5, wherein the composition comprises a L-amino acid entity, a R-amino acid entity, a Q-amino acid entity, and NAC.

446. The composition of embodiment 1 or 3, wherein the composition comprises a L-amino acid entity, a R-amino acid entity, a Q-amino acid entity, and serine.

447. The composition of embodiment 1 or 3, wherein the composition comprises a L-amino acid entity, a R-amino acid entity, a Q-amino acid entity, and acetylserine.

448. The composition of embodiment 1 or 3, wherein the composition comprises a L-amino acid entity, a R-amino acid entity, a Q-amino acid entity, and cystathionine.

449. The composition of embodiment 1 or 4, wherein the composition comprises a L-amino acid entity, a R-amino acid entity, a Q-amino acid entity, and glutathione.

450. The composition of embodiment 1 or 4, wherein the composition comprises a L-amino acid entity, a R-amino acid entity, a Q-amino acid entity, and homocysteine.

451. The composition of embodiment 1 or 4, wherein the composition comprises a L-amino acid entity, a R-amino acid entity, a Q-amino acid entity, and methionine.

452. The composition of embodiment 1 or 5, wherein the composition comprises a L-amino acid entity, a R-amino acid entity, a Q-amino acid entity, and D-cysteine.

453. The composition of embodiment 1 or 5, wherein the composition comprises a L-amino acid entity, a R-amino acid entity, a Q-amino acid entity, and L-cysteine.

454. The composition of embodiment 1 or 5, wherein the composition comprises a L-amino acid entity, a R-amino acid entity, a Q-amino acid entity, and cystine.

455. The composition of embodiment 1 or 2, wherein the composition comprises a L-amino acid, ornithine, a Q-amino acid entity, and an antioxidant or ROS scavenger, e.g., a NAC entity.

456. The composition of embodiment 1 or 455, wherein the composition comprises L-leucine, ornithine, 1-glutamine, and NAC.

457. The composition of embodiment 1 or 455, wherein the composition comprises HMB, ornithine, 1-glutamine, and NAC.

458. The composition of any of the foregoing embodiments, wherein the composition comprises L-leucine or a leucine metabolite (e.g., HMB), 1-arginine or an L-arginine metabolite (e.g., creatine), 1-glutamine, and NAC or a NAC metabolite, e.g., glutathione.

459. The composition of any of the foregoing embodiments, wherein the composition comprises L-leucine or a leucine metabolite (e.g., HMB), L-arginine or an L-arginine metabolite (e.g., creatine), L-glutamine, and NAC or a NAC metabolite, e.g., glutathione.

460. The composition of any of the previous embodiments, further comprising an isoleucine (I)-amino acid entity.

461. The composition of embodiment 460, wherein the I-amino acid entity is an amino acid.

462. The composition of embodiment 460 or 461, wherein the amino acid entity is L-isoleucine.

463. The composition of embodiment 460, wherein the I-amino acid entity is an amino acid precursor.

464. The composition of embodiment 460 or 463, wherein the I-amino acid entity is 2-oxo-3-methyl-valerate.

465. The composition of embodiment 460 or 463, wherein the I-amino acid entity is threonine.

466. The composition of embodiment 460, wherein the I-amino acid entity is an amino acid metabolite.

467. The composition of embodiment 460 or 466, wherein the I-amino acid entity is 2-oxo-3-methyl-valerate

468. The composition of embodiment 460 or 466, wherein the I-amino acid entity is methylbutyrl-CoA.

469. The composition of embodiment 460, wherein the I-amino acid entity is an amino acid derivative.

470. The composition of embodiment 460 or 469, wherein the I-amino acid entity is D-isoleucine.

471. The composition of embodiment 460 or 469, wherein the I-amino acid entity is N-acetyl-isoleucine.

472. The composition of any of the previous embodiments, further comprising a valine (V)-amino acid entity.

473. The composition of embodiment 472, wherein the V-amino acid entity is an amino acid.

474. The composition of embodiment 472 or 473, wherein the V-amino acid entity is L-valine.

475. The composition of embodiment 472, wherein the V-amino acid entity is an amino acid precursor.

476. The composition of embodiment 472 or 475, wherein the V-amino acid entity is 2-oxo-valerate.

477. The composition of embodiment 472, wherein the V-amino acid entity is an amino acid metabolite.

478. The composition of embodiment 472 or 477, wherein the V-amino acid entity is isobutryl-CoA.

479. The composition of embodiment 472 or 477, wherein the V-amino acid entity is 3-HIB-CoA.

480. The composition of embodiment 472 or 477, wherein the V-amino acid entity is 3-HIB.

481. The composition of embodiment 472, wherein the V-amino acid entity is an amino acid derivative.

482. The composition of embodiment 472 or 481, wherein the V-amino acid entity is D-valine.

483. The composition of embodiment 472 or 481, wherein the V-amino acid entity is N-acetyl-valine.

484. The composition of any of the preceding embodiments, wherein the composition further comprises one or more essential amino acid (EAA)-entities.

485. The composition of embodiment 484, wherein the EAA-entities are chosen from one, two, three, or four of a H-amino acid-entity, a K-amino acid-entity, a F-amino acid-entity, and a T-amino acid-entity.

486. The composition of embodiment 485, wherein the H-amino acid-entity is present, e.g., the H-amino acid-entity is present in an amount of at least 0.5 wt. %, at least 0.6 wt. %, at least 0.7 wt. %, at least 0.8 wt. %, at least 0.9 wt. %, at least 1.0 wt. %, at least 1.1 wt. %, at least 1.2 wt. %, at least 1.3 wt. % or at least 1.4 wt. % of the composition.

487. The composition of embodiment 486, wherein the H-amino acid-entity is selected from the group consisting of a precursor, a metabolite, and a derivative.

488. The composition of embodiment 486 or 487, wherein the H-amino acid-entity is selected from the group consisting of L-histidine, histidinol, histidinal, ribose-5-phosphate, carnosine, histamine, urocanate, D-histidine, and N-acetyl-histidine.

489. The composition of any of embodiments 485-488, wherein the K-amino acid-entity is present, e.g., the K-amino acid-entity is present in amount of at least 2 wt. %, at least 3 wt. %, at least 4 wt. %, at least 5 wt. %, or at least 6 wt. % of the composition.

490. The composition of embodiment 489, wherein the K-amino acid-entity is selected from the group consisting of a precursor, a metabolite, and a derivative.

491. The composition of embodiment 488 or 489, wherein the K-amino acid-entity is selected from the group consisting of L-lysine, diaminopimelate, aspartate, trimethyllysine, carnitine, saccharopine, D-lysine, and N-acetyl-lysine.

492. The composition of any of embodiments 485-491, wherein the F-amino acid-entity is present, e.g., the F-amino acid-entity is present in an amount of at least 0.5 wt. %, at least 0.6 wt. %, at least 0.7 wt. %, at least 0.8 wt. %, at least 0.9 wt. %, at least 1.0 wt. %, at least 1.1 wt. %, at least 1.2 wt. %, at least 1.3 wt. % or at least 1.4 wt. % of the composition.

493. The composition of embodiment 492, wherein the F-amino acid-entity is selected from the group consisting of a precursor, a metabolite, and a derivative.

494. The composition of embodiment 492 or 493, wherein the F-amino acid-entity is selected from the group consisting of L-phenylalanine, phenylpyruvate, tyrosine, D-phenylalanine, and N-acetyl-phenylalanine.

495. The composition of any of embodiments 485-494, wherein the T-amino acid-entity is present, e.g., the T-amino acid-entity is present in amount of at least 0.5 wt. %, at least 1 wt. %, at least 1.5 wt. %, at least 2 wt. %, at least 2.5%, or at least 3 wt. % of the composition.

496. The composition of embodiment 495, wherein the T-amino acid-entity is selected from the group consisting of a precursor, a metabolite, and a derivative.

497. The composition of embodiment 495 or 496, wherein the T-amino acid-entity is selected from the group consisting of L-threonine, homoserine, O-phosphohomoserine, oxobutyrate, D-threonine, and N-acetyl-threonine.

498. The composition of any of embodiments 485-497, wherein the H-amino acid entity, the K-amino acid entity, the F-amino acid entity, and the T-amino acid entity are present in the composition.

499. The composition of any of embodiments 1-483, wherein the composition further comprises EAA-entities.

500. The composition of embodiment 499, wherein the EAA-entities are chosen from one, two, three, or four of a H-amino acid-entity, a K-amino acid-entity, a F-amino acid-entity, and a T-amino acid-entity and a protein source of EAAs.

501. The composition of embodiment 499 or 500, wherein the EAA-entities comprise a H-amino acid-entity, a K-amino acid-entity, a F-amino acid-entity, and a T-amino acid-entity.

502. The composition of any of embodiments 1-483, wherein the composition further comprises a protein source of EAAs instead of EAA-entities.

503. The composition of any of embodiments 1-483, wherein the composition does not comprises a protein source of EAAs.

504. A composition comprising:

a) a L-amino acid entity chosen from L-leucine or a salt thereof, or β-hydroxy-β-methybutyrate (HMB) or a salt thereof or a combination of L-leucine or a salt thereof and HMB and/or a salt thereof;

b) an R-amino acid entity chosen from L-arginine or a salt thereof, ornithine or a salt thereof, or creatine or a salt thereof or a combination of two or three of L-arginine or a salt thereof, ornithine or a salt thereof, or creatine or a salt thereof; and

c) L-glutamine or a salt thereof;

d) N-acetylcysteine (NAC) or a salt thereof; and

e) an EAA chosen from L-histidine or a salt thereof, L-lysine or a salt thereof, L-phenylalanine or a salt thereof, or L-threonine or a salt thereof, or a combination of two, three, or four of the EAAs.

505. The composition of any of embodiments 1-73 or 504, wherein the L-leucine is provided as part of a dipeptide comprising L-leucine, or a salt thereof, or a tripeptide comprising L-leucine, or a salt thereof.

506. The composition of any of embodiments 1-73, 504, or 505, wherein the L-arginine is provided as part of a dipeptide comprising L-arginine, or a salt thereof, or a tripeptide comprising L-arginine, or a salt thereof.

507. The composition of any of embodiments 1-13, 25, 29, 40, 54, 58, 62, 64, 68, 86, 102, 116, 120, 124, 126, 130, 147, 163, 177, 181, 185, 187, 191, 208, 224, 238, 242, or 504-506, wherein the L-glutamine is provided as part of a dipeptide comprising L-glutamine, or a salt thereof, or a tripeptide comprising L-glutamine, or a salt thereof.

508. The composition of any of embodiments 504-507, wherein the NAC is provided as part of a dipeptide comprising NAC, or a salt thereof, or a tripeptide comprising NAC, or a salt thereof.

509. The composition of any of the preceding embodiments, wherein the L-histidine is provided as part of a dipeptide comprising L-histidine, or a salt thereof, or a tripeptide comprising L-histidine, or a salt thereof.

510. The composition of any of any of the preceding embodiments, wherein the L-lysine is provided as part of a dipeptide comprising L-lysine, or a salt thereof, or a tripeptide comprising L-lysine, or a salt thereof.

511. The composition of any of any of the preceding embodiments, wherein the L-phenylalanine is provided as part of a dipeptide comprising L-phenylalanine, or a salt thereof, or a tripeptide comprising L-phenylalanine, or a salt thereof.

512. The composition of any of any of the preceding embodiments, wherein the L-threonine is provided as part of a dipeptide comprising L-threonine, or a salt thereof, or a tripeptide comprising L-threonine, or a salt thereof.

513. The composition of any of the preceding embodiments, wherein at least three or four of the amino acids of (a)-(d) is not provided as a peptide of more than 20 amino acid residues in length.

514. The composition of any of the preceding embodiments, wherein one, two, three, or four of methionine (M), tryptophan (W), valine (V), or cysteine (C) is absent, or if present, is present at less than 10 weight (wt.) % of the composition.

515. The composition of any of the preceding embodiments, wherein the total wt. % of (a)-(e) is greater than the total wt. % of any other amino acid entity in the composition.

516. The composition of any of the preceding embodiments, wherein one, two, three, four, or five of (a)-(e) is provided as a dipeptide or tripeptide, e.g., in an amount of at least 10 wt. % of the composition.

517. The composition of embodiment 516, wherein the dipeptide is a homodipeptide or heterodipeptide of any of (a)-(e), e.g., one, two, three, or four of (a)-(e) is a homodipeptide or heterodipeptide.

518. The composition of embodiment 516, wherein the tripeptide is a homotripeptide or heterotripeptide of any of (a)-(e), e.g., one, two, three, or four of (a)-(e) is a homotripeptide or heterotripeptide.

519. The composition of any of the preceding embodiments, wherein (a) is a L-amino acid entity dipeptide or a salt thereof (e.g., a L-leucine dipeptide or a salt thereof).

520. The composition of embodiment 519, wherein (a) is a homodipeptide or a heterodipeptide, e.g., Ala-Leu.

521. The composition of any of the preceding embodiments, wherein (b) is a L-arginine dipeptide or a salt thereof.

522. The composition of embodiment 521, wherein (b) is a homodipeptide or a heterodipeptide, e.g., Ala-Arg.

523. The composition of any of the preceding embodiments, wherein (c) is a L-glutamine dipeptide or a salt thereof.

524. The composition of embodiment 523, wherein (c) is a homodipeptide, e.g., Gln-Gln, or wherein (c) is a heterodipeptide, e.g., Ala-Gln.

525. The composition of any of the preceding embodiments, wherein:

f) a wt. % of the R-amino acid entity in the composition is greater than the wt. % of the L-glutamine or a salt thereof;

g) the wt. % of the L-glutamine or a salt thereof in the composition is greater than the wt. % of the L-amino acid entity;

h) the wt. % of the R-amino acid entity in the composition is greater than the wt. % of the L-amino acid entity;

i) the wt. % of the R-amino acid entity in the composition is greater than the wt. % of the EAA, or the combination of two, three, or four of the EAAs;

j) the wt. % of the L-glutamine or a salt thereof in the composition is greater than the wt. % of the EAA or the combination of two, three, or four of the EAAs;

k) the wt. % of the L-amino acid entity in the composition is greater than the wt. % of the EAA or the combination of two, three, or four of the EAAs; or

l) a combination of two, three, four, five, or six of (f)-(k).

526. The composition of any of the preceding embodiments, wherein the wt. % of the R-amino acid entity in the composition is at least 2% greater than the wt. % of the L-glutamine or a salt thereof, e.g., the wt. % of the L-glutamine or a salt thereof is at least 3%, 4%, 5%, 6%, 7%, 8%, 9%, or 10% greater than the wt. % of the R-amino acid entity

527. The composition of any of the preceding embodiments, wherein the wt. % of the L-glutamine or a salt thereof in the composition is at least 10% greater than the wt. % of the L-amino acid entity, e.g., the wt. % of the L-glutamine or a salt thereof in the composition is at least 12%, 15%, 20%, 22%, or 25% greater than the wt. % of the L-amino acid entity.

528. The composition of any of the preceding embodiments, wherein the wt. % of the R-amino acid entity in the composition is at least 10% greater than the wt. % of the L-amino acid entity, e.g., the wt. % of the R-amino acid entity in the composition is at least 15%, 20%, 25%, or 30% greater than the wt. % of the L-amino acid entity.

529. The composition of any of the preceding embodiments, wherein the wt. % of the R-amino acid entity in the composition is at least 25% greater than the wt. % of the EAA or the combination of two, three, or four of the EAAs, e.g., the wt. % of the R-amino acid entity in the composition is at least 20%, 30%, 40%, or 50% greater than the wt. % of the EAA or the combination of two, three, or four of the EAAs.

530. The composition of any of the preceding embodiments, wherein the wt. % of the L-glutamine or a salt thereof in the composition is at least 25% greater than the wt. % of the EAA or the combination of two, three, or four of the EAAs, e.g., the wt. % of the L-glutamine or a salt thereof in the composition is at least 20%, 30%, 40%, or 50% greater than the wt. % of the EAA or the combination of two, three, or four of the EAAs.

531. The composition of any of the preceding embodiments, wherein the wt. % of the L-amino acid entity in the composition is at least 10% greater than the wt. % of the EAA or the combination of two, three, or four of the EAAs, e.g., the wt. % of the L-glutamine or a salt thereof in the composition is at least 12%, 15%, 20%, 22%, or 25% greater than the wt. % of the EAA or the combination of two, three, or four of the EAAs.

532. The composition of any of the preceding embodiments, wherein:

m) the ratio of the L-amino acid entity to the R-amino acid entity is at least 1:4, or at least 2:5, and not more than 3:4, e.g., the ratio of L-amino acid entity to R-amino acid entity is about 2:3;

n) the ratio of the L-amino acid entity to the L-glutamine or a salt thereof is at least 1:4, or least 1:3, and not more than 3:4, e.g., the ratio of the L-amino acid entity to the L-glutamine or a salt thereof is about 2:3;

o) the ratio of the L-glutamine or a salt thereof to the R amino acid entity is at least 1:2, or least 3:4, and not more than 11:12, e.g., the ratio of the L-glutamine or a salt thereof to the R-amino acid entity is about 8:9;

p) the ratio of the EAA, or the combination of two, three, or four of the EAAs, to the L-amino acid entity is at least 1:4, or at least 2:5, and not more than 3:4, e.g., the ratio of the EAA, or the combination of two, three, or four of the EAAs, to the L-amino acid entity is about 2:3;

q) the ratio of the EAA, or the combination of two, three, or four of the EAAs, to the L-glutamine or a salt thereof is at least 1:4, or at least 2:5, and not more than 3:4, e.g., the ratio of the EAA, or the combination of two, three, or four of the EAAs, to the L-glutamine or a salt thereof is about 1:2;

r) the ratio of the EAA to the R-amino acid entity is at least 1:5, or at least 1:3, and not more than 2:3, e.g., the ratio of the EAA, or the combination of two, three, or four of the EAAs, to the R-amino acid entity is about 4:9; or

s) a combination of two, three, four, five, or six of (m)-(r).

533. The composition of any of the preceding embodiments, further comprising one or both of an isoleucine (I)-amino acid-entity and a valine (V)-amino acid-entity, e.g., both the I-amino acid-entity and the V-amino acid-entity are present.

534. The composition of embodiment 533, wherein:

t) the wt. % of the L-amino acid-entity in the composition is greater than or equal to the wt. % of the I-amino acid-entity and the V-amino acid-entity in combination;

u) the wt. % of the L-amino acid-entity, the I-amino acid-entity, and the V-amino acid-entity in combination in the composition is greater than or equal to the wt. % of the L-glutamine or a salt thereof;

v) the wt. % of the L-amino acid-entity, the I-amino acid-entity, and the V-amino acid-entity in combination in the composition is less than the wt. % of the R-amino acid entity;

w) the wt. % of the R-amino acid entity and the L-glutamine or a salt thereof in the composition is greater than the wt. % of the L-amino acid-entity, the I-amino acid-entity, and the V-amino acid-entity in combination;

x) the wt. % of the L-amino acid-entity, the I-amino acid-entity, and the V-amino acid-entity in combination is greater than the EAA, or the combination of two, three, or four of the EAAs, in the composition;

y) the wt. % of the I-amino acid-entity in combination with the L-amino acid entity or the V-amino acid-entity is greater than the EAA, or the combination of two, three, or four of the EAAs, in the composition;

aa) the wt. % of the V-amino acid entity is greater than the EAA, or the combination of two, three, or four of the EAAs, in the composition; or

y) a combination of two, three, four, five, six, seven, or eight of (t)-(x).

535. The composition of embodiment 533 or 534, wherein:

z) the wt. % of the R-amino acid entity, the L-glutamine or a salt thereof, and the NAC or a salt thereof is at least 30% of the composition, or at least 40% of the composition, but not more than 70% of the composition;

aa) the wt. % of the NAC or a salt thereof is at least 1%, or at least 2%, but not more than 10% of the composition;

bb) the wt. % of the L-amino acid-entity, the I-amino acid-entity, and the V-amino acid-entity in combination is at least 20%, or at least 25%, but not more than 60% of the composition;

cc) the wt. % of the R-amino acid entity, the L-glutamine or a salt thereof, and the NAC or a salt thereof is at least 40%, or at least 50%, but not more than 80% of the composition;

dd) the wt. % of the EAA, or the combination of two, three, or four of the EAAs, in the composition is at least 5%, or at least 10%, but not more than 25%, e.g., the wt. % of the EAA, or the combination of two, three, or four of the EAAs, is about 12% or about 14%; or

ee) a combination of two, three, four, or five of (z)-(dd).

536. The composition of any of embodiments 533-535, wherein:

ff) the ratio of the L-amino acid entity to the I-amino acid entity is at least 3:2, or at least 7:4, and not more than 5:2 or not more than 3:1, e.g., the ratio of the L-amino acid entity to the I-amino acid entity is about 2:1;

gg) the ratio of L-amino acid entity to V-amino acid entity is at least 3:2, or at least 7:4, and not more than 5:2 or not more than 3:1, e.g., the ratio of L to V is about 2:1;

hh) the ratio of the L-amino acid entity to the R-amino acid entity is greater than 1:3, greater than 1:2, and less than 3:4, e.g., the ratio of the L-amino acid entity to the R-amino acid entity is about 2:3;

ii) the ratio of the L-amino acid entity to the L-glutamine or a salt thereof is greater than 1:4, greater than 3:8, and less than 5:6, or less than 6:7, e.g., the ratio of the L-amino acid entity to the L-glutamine or a salt thereof is about 3:4;

jj) the ratio of the EAA, or the combination of two, three, or four of the EAAs, to the L-amino acid is greater than 1:4, greater than 3:8, and less than 3:4, or less than 5:6, e.g., the ratio of the EAA, or the combination of two, three, or four of the EAAs, to the L-amino acid entity is about 2:3; or

kk) a combination of two, three, four, or five of (ff)-(jj).

537. The composition of any of embodiments 533-536, wherein:

ll) the ratio of the I-amino acid entity to the V-amino acid entity is at least 0.5:1, or at least 0.75:1, and not more than 1.5 to 1 or not more than 2:1, e.g., the ratio of the L-amino acid entity to the I-amino acid entity is about 1:1;

mm) the ratio of the I-amino acid entity to the R-amino acid entity is at least 1:6, or at least 0.75:3, and not more than 2:3, or not more than 1.5:3, e.g., the ratio of the L-amino acid entity to the I-amino acid entity is about 1:3;

nn) the ratio of the I-amino acid entity to the L-glutamine or a salt thereof is at least 1:8, or at least 1:4, and not more than 3:4, or not more than 1:2, e.g., the ratio of the L-amino acid entity to the L-glutamine or a salt thereof is about 3:8;

oo) the ratio of the I-amino acid to the EAA, or the combination of two, three, or four of the EAAs, to is greater than 1:3, greater than 1:2, and less than 5:6, or less than 6:7, e.g., the ratio of the I-amino acid entity to the EAA, or the combination of two, three, or four of the EAAs, is about 3:4; or

pp) a combination of two, three, or four of (ll)-(oo).

538. The composition of any of embodiments 533-537, wherein:

qq) the ratio of the L-amino acid entity to the V-amino acid entity is at least 3:2, or at least 7:4, and not more than 3:1 or not more than 4:1, e.g., is the ratio of the L-amino acid entity to the V-amino acid entity is about 2:1;

rr) the ratio of the L-amino acid entity to the R-amino acid entity is greater than 1:3, greater than 3:6, and less than 3:4, e.g., the ratio of the L-amino acid entity to the R-amino acid entity is about 2:3;

ss) the ratio of the L-amino acid entity to the L-glutamine or a salt thereof is greater than 1:4, greater than 1:2 and less than 5:6, or less than 6:7, e.g., the ratio of the L-amino acid entity to the L-glutamine or a salt thereof is about 3:4;

tt) the ratio of the I-amino acid to the EAA, or the combination of two, three, or four of the EAAs, to is greater than 1:3, greater than 1:2, and less than 5:6, or less than 6:7, e.g., the ratio of the I-amino acid entity to the EAA, or the combination of two, three, or four of the EAAs, is about 3:4; or

uu) a combination of two, three, or four of (qq)-(tt).

539. The composition of any of embodiments 533-538, wherein:

vv) the ratio of the V-amino acid entity to the L-glutamine or a salt thereof is at least 1:8, or at least 1:4, and not more than 3:4, or not more than 1:2, e.g., the ratio of the L-amino acid entity to the L-glutamine or a salt thereof is about 3:8;

ww) the ratio of the V-amino acid entity to the R-amino acid entity is at least 1:9, or at least 2:9, and not more than 2:3, or not more than 1:2, e.g., the ratio of the V-amino acid entity to the R-amino acid entity is 1:3;

xx) the ratio of the L-amino acid-entity, the I-amino acid-entity, and the V-amino acid-entity in combination to the R-amino acid entity, L-glutamine or a salt thereof, and NAC or a salt thereof is at least 1:4, or at least 1:3, and not more than 7:9, or not more than 8:9, e.g., the ratio is about 6:9;

yy) the ratio of the EAA, or the combination of two, three, or four of the EAAs, to the L-amino acid-entity, the I-amino acid-entity, and the V-amino acid-entity in combination to is at least 1:5, or at least 1:4, and not more than 2:3, or not more than 3:4, e.g., the ratio is about 1:3; or

zz) a combination of two, three, or four of (vv)-(yy).

540. The composition of any of the preceding embodiments, wherein:

aaa) a wt. % of the L-amino acid entity in the composition is greater than the wt. % of the NAC or a salt thereof;

bbb) a wt. % of the R-amino acid entity in the composition is greater than the wt. % of the NAC or a salt thereof;

ccc) a wt. % of the L-glutamine or a salt thereof in the composition is greater than the wt. % of the NAC or a salt thereof; or

ddd) a combination of two or three of (aaa)-(ccc).

541. The composition of any of the preceding embodiments, wherein at least one of (a)-(d) is a free amino acid, e.g., two, three, or four of (a)-(d) are a free amino acid.

542. The composition of claim 541, wherein at least 50 wt. % of the total wt. of the composition is one or more amino acid entities in free form.

543. The composition of any of the preceding embodiments, wherein at least one of (a)-(d) is in a salt form, e.g., one, two, three, or four of (a)-(d) is in a salt form.

544. The composition of claim 541, wherein at least 10 wt. % of the total wt. of the composition is one or more amino acid entities in a salt form.

545. The composition of any of the preceding embodiments, wherein the composition is are capable of one, two, three, four or all of:

a) activating mTORC1;

b) activating protein synthesis and/or inhibiting protein catabolism;

c) improving, e.g., increasing, insulin sensitivity or glucose tolerance;

d) reducing inflammation; or

e) improving or increasing myogenesis.

546. The composition of any of the preceding embodiments, wherein the wt. ratio of the L-amino acid entity, the R-amino acid entity, the L-glutamine or a salt thereof, and the NAC or a salt thereof is about 1-3:2-4:2-4:0.1-1.5, e.g., the wt. ratio of the L-amino acid entity, the I-amino acid entity, the V-amino acid entity, the R-amino acid entity, the L-glutamine or a salt thereof, the NAC or a salt thereof, the L-histidine or a salt thereof, the L-lysine or a salt thereof, the L-phenylalanine or a salt thereof, and the L-threonine or a salt thereof entity is about 1-3:0.5-1.5:0.5-1.5:2-4:2-4:0.1-1.5:0.1-0.5:0.2-1.0:0.1-0.5:0.2-0.7.

547. The composition of any of the preceding embodiments, wherein the composition comprises about 0.5 g to about 15 g of the L-amino acid entity, about 0.25 g to about 10 g of the I-amino acid entity, about 0.25 g to about 10 g of the V-amino acid entity, about 0.5 to about 25 g of the R-amino acid entity, about 0.5 g to about 20 g of the L-glutamine or a salt thereof, about 0.1 to about 5 g the NAC or a salt thereof, about 0.05 g to about 3 g of the L-histidine or a salt thereof, about 0.05 to about 6 g of the L-lysine or a salt thereof, about 0.04 to about 2 g of the L-phenylalanine or a salt thereof, and about 0.08 to about 4 g of the L-threonine or a salt thereof entity; e.g., about 1 g of the L-amino acid entity, about 0.5 g of the I-amino acid entity, about 0.5 g of the V-amino acid entity, about 1.5 g or about 1.81 of the R-amino acid entity, about 1.33 g of the L-glutamine or a salt thereof, about 0.15 g or about 0.3 g of the NAC or a salt thereof, about 0.08 g of the L-histidine or a salt thereof, about 0.35 g of the L-lysine or a salt thereof, about 0.08 g of the L-phenylalanine or a salt thereof, and about 0.17 g of the L-threonine or a salt thereof.

548. A method for improving muscle function, wherein the method comprises administering to a composition of any of the preceding embodiments

549. The method of embodiment 548, wherein the L-leucine is provided as part of a dipeptide comprising L-leucine, or a salt thereof, or a tripeptide comprising L-leucine, or a salt thereof.

550. The method of embodiment 548 or 549, wherein the L-arginine is provided as part of a dipeptide comprising L-arginine, or a salt thereof, or a tripeptide comprising L-arginine, or a salt thereof.

551. The method of any of embodiments 548-550, wherein the L-glutamine is provided as part of a dipeptide comprising L-glutamine, or a salt thereof, or a tripeptide comprising L-glutamine, or a salt thereof.

552. The method of any of embodiments 548-551, wherein the NAC is provided as part of a dipeptide comprising NAC, or a salt thereof, or a tripeptide comprising NAC, or a salt thereof.

553. The method of any of embodiments 548-552, wherein the L-histidine is provided as part of a dipeptide comprising L-histidine, or a salt thereof, or a tripeptide comprising L-histidine, or a salt thereof.

554. The method of any of embodiments 548-553, wherein the L-lysine is provided as part of a dipeptide comprising L-lysine, or a salt thereof, or a tripeptide comprising L-lysine, or a salt thereof.

555. The method of any of embodiments 548-554, wherein the L-phenylalanine is provided as part of a dipeptide comprising L-phenylalanine, or a salt thereof, or a tripeptide comprising L-phenylalanine, or a salt thereof.

556. The method of any of embodiments 548-555, wherein the L-threonine is provided as part of a dipeptide comprising L-threonine, or a salt thereof, or a tripeptide comprising L-threonine, or a salt thereof.

557. A method for treating one or more symptoms selected from immobilization, malnutrition, fasting, aging, autophagy, reduced protein synthesis, anabolic resistance, junction integrity, insulin resistance, decreased mitochondrial biogenesis, anaplerosis, or an energy deficit, wherein the method comprises administering to a subject in need thereof an effective amount of a composition comprising:

a) a L-amino acid entity chosen from L-leucine or a salt thereof, or β-hydroxy-β-methybutyrate (HMB) or a salt thereof;

b) an R-amino acid entity chosen from L-arginine or a salt thereof, ornithine or a salt thereof, or creatine or a salt thereof; and

c) L-glutamine or a salt thereof;

d) N-acetylcysteine (NAC) or a salt thereof; and

e) an EAA chosen from L-histidine or a salt thereof, L-lysine or a salt thereof, L-phenylalanine or a salt thereof, or L-threonine or a salt thereof or a combination of two, three, or four of the EAAs.

558. The method of embodiment 557, wherein the L-leucine is provided as part of a dipeptide comprising L-leucine, or a salt thereof, or a tripeptide comprising L-leucine, or a salt thereof.

559. The method of embodiment 557 or 558, wherein the L-arginine is provided as part of a dipeptide comprising L-arginine, or a salt thereof, or a tripeptide comprising L-arginine, or a salt thereof.

560. The method of any of embodiments 557-559, wherein the L-glutamine is provided as part of a dipeptide comprising L-glutamine, or a salt thereof, or a tripeptide comprising L-glutamine, or a salt thereof.

561. The method of any of embodiments 557-560, wherein the NAC is provided as part of a dipeptide comprising NAC, or a salt thereof, or a tripeptide comprising NAC, or a salt thereof.

562. The method of any of embodiments 557-561, wherein the L-histidine is provided as part of a dipeptide comprising L-histidine, or a salt thereof, or a tripeptide comprising L-histidine, or a salt thereof.

563. The method of any of embodiments 557-562, wherein the L-lysine is provided as part of a dipeptide comprising L-lysine, or a salt thereof, or a tripeptide comprising L-lysine, or a salt thereof.

564. The method of any of embodiments 557-563, wherein the L-phenylalanine is provided as part of a dipeptide comprising L-phenylalanine, or a salt thereof, or a tripeptide comprising L-phenylalanine, or a salt thereof.

565. The method of any of embodiments 557-564, wherein the L-threonine is provided as part of a dipeptide comprising L-threonine, or a salt thereof, or a tripeptide comprising L-threonine, or a salt thereof.

566. A method of improving or increasing myogenesis, wherein the method comprises administering to a subject in need thereof an effective amount of a composition of any of the preceding embodiments.

567. The method of embodiment 566, wherein the L-leucine is provided as part of a dipeptide comprising L-leucine, or a salt thereof, or a tripeptide comprising L-leucine, or a salt thereof.

568. The method of embodiment 566 or 567, wherein the L-arginine is provided as part of a dipeptide comprising L-arginine, or a salt thereof, or a tripeptide comprising L-arginine, or a salt thereof.

569. The method of any of embodiments 566-568, wherein the L-glutamine is provided as part of a dipeptide comprising L-glutamine, or a salt thereof, or a tripeptide comprising L-glutamine, or a salt thereof.

570. The method of any of embodiments 566-569, wherein the NAC is provided as part of a dipeptide comprising NAC, or a salt thereof, or a tripeptide comprising NAC, or a salt thereof.

571. The method of any of embodiments 566-570, wherein the L-histidine is provided as part of a dipeptide comprising L-histidine, or a salt thereof, or a tripeptide comprising L-histidine, or a salt thereof.

572. The method of any of embodiments 566-571, wherein the L-lysine is provided as part of a dipeptide comprising L-lysine, or a salt thereof, or a tripeptide comprising L-lysine, or a salt thereof.

573. The method of any of embodiments 566-572, wherein the L-phenylalanine is provided as part of a dipeptide comprising L-phenylalanine, or a salt thereof, or a tripeptide comprising L-phenylalanine, or a salt thereof.

574. The method of any of embodiments 566-573, wherein the L-threonine is provided as part of a dipeptide comprising L-threonine, or a salt thereof, or a tripeptide comprising L-threonine, or a salt thereof.

575. The method of any of embodiments 566-574, wherein the subject has a disease or disorder selected from the group consisting of a rare muscle disease, muscle atrophy, sarcopenia, muscle deterioration, muscle decay, cachexia, drug-induced myopathy, muscular dystrophy, myopenia, muscle weakness, perceived muscle weakness, ICU-acquired myopathy, burns-related myopathy, a neuromuscular disorder, ventilator-induced diaphragmatic dystrophy, ventilator-induced diaphragmatic dysfunction, hyponatremia, hypokalemia, a calcium deficiency, hypercalcemia, amyotrophic lateral sclerosis, and a bone weakness disease.

576. The method of any of embodiments 566-575, wherein the subject has or is identified as having decreased muscle function due to aging, injury, muscle atrophy, infection, disease, stroke, or a fracture or other trauma.

577. The method of any of embodiments 566-576, wherein the subject has had a rotator cuff surgery, knee surgery, hip surgery, joint replacement, injury repair surgery, or has worn a cast prior to administration of the composition.

578. A composition comprising free amino acids, wherein the amino acids comprise arginine, glutamine, N-acetylcysteine; a branched-chain amino acid chosen from one, two, or all of leucine, isoleucine, and valine; and an essential amino acid chosen from one, two, three, or all of histidine, lysine, phenylalanine and threonine.

579. The composition of embodiment 578, wherein the branched-chain amino acid is leucine, isoleucine, and valine.

580. The composition of embodiment 578, wherein the essential amino acid is histidine, lysine, phenylalanine, and threonine.

581. The composition of any of the preceding embodiments, wherein the composition comprises a ratio of branched-chain amino acids to total amino acids of about 4:7 to about 1:2.

582. The composition of any of embodiments 578-581, wherein the weight (wt.) ratio of leucine, isoleucine, valine, arginine, glutamine, N-acetylcysteine, histidine, lysine, phenylalanine, and threonine is about 2.0:1.0:1.0:3.0:2.66:0.3:0.16:0.7:0.16:0.34.

583. The composition of any of the preceding embodiments, wherein the total wt. of amino acids present is between about 4 g and about 80 g.

584. The composition of embodiment 583, wherein the total wt. of amino acids present is about 6 g, about 18 g, about 24 g, or about 72 g.

585. The composition of any of embodiments 578-584, wherein the composition comprises at least 1 g of leucine, at least 0.5 g of isoleucine, at least 0.5 g of valine, at least 1.5 g of arginine, at least 1.33 g of glutamine, at least 0.15 g of N-acetylcysteine, at least 0.08 g of histidine, at least 0.35 g of lysine, at least 0.08 g of phenylalanine, and at least 0.17 g of threonine.

586. The composition of any of embodiments 578-584, wherein the composition comprises at least 3 g of leucine, at least 1.5 g of isoleucine, at least 1.5 g of valine, at least 4.5 g of arginine, at least 3.99 g of glutamine, at least 0.45 g of N-acetylcysteine, at least 0.24 g of histidine, at least 1.05 g of lysine, at least 0.24 g of phenylalanine, and at least 0.51 g of threonine.

587. The composition of embodiments 578-584, wherein the amino acids comprise about 10 wt % to about 20 wt % leucine, about 5 wt % to about 15 wt % isoleucine, about 5 wt % to about 15 wt % valine, about 20 wt % to about 40 wt % arginine, about 15 wt % to about 35 wt % glutamine, about 1 wt % to about 10 wt % N-acetylcysteine, about 0.5 wt % to about 5 wt % histidine, about 3 wt % to about 8 wt % lysine, about 0.5 wt % to about 5 wt % phenylalanine, and about 1 wt % to about 8 wt % threonine.

588. The composition of any of the preceding embodiments, wherein the composition further comprises one or more pharmaceutically acceptable excipients.

589. The composition of any of embodiments 578-588, wherein the amino acids consist of leucine, isoleucine, valine, arginine, glutamine, N-acetylcysteine, histidine, lysine, phenylalanine, and threonine.

590. A method for treating one or more symptoms selected from the group consisting of immobilization, malnutrition, fasting, aging, autophagy, reduced protein synthesis, anabolic resistance, neuromuscular junction integrity, insulin resistance, decreased mitochondrial biogenesis, and anaplerosis, wherein the method comprises administering to a subject in need thereof an effective amount of the composition of any of the preceding embodiments.

591. The method of embodiment 590, wherein the subject has a rare muscle disease.

592. The method of embodiment 590 or 591, wherein the subject has muscle deterioration, muscle decay, muscle atrophy, cachexia, sarcopenia, drug-induced myopathy, muscular dystrophy, or myopenia.

593. A method for enhancing muscle function comprising administering to a subject in need thereof an effective amount of a composition of the preceding embodiments

594. The method of embodiment 593, wherein the subject has or is identified as having decreased muscle function due to aging, injury, atrophy, infection, or disease.

595. The method of embodiment 593 or 594, wherein the subject has or is identified as having muscle deterioration, muscle decay, muscle atrophy, cachexia, sarcopenia, drug-induced myopathy, muscular dystrophy, or myopenia.

596. The method of any of embodiments 590-595, wherein administration of the composition results in an improvement in one or more metabolic symptoms in the subject.

597. The method of embodiment 596, wherein the improvement in one or more metabolic symptoms is selected from the following: mTORC1 activation; improved insulin sensitivity; activation of muscle protein synthesis; scavenging of reactive oxygen species (ROS); decreased inflammation; inhibition of catabolism; ammonia detoxification; and decreased fibrosis progression.

598. The method of any of embodiments 590-597, wherein administration of the composition reduces muscle atrophy in the subject.

599. The method of any of embodiments 590-598, wherein administration of the composition results in anabolism and catabolism of muscle tissue.

600. The method of any of embodiments 590-599, wherein the subject is a human.

601. A dietary composition comprising the composition of any of embodiments 578-589, e.g., wherein the dietary composition is chosen from a medical food, a functional food, or a supplement.

602. The composition of any of embodiments 578-589 for use as a dietary composition, e.g., wherein the dietary composition is chosen from a medical food, a functional food, or a supplement.

603. The dietary composition for use of embodiment 602, wherein the composition is for use in treating a subject having or identified as having decreased muscle function due to aging, injury, atrophy, infection, or disease.

604. The dietary composition for use of embodiment 603, wherein the subject has or is identified as having muscle deterioration, muscle decay, muscle atrophy, cachexia, sarcopenia, drug-induced myopathy, or muscular dystrophy.

605. A pharmaceutical composition comprising the composition of any of embodiments 1-589.

606. The composition of any of embodiments 1-13 or 504-605, wherein the L-amino acid entity is chosen from the group consisting of L-leucine, β-hydroxy-β-methybutyrate (HMB), oxo-leucine, isovaleryl-CoA, D-leucine, and n-acetyl-leucine, or a combination thereof.

607. The composition of any of embodiments 1-13 or 504-606, wherein the R-amino acid entity is chosen from the group consisting of L-arginine, ornithine, argininosuccinate, citrulline, aspartate, glutamate, agmatine, creatine, D-arginine, and N-acetyl-arginine, or a combination thereof.

608. The composition of any of embodiments 1-13 or 504-607, wherein the Q-amino acid entity is chosen from the group consisting of L-glutamine, glutamate, carbamoyl-P, glutamate, D-glutamine, and n-acetylglutamine, or a combination thereof.

609. The composition of any of embodiments 1-13 or 504-608, wherein the NAC-amino acid entity is chosen from the group consisting of NAC, serine, acetylserine, cystathionine, glutathione, homocysteine, methionine, D-cysteine, L-cysteine, cysteamine, and cystine, or a combination thereof.

610. The composition of any of embodiments 1-13 or 504-610, wherein the H-amino acid entity is chosen from the group consisting of L-histidine, histidinol, histidinal, ribose-5-phosphate, carnosine, histamine, urocanate, D-histidine, and N-acetyl-histidine, or a combination thereof.

611. The composition of any of embodiments 1-13 or 504-610, wherein the K-amino acid entity is chosen from the group consisting of L-lysine, diaminopimelate, aspartate, trimethyllysine, carnitine, saccharopine, D-lysine, and N-acetyl-lysine, or a combination thereof.

612. The composition of any of embodiments 1-13 or 504-611, wherein the F-amino acid entity is chosen from the group consisting of L-phenylalanine, phenylpyruvate, tyrosine, D-phenylalanine, and N-acetyl-phenylalanine, or a combination thereof.

613. The composition of any of embodiments 1-13 or 504-612, wherein the T-amino acid entity is chosen from the group consisting of L-threonine, homoserine, O-phosphohomoserine, oxobutyrate, D-threonine, and N-acetyl-threonine, or a combination thereof.

614. A dietary composition comprising the composition of any of the preceding embodiments, wherein the dietary compositions is chosen from a medical food, a functional food, or a supplement.

615. A method of providing amino acid entities to a subject comprising administering to the subject an effective amount of the composition of any of the preceding embodiments.

617. A method of manufacturing or making a composition comprising forming a composition comprising the following:

a) a L-amino acid entity,

b) an R-amino acid entity,

c) a Q-amino acid entity;

d) a NAC entity, e.g., NAC; and

e) an EAA-entity chosen from a H-amino acid-entity, a K-amino acid-entity, a F-amino acid-entity, and a T-amino acid-entity or a combination of two, three, or four; provided that:

f) at least one amino acid entity is not a peptide of more than 20 amino acid residues in length wherein:

(i) an amino acid entity of (a) is selected from Table 2; and

(ii) one or both of the R-amino acid entity and the Q-amino acid entity are present at a higher amount (wt. %) than the L-amino acid entity.

618. The composition or method of any of the preceding embodiments, wherein the composition is capable of activating mTORC1 by at least 20%, 25%, 30%, 35%, 40%, 45%, 50%, 55%, 60%, 65%, 70%, 75%, 80%, 85%, 90%, 95%, or 99%, as detected using as an assay to measure mTORC1 substrate phosphorylation, e.g., P-rpS6 phosphorylation, e.g., an ELISA and/or cellular kinase assay, e.g., as described in Example 1, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-arginine, L-glutamine, and NAC; L-glutamine; or NAC).

619. The composition or method of any of the preceding embodiments, wherein the composition is capable of phosphorylating an mTORC1 substrate e.g., P-rpS6 phosphorylation by at least 20%, 25%, 30%, 35%, 40%, 45%, 50%, 55%, 60%, 65%, 70%, 75%, 80%, 85%, 90%, 95%, or 99%, as detected using as assay to measure mTORC1 substrate phosphorylation, e.g., P-rpS6 phosphorylation, e.g., an ELISA and/or cellular kinase assay, e.g., as described in Example 1, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-arginine, L-glutamine, and NAC; L-glutamine; or NAC).

620. The composition or method of any of the preceding embodiments, wherein the composition is capable of increasing myogenesis by at least 20%, 25%, 30%, 35%, 40%, 45%, 50%, 55%, 60%, 65%, 70%, 75%, 80%, 85%, 90%, 95%, or 99%, as detecting by counting myoblasts cells, e.g., C2C12 cells, e.g., by a nuclear stain, e.g., a Hoechst stain, e.g., as described in Example 2, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-leucine, L-isoleucine, L-valine, L-arginine, and NAC; L-glutamine, and NAC; L-glutamine; NAC; or an amino acid composition comprising L-leucine, L-arginine, L-glutamine, NAC, L-histidine, L-lysine, L-phenylanine, and L-threonine).

621. The composition or method of any of the preceding embodiments, wherein the composition is capable of increasing myoblast cell count by at least 20%, 25%, 30%, 35%, 40%, 45%, 50%, 55%, 60%, 65%, 70%, 75%, 80%, 85%, 90%, 95%, or 99%, as detecting by counting myoblasts cells, e.g., C2C12 cells, e.g., by a nuclear stain, e.g., a Hoechst stain, e.g., as described in Example 2, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-leucine, L-isoleucine, L-valine, L-arginine, and NAC; L-glutamine, and NAC; L-glutamine; NAC; or an amino acid composition comprising L-leucine, L-arginine, L-glutamine, NAC, L-histidine, L-lysine, L-phenylanine, and L-threonine).

622. The composition or method of any of the preceding embodiments, wherein the composition is capable of increasing myotube growth by at least 20%, 25%, 30%, 35%, 40%, 45%, 50%, 55%, 60%, 65%, 70%, 75%, 80%, 85%, 90%, 95%, or 99%, by detecting an increase of MyoD and/or Myogenin in, e.g., C2C12 cells, e.g., as detected using as immunohistochemistry, e.g., as described in Example 3, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-leucine, L-isoleucine, L-valine, L-arginine, and NAC; L-glutamine, and NAC; L-glutamine; NAC; or an amino acid composition comprising L-leucine, L-arginine, L-glutamine, NAC, L-histidine, L-lysine, L-phenylanine, and L-threonine).

623. The composition or method of any of the preceding embodiments, wherein the composition is capable of increasing MyoD and/or Myogenin by at least 20%, 25%, 30%, 35%, 40%, 45%, 50%, 55%, 60%, 65%, 70%, 75%, 80%, 85%, 90%, 95%, or 99%, as detecting by detecting an increase of MyoD and/or Myogenin in, e.g., C2C12 cells, e.g., as detected using as immunohistochemistry, e.g., as described in Example 3, e.g., relative to a reference composition (e.g., an amino acid composition comprising L-leucine, L-isoleucine, L-valine; an amino acid composition comprising L-leucine, L-isoleucine, L-valine, L-arginine, and L-glutamine; an amino acid composition comprising L-leucine, L-isoleucine, L-valine, L-arginine, and NAC; L-glutamine, and NAC; L-glutamine; NAC; or an amino acid composition comprising L-leucine, L-arginine, L-glutamine, NAC, L-histidine, L-lysine, L-phenylanine, and L-threonine).

624. The composition of any of the preceding embodiments for use as a medicament.

625. The composition of any of the preceding embodiments for use in a method as disclosed herein.

626. The use of a composition of any of the preceding embodiments in the manufacture of a medicament.

627. The use of a composition of any of the preceding embodiments in the manufacture of a medicament for treating any of the disorders or conditions disclosed herein.

Although many of the above embodiments are shown in dependent form, it is contemplated that any of the embodiments or combinations thereof may be in independent form.

### EXAMPLE

The Example below is set forth to aid in the understanding of the inventions, but is not intended to, and should not be construed to, limit its scope in any way.

### Example 1

### Activation of mTORC1 in Muscle Cells with an Amino Acid Composition

Metabolism is controlled by the mTORC1 signaling complex, an essential protein kinase that regulates cellular processes such as protein synthesis and autophagy. A number of distinct signals control mTORC1 activity, including amino acids and growth factors like insulin, and proper regulation is necessary for the maintenance of muscle mass. Dysregulation of mTORC1 activity is associated with muscle wasting (atrophy) in many diseases, and conversely, addition of muscle mass (hypertrophy) requires protein synthesis-inducing mTORC1 signaling. The ability of different amino acids to induce mTORC1 signaling in myotubes was assessed using an alpha-elisa screen for phosphorylated ribosomal protein S6 (P-rpS6), an important substrate downstream of mTORC1 involved in promoting protein synthesis.

In this example, murine muscle cells were incubated with a composition including amino acids and assessed for mTORC1 activation. C2C12, mouse muscle cells, were obtained from ATCC (CRL-1772, Manassas, Va.). The cells were seeded on day 0 at 1.0E4 cells per well in 96-well TC-treated microplates (Corning, Corning, N.Y.) in Dulbecco's Modified Eagle Medium (DMEM, Corning) supplemented with 10% fetal bovine serum (Corning) and 0.2% Primocin (InVivoGen, San Diego, Calif.) and incubated for 48 hours at 37° C., 5% CO2. On day 2, the medium was changed to DMEM (Corning) supplemented with 2% horse serum (Horse Serum, New Zealand origin, ThermoFisher, Waltham, Mass.) and 0.2% Primocin. On day 5, the medium was replaced with fresh DMEM supplemented with 2% horse serum and 0.2% Primocin.

On day 7, the DMEM supplemented with 2% horse serum and 0.2% Primocin was replaced with amino acid free DMEM (US Biologicals, Salem, Mass.) containing a defined custom amino acid concentration based on 0.5× the mean physiological concentrations in blood based on values published in the Human Metabolome Database (HMDB (Wishart D S, Tzur D, Knox C, et al., HMDB: the Human Metabolome Database. Nucleic Acids Res. 2007 January; 35(Database issue):D521-6. 17202168), with 25 mM Glucose, 1 mM Sodium Pyruvate and incubated for 2 hours at 37° C., 5% CO2. Next, the cell medium was replaced with amino acid free DMEM (US Biologicals, Salem, Mass.) containing a defined custom amino acid concentration based on 0.5× the mean physiological concentrations in blood based on values published in the HMDB and a dose curve of defined amino acid compositions listed in Table 5 at 4 doses relative to plasma levels (1×, 2×, 5× and 10×; as defined by mean amino acid concentrations in the HMDB database). Combinations containing N-acetylcysteine were dosed with 0.2 mM. Cells were treated for 30 min at 37° C., 5% CO2. After treatment, the cells were washed 1× in 100 uL cold phosphate-buffered saline 1×, pH 7.2 (PBS, ThermoFisher). For the detection of the intracellular rpS6 phosphorylation, the AlphaScreen SureFire cellular kinase assay kit (rpS6 (p-S235/236) and the AlphaScreen protein A kit (PerkinElmer, Waltham, Mass.) were used.

TABLE 5
Amino Acid compositions for mTORC1 assay.

LRQNAC
LIVRQNAC
LIVRQNACHKFT
LIV
LIVRQ
RQNAC
Q
LIVHKFTMW
NAC

Table 6 shows the results of two independent experiments assessing the ability of amino acid compositions to activate mTORC1 signaling. Data are presented as fold change of intracellular rpS6 phosphorylation in C2C12 myotubes normalized to the total protein amount compared to untreated cells. Displayed are the mean fold change in P-rpS6 for 12 biological repeats calculated from the mean of four technical replicates. Statistical significance was determined by one-way ANOVA for each composition dose relative to untreated. The combinations LRQNAC, LIVRQNAC, LIVRQNACHKFT, and LIVRHKFTWM showed significant activation of mTORC1 at all doses tested.

TABLE 6
mTORC1 activity assay dose response
to compositions described in Table 5.

| | Avg | SD | n | significance | Adj pVal |
|---|---|---|---|---|---|
| | LRQNac | | | | |
| Untreated | 1 | 0.1898 | 12 | — | — |
| 1X | 1.262 | 0.3263 | 12 | ns | 0.16 |
| 2X | 1.358 | 0.2556 | 12 | * | 0.0278 |
| 5X | 1.419 | 0.3393 | 12 | ** | 0.0072 |
| 10X | 1.472 | 0.2873 | 12 | ** | 0.0021 |
| | LIVRQNac | | | | |
| Untreated | 1 | 0.1898 | 12 | — | — |
| 1X | 1.155 | 0.1604 | 12 | *** | 0.0001 |
| 2X | 1.284 | 0.2082 | 12 | **** | <0.0001 |
| 5X | 1.264 | 0.1593 | 12 | **** | <0.0001 |
| 10X | 1.295 | 0.2115 | 12 | **** | <0.0001 |
| | LIVRQNacHKFT | | | | |
| Untreated | 1 | 0.1898 | 12 | — | — |
| 1X | 1.418 | 0.4031 | 12 | ** | 0.0073 |
| 2X | 1.35 | 0.3283 | 12 | * | 0.0317 |
| 5X | 1.491 | 0.317 | 12 | ** | 0.0012 |
| 10X | 1.518 | 0.294 | 12 | *** | 0.0006 |
| | LIV | | | | |
| Untreated | 1 | 0.07973 | 12 | — | — |
| 1X | 1.065 | 0.1027 | 12 | ns | 0.5381 |
| 2X | 1.06 | 0.1255 | 12 | ns | 0.6145 |
| 5X | 1.092 | 0.1412 | 12 | ns | 0.2154 |
| 10X | 1.145 | 0.1199 | 12 | * | 0.0169 |
| | LIVRQ | | | | |
| Untreated | 1 | 0.07973 | 12 | — | — |
| 1X | 1.04 | 0.1895 | 12 | ns | 0.9603 |
| 2X | 1.063 | 0.1176 | 12 | ns | 0.801 |
| 5X | 1.156 | 0.1561 | 12 | ns | 0.0777 |
| 10X | 1.14 | 0.1861 | 12 | ns | 0.1328 |
| | RQNac | | | | |
| Untreated | 1 | 0.07973 | 12 | — | — |
| 1X | 1.046 | 0.1279 | ns | 0.8261 | 0.001 |
| 2X | 0.8841 | 0.1355 | ns | 0.0893 | 0.0001 |
| 5X | 0.8741 | 0.1158 | ns | 0.0557 | 0.0004 |
| 10X | 0.773 | 0.1373 | *** | 0.0001 | 0.0001 |
| | LIVRHKFTMW | | | | |
| Untreated | 1 | 0.06904 | 12 | — | — |
| 1X | 1.21 | 0.1539 | 12 | ** | 0.0077 |
| 2X | 1.293 | 0.1381 | 12 | *** | 0.0001 |
| 5X | 1.327 | 0.1486 | 12 | **** | <0.0001 |
| 10X | 1.398 | 0.2227 | 12 | **** | <0.0001 |
| | Nac | | | | |
| Untreated | 0.9801 | 0.08963 | 12 | — | — |
| 1X | 0.9198 | 0.1187 | 12 | ns | 0.7719 |
| 2X | 0.8626 | 0.1092 | 12 | ns | 0.1961 |
| 5X | 0.8741 | 0.1672 | 12 | ns | 0.279 |
| 10X | 0.9485 | 0.1574 | 12 | ns | 0.9786 |
| | Q | | | | |
| Untreated | 1 | 0.06904 | 12 | — | — |
| 1X | 1.052 | 0.07853 | 12 | ns | 0.3867 |
| 2X | 0.9176 | 0.07366 | 12 | ns | 0.0616 |
| 5X | 0.8748 | 0.07727 | 12 | ** | 0.0016 |
| 10X | 0.8139 | 0.1128 | 12 | **** | <0.0001 |

### Example 2

### Promotion of Myogenesis with an Amino Acid Composition

Myogenesis is the process of forming skeletal muscle fibers (myofibers) which contain the minimal contractile units (sarcomeres) responsible for force transduction and load bearing in higher eukaryotes. During development single nucleated myogenic cells fuse, differentiate, and induce expression of the cytoskeletal complexes required for muscle contraction. A highly similar process is induced in response to muscle injury where satellite cells, or skeletal muscle stem cells, are activated, differentiate and fuse with damaged myofibers, thereby contributing myonuclei and supporting muscle repair. C2C12 cells are murine myogenic cells that, upon differentiation, fuse to form multi-nucleated myotubes (primitive myofibers) that express master regulators of muscle-specific gene expression, for example myosin heavy chain, a critical component of sarcomeres. C2C12 cells were selected as a model of myogenesis and used to test whether specific amino acid compositions would promote formation of myotubes and expression of the myotube-specific marker myosin heavy chain.

C2C12 murine myoblast cells were obtained from ATCC (CRL-1772) and seeded on day 0 at 1.0E4 cells per well in collagen I coated 96-well optical polymer microplates (ThermoFisher) in Dulbecco's Modified Eagle Medium (DMEM, Corning) supplemented with 10% heat inactivated fetal bovine serum (HI-FBS, Atlanta Biologicals) and 0.2% Primocin (InVivoGen) and incubated overnight at 37° C., 5% CO2. On day 1, cells were washed with 200 μL per well AA- and serum-free DMEM media (US Biologicals) and replaced with 1× HMDB DMEM (AA-free DMEM containing amino acids at concentrations based on the mean physiological concentrations in blood based on values published in the Human Metabolome Database (Wishart D S, Tzur D, Knox C, et al., HMDB: the Human Metabolome Database. Nucleic Acids Res. 2007 January; 35(Database issue):D521-6., which is hereby incorporated by reference in its entirety), with 6 mM Glucose, 1 mM Sodium Pyruvate, and 2% dialyzed horse serum (3.5K MWCO). Cells were treated in triplicate with defined amino acid compositions (Table 7) with increasing concentrations relative to HMDB plasma levels (1.25×, 2.5×, 5×, 10×), or control interventions 10 nM Rapamycin, 250 nM Torin1, and 100 nM insulin, combinations containing N-acetylcysteine were dosed with 1 mM. Cells were differentiated for 4 days at 37° C., 5% CO2 with a media replenishment and an additional amino acid composition or control treatment on Day 3. On day 5, the media was removed and cells were incubated in pre-warmed 4% Paraformaldehyde in PBS for 12 mins at room temperature and then washed 3× in PBS.

TABLE 7
Amino acid compositions for myogenesis assay.

LRQNAC
LIVRQNAC
LIVRQNACHKFT
LIV
LIVRQ
LIVRNAC
LIVRNACHKFT
Q
LIVRHKFTMW
LIVHKFTMW
LIVRHKFTM
LRQNACHKFT

Immunostaining with MHC (MF-20, University of Iowa Developmental Hybridoma Studies bank) was performed according to cell signaling general immunofluorescence protocol. Briefly, fixed cells were incubated in blocking buffer (5% normal goat serum 0.3% triton PBS) for 30 to 60 minutes, and then incubated overnight at 4° C. in primary antibody 1:1000 in antibody dilution buffer (1% BSA 0.3% triton in PBS). The next day the plate was equilibrated to room temperature, washed 3 times for 5 minutes in room temperature PBS and then with secondary antibody (Fab′ anti-mouse Alexa488, 1:2000) in antibody dilution buffer for 1-2 hours. Cells were washed two times for 5 minutes, incubated in Hoechst stain (Mol Probes 1:4000) for 10 minutes at room temperature, and washed an additional two times for five minutes each in PBS. Molecular device HCS was used for image acquisition and analysis. Imaging was performed with a 10× wide field objective at both GFP and UV channels (FITC and DAPI) and analysis was performed using a custom module in the MetaExpress Software measuring average FITC intensity, integrated FITC intensity, and nuclear counts.

Table 8 shows the dose responses for each composition as a fold-change to untreated and adjusted p-Values for each treatment group compared to the control. The data is the average of 3 independent experiments. The combinations LRQNAC, LIVRQNAC, LIVRQNACHKFT, and LIVRHKFTWM showed a significant increase in myotube differentiation (myogenesis) at 2.5× 5×, and 10×, with LRQNAC also showing a significant increase at 1.25×.

TABLE 8
Results of myogenesis dose response assay of amino acid compositions
described in Table 7. Summary of three myogenesis experiments,
normalized to untreated myoblast cells, are shown.

| | Ave | SD | n | sig | Adj pVal |
|---|---|---|---|---|---|
| | LRQNac | | | | |
| Untreated | 1.000 | 0.122 | 3 | — | — |
| 1.25X | 1.264 | 0.283 | 3 | ** | 0.0029 |
| 2.5X | 1.449 | 0.174 | 3 | **** | 0.0001 |
| 5X | 1.472 | 0.225 | 3 | **** | 0.0001 |
| 10X | 1.275 | 0.152 | 3 | ** | 0.0018 |
| | LIVRQNac | | | | |
| Untreated | 1.000 | 0.122 | 3 | — | — |
| 1.25X | 1.136 | 0.243 | 3 | ns | 0.1539 |
| 2.5X | 1.281 | 0.193 | 3 | *** | 0.0003 |
| 5X | 1.275 | 0.095 | 3 | *** | 0.0004 |
| 10X | 1.226 | 0.191 | 3 | ** | 0.0045 |
| | LIVRQNacHKFT | | | | |
| Untreated | 1.000 | 0.122 | 3 | — | — |
| 1.25X | 1.185 | 0.132 | 3 | ** | 0.0073 |
| 2.5X | 1.305 | 0.070 | 3 | **** | 0.0001 |
| 5X | 1.344 | 0.097 | 3 | **** | 0.0001 |
| 10X | 1.187 | 0.140 | 3 | ** | 0.0069 |
| | LRQNacHKFT | | | | |
| Untreated | 1.000 | 0.134 | 3 | — | — |
| 1.25X | 1.110 | 0.219 | 3 | ns | 0.6122 |
| 2.5X | 1.578 | 0.299 | 3 | **** | 0.0001 |
| 5X | 1.558 | 0.205 | 3 | **** | 0.0001 |
| 10X | 1.478 | 0.193 | 3 | **** | 0.0001 |
| | LIVRQ | | | | |
| Untreated | 1.000 | 0.149 | 3 | — | — |
| 1.25X | 1.422 | 0.255 | 3 | **** | 0.0001 |
| 2.5X | 1.382 | 0.230 | 3 | **** | 0.0001 |
| 5X | 1.376 | 0.178 | 3 | **** | 0.0001 |
| 10X | 1.223 | 0.166 | 3 | * | 0.029 |
| | LIVRNac | | | | |
| Untreated | 1.000 | 0.149 | 3 | — | — |
| 1.25X | 1.422 | 0.255 | 3 | ns | 0.1409 |
| 2.5X | 1.382 | 0.230 | 3 | **** | 0.0001 |
| 5X | 1.376 | 0.178 | 3 | **** | 0.0001 |
| 10X | 1.223 | 0.166 | 3 | **** | 0.0001 |
| | LIVRNacHKFT | | | | |
| Untreated | 1.000 | 0.140 | 3 | — | — |
| 1.25X | 1.054 | 0.160 | 3 | ns | 0.9457 |
| 2.5X | 1.329 | 0.181 | 3 | *** | 0.0008 |
| 5X | 1.406 | 0.278 | 3 | **** | 0.0001 |
| 10X | 1.260 | 0.157 | 3 | * | 0.0104 |
| | Q | | | | |
| Untreated | 1.000 | 0.146 | 3 | — | — |
| 1.25X | 1.127 | 0.066 | 3 | ns | 0.1196 |
| 2.5X | 1.066 | 0.130 | 3 | ns | 0.6767 |
| 5X | 1.087 | 0.214 | 3 | ns | 0.4175 |
| 10X | 1.041 | 0.111 | 3 | ns | 0.9299 |
| | LIVRHKFTMW | | | | |
| Untreated | 1.000 | 0.116 | 3 | — | — |
| 1.25X | 0.843 | 0.136 | 3 | ns | 0.1282 |
| 2.5X | 1.055 | 0.241 | 3 | ns | 0.906 |
| 5X | 0.991 | 0.145 | 3 | ns | 0.9998 |
| 10X | 1.038 | 0.193 | 3 | ns | 0.9797 |
| | LIVHKFTMW | | | | |
| Untreated | 1.000 | 0.109 | 3 | — | — |
| 1.25X | 0.873 | 0.128 | 3 | ns | 0.2377 |
| 2.5X | 1.036 | 0.107 | 3 | ns | 0.9777 |
| 5X | 1.006 | 0.169 | 3 | ns | 0.9999 |
| 10X | 1.053 | 0.231 | 3 | ns | 0.8987 |
| | LIVRHKFTM | | | | |
| Untreated | 1.000 | 0.109 | 3 | — | — |
| 1.25X | 1.180 | 0.166 | 3 | ns | 0.0776 |
| 2.5X | 1.174 | 0.274 | 3 | ns | 0.0941 |
| 5X | 1.127 | 0.193 | 3 | ns | 0.3202 |
| 10X | 1.077 | 0.145 | 3 | ns | 0.7543 |
| | LIV | | | | |
| Untreated | 1.000 | 0.149 | 3 | — | — |
| 1.25X | 1.259 | 0.256 | 3 | * | 0.0165 |
| 2.5X | 1.223 | 0.165 | 3 | * | 0.0485 |
| 5X | 1.252 | 0.188 | 3 | * | 0.0205 |
| 10X | 1.222 | 0.298 | 3 | ns | 0.0503 |

### Example 3

### Promotion of Myotube Growth with an Amino Acid Composition

Myotubes are multi-nucleated and elongated cells that express master regulators of skeletal muscle gene expression including MyoD and Myogenin. Myotubes are formed by differentiating myoblasts (muscle progenitor cells) for approximately 1 week. Once formed, myotubes size can be promoted (e.g. insulin) or inhibited (e.g. myostatin) with various molecules in order to assess effects on muscle size in vitro. C2C12 cells are commonly-used murine myogenic cells that upon differentiation form myotubes. C2C12 myotubes were used to test whether specific amino acid compositions can promote growth in vitro.

C2C12 murine myoblast cells (ATCC CRL-1772) were seeded on day 0 at 1.0E4 cells per well in collagen I coated 96-well optical polymer microplates (ThermoFisher) in Dulbecco's Modified Eagle Medium (DMEM, Corning) supplemented with 10% heat inactivated fetal bovine serum (HI-FBS, Atlanta Biologicals) and 0.2% Primocin (InVivoGen) and incubated overnight at 37° C., 5% CO2. On day 1, media was washed and differentiation media (DMEM supplemented with 2% horse serum) was added to the cells and fresh differentiation media was also applied on Day 3. On Day 6, cells were washed with amino acid-free DMEM and then treated with basal growth media containing 0.2% dialyzed FBS and all amino acids at 0.25× concentrations found in plasma based on values reported in the Human Metabolome Database (HMDB). Additionally, cells were treated in triplicate with the amino acid compositions listed in Table 9 at 4 doses relative to plasma levels (1.25×, 2.5×, 5×, 10×), or control interventions 10 nM Rapamycin, 250 nM Torin1, and 100 nM insulin, combinations containing N-acetylcysteine were dosed with 1 mM.

TABLE 9
Amino acid compositions for myotube growth assay.

LRQNAC
LIVRQNAC
LIVRQNACHKFT
LIV
LIVRQ
Q
LIVRHKFTM
LIVHKFTMW
LIVRHKFTMW
LRQNACHKFT
RQNAC
NAC

Fresh growth media and AA treatments were applied a second time on Day 8. On day 10, media was removed and cells were incubated in pre-warmed 4% Paraformaldehyde in PBS for 12 mins at room temperature and then washed 3× in PBS. Immunostaining with MHC (MF-20, University of Iowa Developmental Hybridoma Studies bank) was performed according to cell signaling general immunofluorescence protocol. Briefly, fixed cells were incubated in blocking buffer (5% normal goat serum 0.3% triton PBS) for 30-60 minutes, and then incubated overnight at 4° C. in primary antibody 1:1000 in antibody dilution buffer (1% BSA 0.3% triton in PBS). The next day the plate was equilibrated to room temperature, washed 3 times for 5 minutes in room temperature PBS and then with secondary antibody (Fab′ anti-mouse Alexa488, 1:2000) in antibody dilution buffer for 1-2 hours. Cells were washed 2 times for 5 minutes, incubated in Hoechst stain (Mol Probes 1:4000) for 10 minutes at room temperature, and washed an additional 2 times five minutes each in PBS. Molecular device HCS was used for image acquisition and analysis. Imaging was performed with a 10× wide field objective at both GFP and UV channels (FITC and DAPI) and analysis was performed using a modified version of the angiogenesis module in the MetaExpress Software measuring average total myotube area, myotube breadth, total nuclear counts, fused nuclear counts, and unfused nuclear counts.

Table 10 summarizes data across two experiments for 2.5× treatment group for the area of the well covered by myotubes (image data for myotube area is normalized to nuclear count for each well, the table displays average of six images per well and approximately 6 wells per experiment, 12 wells total). Consistently, the LRQNAC, LIVRQNAC, LIVRQNacHKFT, LIVHKFTMW, LRQNacHKFT, and RQNAC significantly increased the area of myotubes in the culture well while other compositions, such as LIV or Q, had no effect or were inhibitory.

TABLE 10
Results of myotube growth dose response assay of
amino acid compositions described in Table 9.

| | Avg | SD | n | sig | Adj pVal |
|---|---|---|---|---|---|
| | LRQNac | | | | |
| Untreated | 1.000 | 0.163 | 12 | — | — |
| 1.25X | 1.342 | 0.083 | 5 | *** | 0.0004 |
| 2.5X | 1.314 | 0.147 | 5 | ** | 0.001 |
| 5X | 1.626 | 0.175 | 4 | **** | <0.0001 |
| 10X | 1.498 | 0.066 | 5 | **** | <0.0001 |
| | LIVRQNac | | | | |
| Untreated | 1.000 | 0.163 | 12 | — | — |
| 1.25X | 1.324 | 0.087 | 6 | *** | 0.0001 |
| 2.5X | 1.444 | 0.138 | 6 | **** | <0.0001 |
| 5X | 1.561 | 0.141 | 6 | **** | <0.0001 |
| 10X | 1.527 | 0.088 | 6 | **** | <0.0001 |
| | LIVRQNacHKFT | | | | |
| Untreated | 1.000 | 0.163 | 12 | — | — |
| 1.25X | 1.335 | 0.273 | 6 | ** | 0.0024 |
| 2.5X | 1.379 | 0.201 | 6 | *** | 0.0007 |
| 5X | 1.633 | 0.096 | 6 | **** | <0.0001 |
| 10X | 1.533 | 0.109 | 6 | **** | <0.0001 |
| | LRQNacHKFT | | | | |
| Untreated | 1.000 | 0.163 | 12 | — | — |
| 1.25X | 1.377 | 0.276 | 5 | ** | 0.0014 |
| 2.5X | 1.471 | 0.137 | 6 | **** | <0.0001 |
| 5X | 1.355 | 0.152 | 4 | ** | 0.0054 |
| 10X | 1.405 | 0.121 | 5 | *** | 0.0006 |
| | LIVRQ | | | | |
| Untreated | 1.000 | 0.163 | 12 | — | — |
| 1.25X | 1.210 | 0.235 | 4 | ns | 0.1307 |
| 2.5X | 1.186 | 0.069 | 4 | ns | 0.2059 |
| 5X | 1.127 | 0.191 | 4 | ns | 0.5192 |
| 10X | 1.112 | 0.169 | 6 | ns | 0.5016 |
| | LIV | | | | |
| Untreated | 0.659 | 0.145 | 6 | — | — |
| 1.25X | 0.963 | 0.109 | 6 | ns | 0.9561 |
| 2.5X | 0.882 | 0.137 | 6 | ns | 0.2881 |
| 5X | 0.894 | 0.099 | 5 | ns | 0.4302 |
| 10X | 0.659 | 0.145 | 6 | *** | 0.0001 |
| | LIVRHKFTM | | | | |
| Untreated | 1.000 | 0.163 | 12 | — | — |
| 1.25X | 1.024 | 0.134 | 5 | ns | 0.9871 |
| 2.5X | 1.003 | 0.030 | 6 | ns | >0.9999 |
| 5X | 1.035 | 0.065 | 6 | ns | 0.9342 |
| 10X | 1.067 | 0.062 | 5 | ns | 0.671 |
| | LIVHKFTMW | | | | |
| Untreated | 1.000 | 0.163 | 12 | — | — |
| 1.25X | 1.149 | 0.075 | 5 | ns | 0.6107 |
| 2.5X | 0.625 | 0.492 | 6 | * | 0.0162 |
| 5X | 0.741 | 0.090 | 4 | ns | 0.2217 |
| 10X | 0.765 | 0.106 | 5 | ns | 0.2326 |
| | LIVRHKFTMW | | | | |
| Untreated | 1.000 | 0.163 | 12 | — | — |
| 1.25X | 1.102 | 0.158 | 6 | ns | 0.5181 |
| 2.5X | 0.997 | 0.092 | 6 | ns | >0.9999 |
| 5X | 0.860 | 0.226 | 5 | ns | 0.3045 |
| 10X | 0.813 | 0.137 | 6 | ns | 0.0811 |
| | Q | | | | |
| Untreated | 1.000 | 0.163 | 12 | — | — |
| 1.25X | 1.235 | 0.176 | 6 | * | 0.0375 |
| 2.5X | 1.202 | 0.254 | 5 | ns | 0.1129 |
| 5X | 1.075 | 0.070 | 4 | ns | 0.8644 |
| 10X | 1.077 | 0.152 | 6 | ns | 0.7878 |
| | RQNac | | | | |
| Untreated | 1.000 | 0.163 | 12 | — | — |
| 1.25X | 1.270 | 0.128 | 6 | ** | 0.001 |
| 2.5X | 1.324 | 0.117 | 6 | *** | 0.0001 |
| 5X | 1.293 | 0.084 | 6 | *** | 0.0004 |
| 10X | 1.317 | 0.107 | 6 | *** | 0.0001 |
| | Nac | | | | |
| Untreated | 1.000 | 0.163 | 12 | — | — |
| 1.25X | 1.121 | 0.085 | 6 | ns | 0.223 |
| 2.5X | 1.036 | 0.074 | 6 | ns | 0.9525 |
| 5X | 1.153 | 0.158 | 5 | ns | 0.1171 |
| 10X | 1.267 | 0.119 | 6 | ** | 0.0013 |

### Summary of Examples 1-3

As summarized in Table 11, across all assays and experiments, only the amino acid compositions of the present disclosure were able to significantly induce activity compared to the untreated control, for doses between 2× and 5×.

TABLE 11
Summary of statistically significant assay
results between 2X and 5X doses.

| | MYOGENESIS | mTOR | GROWTH |
|---|---|---|---|
| LRQNac | X | X | X |
| LIVRQNac | x | X | X |
| LIVRQNacHKFT | X | X | X |
| LRQNacHKFT | X | NT | X |
| LIV | X | ns | ns |
| LIVRQ | X | ns | ns |
| Q | ns | ns | ns |
| LIVRHKFTM | ns | NT | ns |
| LIVHKFTMW | ns | NT | ns |
| LIVRHKFTMW | ns | X | ns |
| RQNac | NT | ns | X |
| Nac | NT | ns | ns |

ns—not significant
NT—not tested

Muscle diseases are complex and driven by a multitude of unique mechanisms. Recovery from muscle loss or injury requires coordination of many biological, cellular, and molecular processes. The amino acid compositions defined herein are designed to promote muscle growth and function for a wide range of muscle pathologies. The amino acid compositions disclosed in this application are able to promote mTORC1-dependent cellular anabolism, muscle cell differentiation, and muscle growth, whereas compositions, such as LIV and Q, are only able to influence some, but not all of those important processes required for maintaining muscle health.

### Example 4

### Treatment of Immobilization in Subjects with an Amino Acid Composition

The study described herein features the administration of a composition including amino acids to healthy subjects undergoing unilateral knee immobilization. The goal of this study was to determine the impact of an amino acid composition on muscle atrophy after 7 days of single leg immobilization and 14 days of recovery post-immobilization. The composition included about 1 g of L-leucine, about 0.5 g of L-isoleucine, about 0.5 g of L-valine, about 1.5 g of L-arginine (or 1.81 g of L-arginine HCl), about 1.33 g of L-glutamine, about 0.15 g of N-acetylcysteine, about 0.08 g of L-histidine, about 0.35 g of L-lysine, about 0.08 g of L-phenylalanine, and about 0.17 g of L-threonine per stick packet for administration in four stick packs three times per day (e.g., a total of about 68 or 72 g per day, or about 23 g or 24 g three times per day).

In a clinical study, subjects received the amino acid composition three times daily for 28 days. Amino acids were provided in powder form to be dissolved in 8 oz. of water. Participants underwent single-leg immobilization for 7 days (days 8-15) during the 28 day study period. An immobilization device was used for 7 days of single-leg immobilization of the dominant knee (based on maximal isometric leg strength) with a knee brace worn in a fixed flexion position at 140° (e.g., a Breg brace).

Control subjects received placebo three times daily for 28 days. Placebo consisted of an amount of maltodextrin (NF grade) equivalent to the amount of amino acids administered, dissolved in 8 oz. of water. Participants underwent single-leg immobilization for 7 days (days 8-15) during the 28 day study period.

The primary outcome measure of this study was safety and tolerability. In addition, muscle disuse atrophy, in particular, the impact of the amino acid formulation on muscle atrophy after 7 days of single leg immobilization was studied. The secondary outcome measures included muscle function based on knee strength, muscle cross-section area and volume, muscle fiber quality, and lean muscle mass. The percentage change in lean muscle mass in the subjects was determined using dual-energy x-ray absorptiometry (DEXA). The percentage change in maximum torque as measured using a BioDex machine (measured in Newton-meters) and percentage change in the time to maximum torque (measured in seconds) were also assessed. Muscle biopsies will be performed to determine muscle fiber cross-sectional area (CSA). Muscle size will also be assessed via MRI. Muscle health will be assessed by electrical impedance myography (EIM) measurements. Assessments were performed at baseline (day 1), pre-immobilization (day 8), post-immobilization (day 15), and recovery (day 28).

Key criteria for selecting subjects included the following: 1) generally healthy, non-smoking; 2) willing and able to provide informed consent; 3) men age 20-45 years; and 4) BMI between 25 and 35 kg/m². Exclusion Criteria included the following: 1) smokers; 2) subject has any concurrent medical, orthopedic, or psychiatric condition that, in the opinion of the investigator, would compromise his/her ability to comply with the study requirements; 3) history of cancer within the last 5 years, except basal cell carcinoma, non-squamous skin carcinoma, prostate cancer, or carcinoma in situ with no significant progression over the past 2 years; 4) significant orthopedic, cardiovascular, pulmonary, renal, liver, infectious disease, immune disorder (requiring ongoing medical care), or metabolic/endocrine disorder (e.g., diabetes, high cholesterol, elevated fasting blood sugar) or other disease that would preclude oral protein supplement ingestion and/or assessment of safety and study objectives; 5) any cachexia-related condition (e.g., relating to cancer, tuberculosis, or human immunodeficiency virus infection and acquired immune deficiency syndrome) or any genetic muscle diseases or disorders; 6) current illnesses that could interfere with the study (e.g. prolonged severe diarrhea, regurgitation, or difficulty swallowing); 7) subject participated in a study of an investigational product less than 60 days or 5 half-lives of the investigational product, whichever is longer, before enrollment in this study; 8) hypersensitivity to any of the components of the test product; 9) excessive alcohol consumption (>21 units/week); 10) known sensitivity or allergy to amino acids or any ingredient in the test formulations; 11) prior gastrointestinal bypass surgery (e.g., lapband surgery), irritable bowel disease, or irritable bowel syndrome; 12) history of bleeding diathesis, platelet or coagulation disorders, or antiplatelet/anticoagulation therapy (up to 81 mg of baby aspirin per day taken as a prophylactic is permitted); 13) personal or family history of clotting disorder or deep vein thrombosis; 14) concomitant use of corticosteroids, testosterone replacement therapy (ingestion, injection, or transdermal), any anabolic steroid, creatine, whey protein supplements, casein, or branched-chain amino acids (BCAAs) within 45 days prior to screening; 15) contraindications to an MRI scan (e.g. subjects with non-removable ferromagnetic implants, pacemakers, aneurysm clips or other foreign bodies, or subjects with claustrophobic symptoms that would contraindicate an MRI scan); 16) hemoglobin less than 11.5 mg/dl at screening; or 17) platelets less than 150,000/uL (150×109/L) at screening.

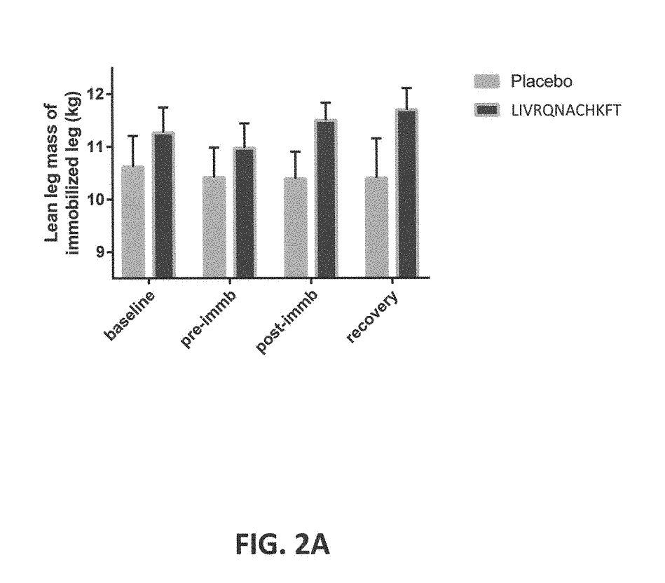

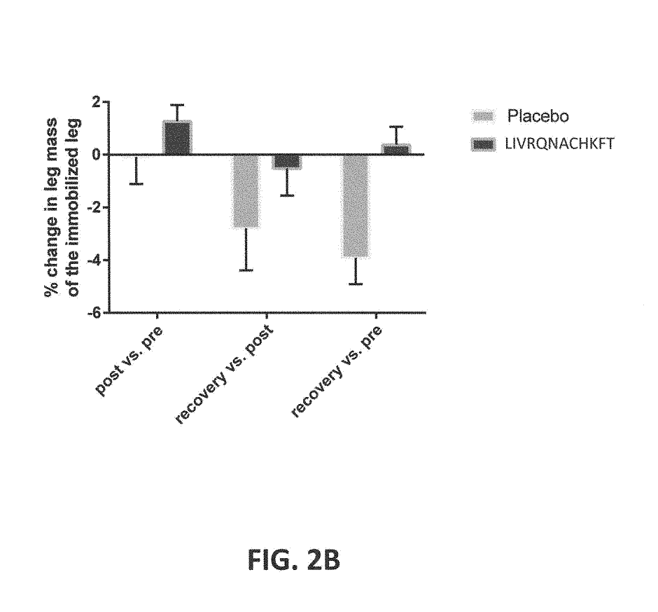

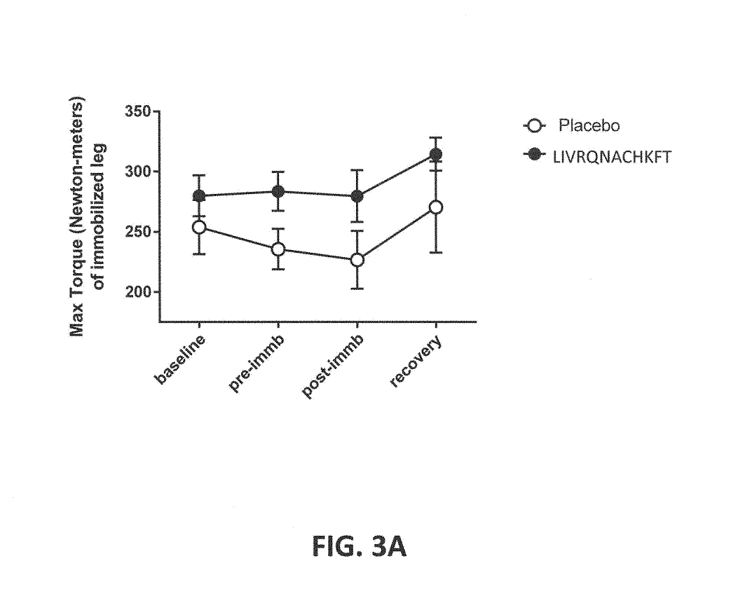

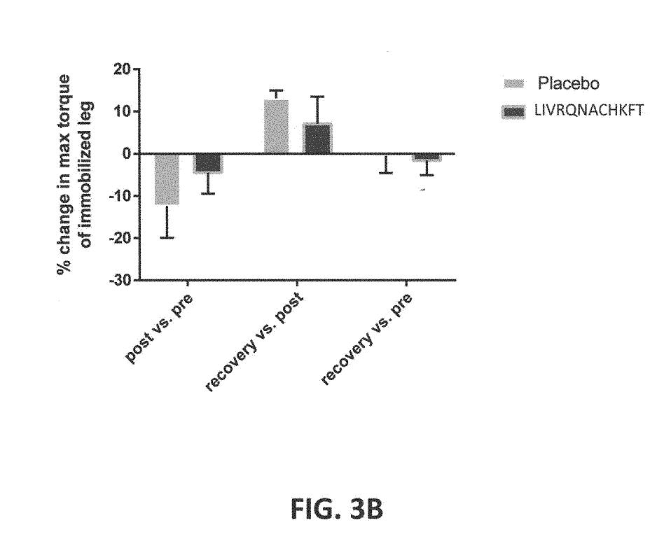

The findings from this study suggest that the decline in lean leg mass as a result of unilateral limb immobilization (i.e. disuse atrophy) was attenuated in those that received the LIVRQNACHKFT amino acid combination, as compared to those that received placebo. These results in subjects undergoing a unilateral limb immobilization suggest that the amino acid combination attenuated this decline in lean mass of the immobilized leg (FIGS. 2A and 2B; Tables 12 and 13), while preserving muscle strength (FIGS. 3A and 3B; Tables 16 and 17). The immobilized leg in the placebo administered groups did not recover their lean mass to the post-immobilized or the pre-immobilized state during the two week recovery period. By contrast, administration of the amino acid combination maintained and/or improved the lean leg mass within this two week recovery period to that of the post and pre-immobilization levels (see, FIG. 2B, recovery vs. post-immob and recovery vs pre-immob columns). The decline in muscle strength seen after a week of unilateral limb immobilization in the placebo group was also attenuated by the amino acid combination (see FIG. 3B, post vs. pre column). The non-immobilized leg in either the Placebo or the LIVRQNACHKFT amino acid administered group did not appear to lose their lean leg mass nor their muscle strength to the same extent as the corresponding immobilized leg during the knee brace period, as expected of an appropriate control.

TABLE 12
Lean leg mass (kg) of the immobilized leg by DXA.

| | Placebo | | | LIVRQNACHKFT | | |
|---|---|---|---|---|---|---|
| | Mean | SEM | N | Mean | SEM | N |
| baseline (Day 1) | 10.62 | 0.59 | 10 | 11.27 | 0.48 | 10 |
| pre-immb (Day 8) | 10.42 | 0.56 | 10 | 10.97 | 0.47 | 10 |
| post-immb (Day 15 | 10.39 | 0.51 | 10 | 11.5 | 0.33 | 9 |
| recovery (Day 28) | 10.4 | 0.75 | 7 | 11.7 | 0.41 | 6 |

TABLE 13
% change in lean leg mass of the immobilized leg at key points.

| | Placebo | | | LIVRQNACHKFT | | |
|---|---|---|---|---|---|---|
| | Mean | SEM | N | Mean | SEM | N |
| post-immb vs. pre-immmb | −0.09 | 1.02 | 10 | 1.26 | 0.62 | 9 |
| recovery vs. post-immb | −2.79 | 1.6 | 7 | −0.53 | 1.02 | 6 |
| recovery vs. pre-immb | −3.92 | 0.99 | 7 | 0.37 | 0.68 | 6 |

The non-immobilized leg in either the Placebo or LIVRQNACHKFT groups does not appear to lose lean mass to the same extent as the corresponding immobilized leg during the knee brace period, as expected of an appropriate control. Further, LIVRQNACHKFT administration appears to improve recovery after immobilization (i.e. immobilized leg) more so, as compared to the non-immobilized leg (Tables 14 and 15):

TABLE 14
% change in lean leg mass in the NON-immobilized leg: Placebo.

| | Day 15 vs. Day 8 | Day 28 vs. Day 15 | Day 28 vs. Day 8 |
|---|---|---|---|
| Mean | 0.44 | −1.85 | −1.69 |
| SEM | 0.86 | 1.12 | 0.87 |

TABLE 15
% change in lean leg mass in the NON-
immobilized leg: LIVRQNACHKFT.

| | | | |
|---|---|---|---|
| Mean | 1.13 | −1.19 | −0.01 |
| SEM | 1.17 | 0.33 | 0.78 |

TABLE 16
Max Torque (Newton-meters) of the immobilized
leg by strength assessment.

| | Placebo | | | LIVRQNACHKFT | | |
|---|---|---|---|---|---|---|
| | Mean | SEM | N | Mean | SEM | N |
| baseline (Day 1) | 253.9 | 22.59 | 10 | 279.9 | 16.97 | 9 |
| pre-immb (Day 8) | 235.6 | 16.97 | 10 | 283.6 | 16.02 | 9 |
| post-immb (Day 15) | 226.7 | 24.1 | 7 | 279.6 | 21.45 | 7 |
| recovery (Day 28) | 270.5 | 37.82 | 3 | 314.4 | 13.83 | 5 |

TABLE 17
% change in max torque of the immobilized leg at key points.

| | Placebo | | | LIVRQNACHKFT | | |
|---|---|---|---|---|---|---|
| | Mean | SEM | N | Mean | SEM | N |
| post-immb vs. pre-immb | −12.4 | 7.55 | 7 | −4.5 | 4.99 | 7 |
| recovery vs. post-immb | 13.1 | 1.85 | 3 | 7.1 | 6.33 | 5 |
| recovery vs. pre-immb | −0.5 | 4.12 | 3 | −1.6 | 3.5 | 5 |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A composition comprising:
a) a leucine (L)-amino acid entity chosen from Table 2 or a salt thereof, a dipeptide or salt thereof, a tripeptide or salt thereof, or a combination of any of the aforesaid;
b) an arginine (R)-amino acid entity chosen from Table 2 or a salt thereof, a dipeptide or salt thereof, a tripeptide or salt thereof, or a combination of any of the aforesaid;
c) a glutamine (Q)-amino acid entity chosen from Table 2 or a salt thereof, a dipeptide or salt thereof, a tripeptide or salt thereof, or a combination of any of the aforesaid;
d) N-acetylcysteine (NAC) or a salt thereof, a dipeptide or salt thereof, a tripeptide or salt thereof, or a combination of any of the aforesaid; and
e) an essential amino acid (EAA)-entity chosen from a histidine (H)-amino acid-entity, a lysine (K)-amino acid-entity, a phenylalanine (F)-amino acid-entity, and a threonine (T)-amino acid-entity or a combination of two, three, or four of the EAAs;
provided that:
the R-amino acid entity is present at a higher amount (wt. %) than the L-amino acid entity.

2. (canceled)

3. The composition of claim 1, wherein one, two, three, or more of methionine (M), trytophan (W), valine (V), or cysteine (C) is absent, or if present, is present at less than 10 weight (wt.) %, 9 wt. %, 8 wt. %, 7 wt. %, 6 wt. %, 5 wt. %, 4 wt. %, 3 wt. %, 2 wt. %, or 1 wt. %.

4. The composition of claim 1, wherein the total wt. % of (a)-(e) is greater than the total wt. % of any other amino acid entity in the composition.

5. The composition of claim 1, wherein:
f) a wt. % of the R-amino acid entity in the composition is greater than the wt. % of the Q-amino acid entity;
g) the wt. % of the Q-amino acid entity in the composition is greater than the wt. % of the L-amino acid entity;
h) the wt. % of the R-amino acid entity in the composition is greater than the wt. % of the L-amino acid entity;
i) the wt. % of the R-amino acid entity in the composition is greater than the wt. % of the EAA, or the combination of two, three, or four of the EAAs;
j) the wt. % of the Q-amino acid entity in the composition is greater than the wt. % of the EAA or the combination of two, three, or four of the EAAs;
k) the wt. % of the L-amino acid entity in the composition is greater than the wt. % of the EAA or the combination of two, three, or four of the EAAs; or
l) a combination of two, three, four, five, or six of (f)-(k).

6.-12. (canceled)

13. The composition of claim 1, further comprising one or both of an isoleucine (I)-amino acid-entity and a valine (V)-amino acid-entity.

14.-38. (canceled)

39. A pharmaceutical composition comprising the composition of claim 1 and a pharmaceutically acceptable excipient.

40. (canceled)

41. A method for improving muscle function, wherein the method comprises administering to a subject in need thereof an effective amount of a composition comprising:
a) a leucine (L)-amino acid entity chosen from Table 2 or a salt thereof, a dipeptide or salt thereof, a tripeptide or salt thereof, or a combination of any of the aforesaid;
b) an arginine (R)-amino acid entity chosen from Table 2 or a salt thereof, a dipeptide or salt thereof, a tripeptide or salt thereof, or a combination of any of the aforesaid;
c) a glutamine (Q)-amino acid entity chosen from Table 2 or a salt thereof, a dipeptide or salt thereof, a tripeptide or salt thereof, or a combination of any of the aforesaid;
d) N-acetylcysteine (NAC) or a salt thereof, a dipeptide or salt thereof, a tripeptide or salt thereof, or a combination of any of the aforesaid; and
e) an essential amino acid (EAA)-entity chosen from a histidine (H)-amino acid-entity, a lysine (K)-amino acid-entity, a phenylalanine (F)-amino acid-entity, and a threonine (T)-amino acid-entity or a combination of two, three, or four of the EAAs;
provided that:
the R-amino acid entity is present at a higher amount (wt. %) than the L-amino acid entity.

42. The method of claim 41, wherein the subject has a disease or disorder chosen from a rare muscle disease, muscle atrophy, sarcopenia, muscle deterioration, muscle decay, cachexia, drug-induced myopathy, muscular dystrophy, myopenia, muscle weakness, perceived muscle weakness, ICU-acquired myopathy, burns-related myopathy, a neuromuscular disorder, ventilator-induced diaphragmatic dystrophy, hyponatremia, hypokalemia, a calcium deficiency, hypercalcemia, amyotrophic lateral sclerosis, and a bone weakness disease, or a combination thereof.

43. The method of claim 41, wherein the subject has or is identified as having decreased muscle function due to aging, injury, muscle atrophy, infection, disease, stroke, or a fracture or other trauma.

44. The method of claim 41, wherein the subject has had a rotator cuff surgery, knee surgery, hip surgery, joint replacement, injury repair surgery, or has worn a cast prior to administration of the composition.

45.-46. (canceled)

47. A method of providing amino acid entities to a subject comprising administering to the subject an effective amount of a composition comprising:
a) a leucine (L)-amino acid entity chosen from Table 2 or a salt thereof, a dipeptide or salt thereof, a tripeptide or salt thereof, or a combination of any of the aforesaid;
b) an arginine (R)-amino acid entity chosen from Table 2 or a salt thereof, a dipeptide or salt thereof, a tripeptide or salt thereof, or a combination of any of the aforesaid;
c) a glutamine (Q)-amino acid entity chosen from Table 2 or a salt thereof, a dipeptide or salt thereof, a tripeptide or salt thereof, or a combination of any of the aforesaid;
d) N-acetylcysteine (NAC) or a salt thereof, a dipeptide or salt thereof, a tripeptide or salt thereof, or a combination of any of the aforesaid; and
e) an essential amino acid (EAA)-entity chosen from a histidine (H)-amino acid-entity, a lysine (K)-amino acid-entity, a phenylalanine (F)-amino acid-entity, and a threonine (T)-amino acid-entity or a combination of two, three, or four of the EAAs;
provided that:
the R-amino acid entity is present at a higher amount (wt. %) than the L-amino acid entity.

48. A method of manufacturing or making a composition comprising forming a composition comprising the following:
a) a L-amino acid entity chosen from Table 2 or a salt thereof, a dipeptide or salt thereof, a tripeptide or salt thereof, or a combination of any of the aforesaid;
b) an R-amino acid entity chosen from Table 2 or a salt thereof, a dipeptide or salt thereof, a tripeptide or salt thereof, or a combination of any of the aforesaid;
c) a Q-amino acid entity chosen from Table 2 or a salt thereof, a dipeptide or salt thereof, a tripeptide or salt thereof, or a combination of any of the aforesaid;
d) NAC or a salt thereof, a dipeptide or salt thereof, a tripeptide or salt thereof, or a combination of any of the aforesaid; and
e) an EAA-entity chosen from a H-amino acid-entity, a K-amino acid-entity, a F-amino acid-entity, and a T-amino acid-entity or a combination of two, three, or four of the EAAs; provided that:
the R-amino acid entity is present at a higher amount (wt. %) than the L-amino acid entity.

49.-50. (canceled)

51. The method of claim 41, wherein one, two, three, or more of M, W, V, or C is absent, or if present, is present at less than 10 weight (wt.) %, 9 wt. %, 8 wt. %, 7 wt. %, 6 wt. %, 5 wt. %, 4 wt. %, 3 wt. %, 2 wt. %, or 1 wt. %.

52. The method of claim 41, wherein the total wt. % of (a)-(e) is greater than the total wt. % of any other amino acid entity in the composition.

53. The method of claim 41, wherein:
f) a wt. % of the R-amino acid entity in the composition is greater than the wt. % of the Q-amino acid entity;
g) the wt. % of the Q-amino acid entity in the composition is greater than the wt. % of the L-amino acid entity;
h) the wt. % of the R-amino acid entity in the composition is greater than the wt. % of the L-amino acid entity;
i) the wt. % of the R-amino acid entity in the composition is greater than the wt. % of the EAA, or the combination of two, three, or four of the EAAs;
j) the wt. % of the Q-amino acid entity in the composition is greater than the wt. % of the EAA or the combination of two, three, or four of the EAAs;
k) the wt. % of the L-amino acid entity in the composition is greater than the wt. % of the EAA or the combination of two, three, or four of the EAAs; or
l) a combination of two, three, four, five, or six of (f)-(k).

54. The method of claim 41, wherein the composition further comprises one or both of an I-amino acid-entity and a V-amino acid-entity.

55. The method of claim 41, wherein the composition is formulated as a pharmaceutical composition comprising a pharmaceutically acceptable excipient.

56. The method of claim 47, wherein one, two, three, or more of methionine M, W, V, or C is absent, or if present, is present at less than 10 weight (wt.) %, 9 wt. %, 8 wt. %, 7 wt. %, 6 wt. %, 5 wt. %, 4 wt. %, 3 wt. %, 2 wt. %, or 1 wt. %.

57. The method of claim 47, wherein the total wt. % of (a)-(e) is greater than the total wt. % of any other amino acid entity in the composition.

58. The method of claim 47, wherein:
f) a wt. % of the R-amino acid entity in the composition is greater than the wt. % of the Q-amino acid entity;
g) the wt. % of the Q-amino acid entity in the composition is greater than the wt. % of the L-amino acid entity;
h) the wt. % of the R-amino acid entity in the composition is greater than the wt. % of the L-amino acid entity;
i) the wt. % of the R-amino acid entity in the composition is greater than the wt. % of the EAA, or the combination of two, three, or four of the EAAs;
j) the wt. % of the Q-amino acid entity in the composition is greater than the wt. % of the EAA or the combination of two, three, or four of the EAAs;
k) the wt. % of the L-amino acid entity in the composition is greater than the wt. % of the EAA or the combination of two, three, or four of the EAAs; or
l) a combination of two, three, four, five, or six of (f)-(k).

59. The method of claim 47, wherein the composition further comprises one or both of an I-amino acid-entity and a V-amino acid-entity.

60. The method of claim 47, wherein the composition is formulated as a pharmaceutical composition comprising a pharmaceutically acceptable excipient.

61. The method of claim 48, wherein one, two, three, or more of M, W, V, or C is absent, or if present, is present at less than 10 weight (wt.) %, 9 wt. %, 8 wt. %, 7 wt. %, 6 wt. %, 5 wt. %, 4 wt. %, 3 wt. %, 2 wt. %, or 1 wt. %.

62. The method of claim 48, wherein the total wt. % of (a)-(e) is greater than the total wt. % of any other amino acid entity in the composition.

63. The method of claim 48, wherein:
f) a wt. % of the R-amino acid entity in the composition is greater than the wt. % of the Q-amino acid entity;
g) the wt. % of the Q-amino acid entity in the composition is greater than the wt. % of the L-amino acid entity;
h) the wt. % of the R-amino acid entity in the composition is greater than the wt. % of the L-amino acid entity;
i) the wt. % of the R-amino acid entity in the composition is greater than the wt. % of the EAA, or the combination of two, three, or four of the EAAs;
j) the wt. % of the Q-amino acid entity in the composition is greater than the wt. % of the EAA or the combination of two, three, or four of the EAAs;
k) the wt. % of the L-amino acid entity in the composition is greater than the wt. % of the EAA or the combination of two, three, or four of the EAAs; or
l) a combination of two, three, four, five, or six of (f)-(k).

64. The method of claim 48, wherein the composition further comprises one or both of an I-amino acid-entity and a V-amino acid-entity.

65. The method of claim 48, wherein the composition is formulated as a pharmaceutical composition comprising a pharmaceutically acceptable excipient.
